(19) United States
(12) Patent Application Publication
(10) Pub. No.: US 2024/0294959 A1
(43) Pub. Date: Sep. 5, 2024

(54) CELLULAR PRODUCTION OF BIOPRODUCTS

(71) Applicant: INBIOSE N.V., Zwijnaarde (BE)

(72) Inventors: Joeri Beauprez, Zwijnaarde (BE); Pieter Coussement, Zwijnaarde (BE); Thomas Decoene, Zwijnaarde (BE); Annelies Vercauteren, Zwijnaarde (BE)

(21) Appl. No.: 18/555,649

(22) PCT Filed: Apr. 15, 2022
(86) PCT No.: PCT/EP2022/060180

§ 371 Date: Oct. 16, 2023

(30) Foreign Application Priority Data
Apr. 16, 2021 (EP) 21168995.5

(51) Int. Cl. C12P 19/00; C12N 9/10; C12N 9/24; C12P 17/06

(52) U.S. Cl. CPC C12P 19/00; C12N 9/1081; C12N 9/1085; C12N 9/2402; C12P 17/06; C12Y 205/01056; C12Y 302/01183

(57) ABSTRACT

The present invention is in the technical field of synthetic biology and metabolic engineering. More particularly, the present invention is in the technical field of metabolically engineered cells and use of said cells in a cultivation, preferably fermentation. The present invention describes a metabolically engineered cell and a method by cultivation, preferably fermentation, with said cell for production of a bioproduct. More specifically, the present invention describes a metabolically engineered cell and a method by cultivation, preferably fermentation, with said cell for production of an N-acetylneuraminic acid (Neu(n)Ac)-containing compound, wherein (n) is 4, 5, 7, 8 or 9 or a combination thereof. The metabolically engineered cell comprises a pathway for production of said Neu(n)Ac-containing compound and is modified in the expression or activity of at least one NeuNAc synthase according to the present invention. Furthermore, the present invention provides for purification of said Neu(n)Ac-containing compound from the cultivation.

### FIELD OF THE INVENTION

The present invention is in the technical field of synthetic biology and metabolic engineering. More particularly, the present invention is in the technical field of metabolically engineered cells and use of said cells in a cultivation, preferably a fermentation. The present invention describes a metabolically engineered cell and a method by cultivation, preferably fermentation, with said cell for production of a bioproduct. More specifically, the present invention describes a metabolically engineered cell and a method by cultivation, preferably fermentation, with said cell for production of an N-acetylneuraminic acid (Neu(n)Ac)-containing compound, wherein (n) is 4, 5, 7, 8 or 9 or a combination thereof. The metabolically engineered cell comprises a pathway for production of said Neu(n)Ac-containing compound and is modified in the expression or activity of at least one NeuNAc synthase according to the present invention. Furthermore, the present invention provides for purification of said Neu(n)Ac-containing compound from the cultivation.

### BACKGROUND

Bioproducts like e.g. N-acetylneuraminic acid (Neu(n)Ac)-containing or sialylated compounds comprising sialylated di- and oligosaccharides, glycoproteins and glycolipids, are involved in many vital phenomena such as development, differentiation, fertilization, embryogenesis, host pathogen adhesion and inflammation. Sialylated oligosaccharides can also be present as unconjugated glycans in body fluids and human milk wherein they modulate as bioactive glycans in important developmental and immunological processes (Bode, Early Hum. Dev. 2015, 91(11): 619-622; Bode, Nestle Nutr. Inst. Workshop Ser. 2019, 90: 191-201; Reily et al., Nat. Rev. Nephrol. 2019, 15: 346-366; Varki, Glycobiology 2017, 27: 3-49; Walsh et al., J. Funct. Foods 2020, 72: 10474). There is large scientific and commercial interest in bioproducts like e.g. sialylated compounds due to their wide functional spectrum. Yet, the availability of sialylated compounds like sialylated oligosaccharides is limited as production relies on chemical or chemo-enzymatic synthesis or on purification from natural sources such as e.g. animal milk. Chemical synthesis methods are laborious and time-consuming and because of the large number of steps involved they are difficult to scale-up. Enzymatic approaches using glycosyltransferases offer many advantages above chemical synthesis. Glycosyltransferases catalyse the transfer of a sugar moiety from an activated nucleotide-sugar donor onto saccharide or non-saccharide acceptors (Coutinho et al., J. Mol. Biol. 2003, 328: 307-317). These glycosyltransferases are the source for biotechnologists to synthesize bioproducts like e.g. Neu(n)Ac-containing compounds and are used both in (chemo)enzymatic approaches as well as in cell-based production systems. However, stereospecificity and regioselectivity of glycosyltransferases are still a formidable challenge. In addition, chemo-enzymatic approaches need to regenerate in situ nucleotide-sugar donors. Cellular production of bioproducts like e.g. Neu(n)Ac-containing compounds needs tight control of spatiotemporal availability of adequate levels of nucleotide-sugar donors in proximity of complementary glycosyltransferases. Due to these difficulties, current methods often result in small-scale synthesis of bioproducts like e.g. Neu(n)Ac-containing compounds.

PEP or phosphoenolpyruvate is a common precursor in the anabolism of a cell and of key importance for the synthesis of secondary metabolites such as flavonoids, aromatic amino acids and many monosaccharide subunits of di- and oligosaccharides or di- and oligosaccharide modifications. Such monosaccharide subunits are for instance Neu(n)Ac molecules, legionaminic acid, ketodeoxyoctonate, keto-deoxy-nonulonic acid, pseudaminic acid, N, N′-diacetyl-8-epilegionaminate, N-acetyl-D-muramate and their nucleotide and phosphorylated derivatives. To enhance synthesis of these bioproducts, the PEP concentration in the cell can be enhanced by means of overexpression and deletion of several genes.

Zhu et al. (Biotechnol. Lett. 2017, 39: 227-234) has shown that by the overexpression of PEP synthase (EC: 2.7.9.2) and PEP carboxykinase (EC: 4.1.1.49) the synthesis of N-acetylneuraminic acid was increased by 96,4% and 61% compared to the control respectively, combined overexpression increased the synthesis further up to 116,7% compared to the control. Zhu et al. (Biotechnol. Lett 2016, doi 10.1007/s10529-016-2215-z) has further shown that the deletion of a substrate phosphotransferase (PTS) system like the N-acetylglucosamine PTS system encoded by the gene nagE in E. coli, transporting and phosphorylating with the use of PEP N-acetylglucosamine (GIcNAc) and glucosamine (GIcN) into the cell, or like the mannose PTS system encoded by the genes manX, manYand manZin E. coli, transporting and phosphorylating with the use of PEP mannose, N-acetylmannosamine, glucose, fructose, GIcN and GIcNAc into the cell, increases Neu5Ac synthesis significantly. The upregulation of ppsA in E. coli was later also shown to be effective in EP3697805 and EP3575404, combining also ppsA overexpression with the deletion of manXYZ and nagE.

Zhang et al. (Biotech and Bioeng. 2018, 115(9): 217-2231) improved PEP synthesis in Bacillus subtilis in a similar fashion. The glucose PTS system was deleted to reduce PEP usages upon glucose uptake, the gene pyruvate kinase (EC: 2.7.1.40) was deleted to reduce PEP consumption and the gene PEP carboxykinase (EC: 4.1.1.49) was overexpressed to enhance the flux towards. To compensate for the deletion of the glucose PTS system, glucose permease and glucokinase were used to internalize and phosphorylate glucose in the cell. Further, the malic enzyme (EC: 1.1.1.38, EC: 1.1.1.39 or EC: 1.1.1.40) was introduced to increase the flux from the Krebs cycle towards pyruvate, the precursor of PEP. A reduced glycolysis and the introduction of the Entner-Doudoroff pathway further enhanced the production of N-acetylneuraminate. Note that these strains are in their basis modified in their acetate and lactate synthesis capacity, which inherently leads to improved availability of PEP, pyruvate and acetyl-CoA. Zhang et al. (Biotech. Adv. 2019, 37: 787-800) also reviewed and described how the precursors of N-acetylneuraminic acid and sialylated oligosaccharides can be modulated. By impacting the PEP and pyruvate availability in the cell, the flux towards sialylated oligosaccharides and N-acetylneuraminate (or other monosaccharide subunits as described above) is enhanced. Also here, techniques are described to delete or knock down the glycolysis pathway (comprising phosphofructokinase (pfkA gene, E.C.:2.7.1.11) and pyruvate kinase (pyk, EC: 2.7.1.40)) and to upregulate the phosphoenolpyruvate synthase gene (ppsA, EC: 2.7.9.2). Introduction or overexpression of the Entner-Doudoroff pathway and reduced PTS activity further led to improvements in synthesis. The system described was not only achieved by overexpression or deletions, but also by dynamic control through biosensors, which selectively upregulate and downregulate reactions in the cellular biochemistry.

### DESCRIPTION

### Summary of the Invention

It is an object of the present invention to provide for tools and methods by means of which a bioproduct, like e.g. a Neu(n)Ac-containing compound, can be produced by a cell and preferably in an efficient, time and cost-effective way which yields high amounts of the desired bioproduct.

According to the invention, this and other objects are achieved by providing a cell and a method for the production of a bioproduct. More specifically, the invention provides a cell and a method for the production of a Neu(n)Ac-containing compound wherein the cell is metabolically engineered, preferably has been metabolically engineered, with a pathway for the production of said Neu(n)Ac-containing compound and wherein the cell is modified, preferably has been modified, in the expression or activity of at least one Neu(n)Ac synthase as defined herein. Preferably, the cell comprises and expresses at least one glycosyltransferase involved in the production of said bioproduct.

The present invention provides a cell for the production of a bioproduct. More specifically, the present invention provides a cell for the production of a Neu(n)Ac-containing compound. It has now been found that the Neu(n)Ac synthases identified in the present invention provide for enzymes enabling fermentative production of a Neu(n)Ac-containing compound, and preferably having a positive effect, and even more preferably providing a better yield, productivity and/or specific productivity when used to metabolically engineer a cell producing said Neu(n)Ac-containing compound when compared to a cell with the same genetic background but lacking the Neu(n)Ac synthase identified in the present invention.

The present invention also provides a method for the production of a bioproduct. More specifically, the present invention also provides a method for the production of a Neu(n)Ac-containing compound. The method comprises the steps of providing a cell comprising a pathway for the production of a Neu(n)Ac-containing compound, wherein the cell is modified, preferably has been modified, in the expression or activity of at least one of said Neu(n)Ac synthases and preferably further comprises and expresses at least one glycosyltransferase involved in the production of said Neu(n)Ac-containing compound and cultivating said cell under conditions permissive to produce said sialylated di- and/or oligosaccharide. The present invention also provides methods to separate said Neu(n)Ac-containing compound.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of aspects of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the invention herein and within the scope of this invention, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verbs “to comprise”, “to have” and “to contain”, and their conjugations are used in their non-limiting sense to mean that items following the word are included, but items not specifically mentioned are not excluded. The verb “to consist essentially of” means that additional component(s) may be present than the ones specifically identified, said additional component(s) not altering the unique characteristic of the invention. Throughout the application and claims, unless specifically stated otherwise, the verbs “to comprise”, “to have” and “to contain”, and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the element is present, unless the context clearly requires that there is one and only one of the elements. The indefinite article “a” or “an” thus usually means “at least one”.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s). “Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins.

Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching.

Cyclic, branched and branched circular polypeptides may result from post-translational natural processes and may be made by entirely synthetic methods, as well. “Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source. “Recombinant” means genetically engineered DNA prepared by transplanting or splicing genes from one species into the cells of a host organism of a different species. Such DNA becomes part of the host's genetic makeup and is replicated.

The term “endogenous,” within the context of the present invention refers to any polynucleotide, polypeptide or protein sequence which is a natural part of a cell and is occurring at its natural location in the cell chromosome.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of said gene in any phase of the production process of the desired Neu(n)Ac-containing compound. Said modified expression is either a lower or higher expression compared to the wild type, wherein the term “higher expression” is also defined as “overexpression” of said gene in the case of an endogenous gene or “expression” in the case of a heterologous gene that is not present in the wild type strain. Lower expression is obtained by means of common well-known technologies for a skilled person (such as the usage of siRNA, CrispR, CrispRi, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. Overexpression or expression is obtained by means of common well-known technologies for a skilled person, wherein said gene is part of an “expression cassette” which relates to any sequence in which a promoter sequence, untranslated region sequence (containing either a ribosome binding sequence or Kozak sequence), a coding sequence (for instance a Neu(n)Ac synthase gene sequence) and optionally a transcription terminator is present, and leading to the expression of a functional active protein. Said expression is either constitutive or conditional or regulated or tuneable.

The term “constitutive expression” is defined as expression that is not regulated by transcription factors other than the subunits of RNA polymerase (e.g. the bacterial sigma factors) under certain growth conditions. Non-limiting examples of such transcription factors are CRP, Lacl, ArcA, Cra, IcIR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions.

RNA polymerase binds a specific sequence to initiate transcription, for instance via a sigma factor in prokaryotic hosts.

The term “regulated expression” is defined as expression that is regulated by transcription factors other than the subunits of RNA polymerase (e.g. bacterial sigma factors) under certain growth conditions.

Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified activity” of a protein relates to a non-native activity of said protein in any phase of the production process of the desired bioproduct. The term “non-native”, as used herein with reference to the activity of a protein indicates that the protein has been modified to have an abolished, impaired, reduced, delayed, higher, accelerated or improved activity compared to the native activity of said protein.

The term “non-native”, as used herein with reference to a cell producing a Neu(n)Ac-containing compound, indicates that the Neu(n)Ac-containing compound is i) not naturally produced or ii) when naturally produced not in the same amounts by the cell; and that the cell has been genetically modified to be able to produce said Neu(n)Ac-containing compound or to have a higher production of the Neu(n)Ac-containing compound.

The term “modified expression or activity of a Neu(n)Ac synthase” as used herein refers to i) higher expression or overexpression of an endogenous Neu(n)Ac synthase, ii) expression of a heterologous Neu(n)Ac synthase or iii) expression and/or overexpression of a mutant Neu(n)Ac synthase that has a higher Neu(n)Ac synthase activity compared to the wild-type (i.e. native) Neu(n)Ac synthase protein. “Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination.

A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present invention contemplates making functional variants by modifying the structure of a Neu(n)Ac synthase as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the invention results in a functional homolog can be readily determined by assessing the ability of the variant polypeptide to produce a response in cells in a fashion similar to the wild-type polypeptide.

In the context of the present invention, a preferred “variant” of a polypeptide (usually indicated with a SEQ ID NO) is a functional fragment of said polypeptide, i.e. said fragment retains the functional characteristic of said polypeptide. If the polypeptide is a Neu(n)Ac synthase as described herein, then a functional fragment thereof retains the functional characteristic to produce Neu(n)Ac. If the polypeptide is a N-acylneuraminate-9-phosphate synthetase, then a functional fragment thereof retains the functional characteristic to produce N-acylneuraminate-9-phosphate. A “fragment” of a polypeptide (usually indicated with a SEQ ID NO) as used herein preferably means a polypeptide sequence which comprises or consists of an amount of consecutive amino acid residues from said polypeptide SEQ ID NO and wherein said amount of consecutive amino acid residues is preferably at least 50%, 60.0%, 70.0%, 80.0%, 81.0 %, 82.0%, 83.0%, 84.0%, 85.0%, 86.0%, 87.0%, 88.0%, 89.0%, 90%, 91%, 92%, 93%, 94%, 95%, 95.5%, 96%, 96.5%, 97%, 97.5%, 98%, 98.5%, 99%, 99.5%, preferably at least 80.0%, more preferably at least 87.0%, even more preferably at least 90.0%, even more preferably at least 95.0%, most preferably at least 97.0% of the full-length of said polypeptide SEQ ID NO and which performs at least one biological function of the intact polypeptide (i.e. production of Neu(n)Ac when said polypeptide is a Neu(n)Ac synthase; production of N-acylneuraminate-9-phsoophate when said polypeptide is a N-acylneuraminate-9-phosphate synthetase) in substantially the same manner, preferably to a similar or greater extent, as does the intact polypeptide which can be routinely assessed by the skilled person. As such, a “fragment” of a polypeptide (usually indicated with a SEQ ID NO) as used herein preferably means a polypeptide sequence which comprises or consists of said polypeptide SEQ ID NO, wherein an amount of consecutive amino acid residues is missing and wherein said amount is no more than 50.0%, 40.0%, 30.0% of the full-length of said polypeptide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, m ore preferably no more than 15.0%, even more preferably no more than 10.0%, even more preferably no more than 5.0%, most preferably no more than 2.5%, of the full-length of said polypeptide SEQ ID NO and which performs at least one biological function of the intact polypeptide (i.e. production of Neu(n)Ac when said polypeptide is a Neu(n)Ac synthase; production of N-acylneuraminate-9-phosphate when said polypeptide is a N-acylneuraminate-9-phosphate synthetase) in substantially the same manner, preferably to a similar or greater extent, as does the intact polypeptide which can be routinely assessed by the skilled person.

Throughout the application and claims, the phrase “variant or derivative” is preferably replaced with “functional fragment”.

The term “functional homolog” as used herein describes those molecules that have sequence similarity and also share at least one functional characteristic such as a biochemical activity. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514). In the context of the present invention, a functional homolog of a Neu(n)Ac synthase “Y” according to the invention (usually indicated with a SEQ ID NO) refers to a Neu(n)Ac synthase which has Neu(n)Ac synthase activity, i.e. the functional homolog retains the functional characteristic of Neu(n)Ac synthase “Y” to produce Neu(n)Ac. In the context of the present invention, a functional homolog of a N-acylneuraminate-9-phosphate synthetase “Z” according to the invention (usually indicated with a SEQ ID NO) refers to a N-acylneuraminate-9-phosphate synthetase which has N-acylneuraminate-9-phosphate synthetase activity, i.e. the functional homolog retains the functional characteristic of N-acylneuraminate-9-phosphate synthetase “Z” to produce N-acylneuraminate-9-phosphate.

Functional homologs are sometimes referred to as orthologs, where “ortholog” refers to a homologous gene or protein that is the functional equivalent of the referenced gene or protein in another species.

Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme.

Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (EI-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48(D1) (2020) D606-D612). It should be understood for those skilled in the art that for the databases used herein, comprising Pfam 32.0 (released Sept 2018), CDD v3.17 (released Apr. 3, 2019), eggnogdb 4.5.1 (released Sept 2016), InterPro 75.0 (released Jul 4, 2019), TCDB (released Jun. 17, 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art. The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like for example BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: https://blast.ncbi.nlm.nih.gov/Blast.cgi.

Clustal Omega (Clustal Ω) is a multiple sequence alignment program that uses seeded guide trees and HMM profile-profile techniques to generate alignments between three or more sequences. It produces biologically meaningful multiple sequence alignments of divergent sequences. The web interface for Clustal Q is available at https://www.ebi.ac.uk/Tools/msa/clustalo/. Default parameters for multiple sequence alignments and calculation of percent identity of protein sequences using the Clustal Q method are: enabling de-alignment of input sequences: FALSE; enabling mbed-like clustering guide-tree: TRUE; enabling mbed-like clustering iteration: TRUE; Number of (combined guide-tree/HMM) iterations: default(0); Max Guide Tree Iterations: default [-1]; Max HMM Iterations: default [-1]; order: aligned. MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g. BLOSUM50, BLOSUM62, and PAM250) to employ with their protein sequence examination. EMBOSS Needle (https://galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having/comprising an amino acid sequence having at least 80% overall sequence identity (or a protein sequence having at least 80% overall sequence identity) to the full-length sequence of a reference polypeptide sequence is to be understood as that the sequence has 80%, 81%, 82%, 83%, 84%, 85%, 86%, 87%,88%, 89%, 90%, 91%, 92%, 93%,94%, 95%, 96%, 97%, 98%, 99 %, 100% overall sequence identity to the full-length of the amino acid sequence of the reference polypeptide sequence. Throughout the application, unless explicitly specified otherwise, a polypeptide comprising/consisting/having/represented by an amino acid sequence having at least 80% sequence overall sequence identity to the full-length amino acid sequence of a reference polypeptide, usually indicated with a SEQ ID NO, preferably has at least 85%, 90%, 91%, 92%, 93%, 94%, 95%, 96%, 97%, 98% or 99%, more preferably has at least 85%, even more preferably has at least 90%, even more preferably has at least 95%, even more preferably has at least 97%,most preferably at least 99%, overall sequence identity to the full length reference sequence. In the context of the present invention, a polypeptide having an amino acid sequence having e.g. at least 80% overall sequence identity (or a protein sequence having e.g. at least 80% overall sequence identity) to the full-length sequence of a reference Neu(n)Ac synthase ‘Y’ or reference N-acylneuraminate-9-phosphate synthetase ‘Z’ (usually indicated with a SEQ ID NO) refers to a polypeptide which is able to produce Neu(n)Ac or N-acylneuraminate-9-phosphate, respectively, as described herein, i.e. the polypeptide retains the functional characteristic of the reference neu(n)Ac synthase ‘Y’ to produce neu(n)Ac or of the reference N-acylneuraminate-9-phosphate synthetase ‘Z’ to produce N-acylneuraminate-9-phosphate.

For the purpose of this invention, the overall sequence identity of a polypeptide is preferably determined by the program EMBOSS Needle 5.0 (https://galaxy-iuc.github.io/emboss-5.0-docs/needle.html), preferably with default parameters (the substitution matrix EBLOSUM62, the gap opening penalty 10.0, and the gap extension penalty 0.5) and preferably with sequences of mature proteins (i.e. without taking into account secretion signals or transit peptides).

The term “bioproduct” as used herein refers to the group of molecules comprising monosaccharide, phosphorylated monosaccharide, activated monosaccharide, disaccharide, oligosaccharide, aglycon, glycolipid and glycoprotein.

The term “Neu(n)Ac-containing compound” as used herein refers to Neu(n)Ac as defined herein as well as to a compound comprising a disaccharide, an oligosaccharide, a glycolipid and a glycoprotein that comprises one or more Neu(n)Ac molecules.

The terms “sialic acid”, “Neu(n)Ac (wherein (n) is 4, 5, 7, 8 or 9 or a combination thereof)”, “N-acetylneuraminate”, “N-acylneuraminate”, “N-acetylneuraminic acid”, “Neu(n)Ac molecule” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4 and Neu4,5,7,8,9Ac5 and Neu5Gc. Neu4Ac is also known as 4-O-acetyl-5-amino-3,5-dideoxy-D-glycero-D-galacto-non-2-ulopyranosonic acid or 4-O-acetyl neuraminic acid and has C11H19NO9 as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C13H21NO10 as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C13H21NO10 as molecular formula. Neu5,8Ac2 is also known as 5-n-acetyl-8-o-acetyl neuraminic acid and has C13H21NO10 as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C13H21NO10 as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetyneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolyl-neuraminic acid, N-glycolylneuraminicacid, N-glycolylneuraminate, N-glycoloyl-neuraminate, N-glycoloyl-neuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C11H19NO10 as molecular formula.

The terms “Neu(n)Ac synthase”, “N-acetylneuraminic acid synthase”, “N-acetylneuraminate synthase”, “sialic acid synthase”, “NeuAc synthase”, “NeuB”, “NeuB1”, “Neu(n)Ac synthase”, “NANA condensing enzyme”, “N-acetylneuraminate lyase synthase”, “N-acetylneuraminic acid condensing enzyme” as used herein are used interchangeably and refer to an enzyme capable to synthesize sialic acid (Neu(n)Ac) from N-acetylmannosamine (ManNAc) in a reaction using phosphoenolpyruvate (PEP).

The terms “CMP-sialic acid synthase”, “N-acylneuraminate cytidylyltransferase”, “CMP-sialate synthase”, “CMP-Neu(n)Ac synthase”, “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “N-acylneuraminate-9-phosphate synthetase”, “NANA synthase”, “NANAS”, “NANS”, “NmeNANAS”, “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesize N-acylneuraminate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase and N-acyl-D-glucosamine 2-epimerase.

An UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GIcNAc-2-epimerase and UDP-N-acetyl-D-glucosamine 2-epimerase.

An N-acetylmannosamine-6-phosphate 2-epimerase is an enzyme that catalyses the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

The terms “N-acetylneuraminate Iyase”, “Neu5Ac Iyase”, “N-acetylneuraminate pyruvate-Iyase”, “N-acetylneuraminic acid aldolase”, “NALase”, “sialate lyase”, “sialic acid aldolase”, “sialic acid lyase” and “nanA” are used interchangeably and refer to an enzyme that degrades N-acetylneuraminate into N-acetylmannosamine (ManNAc) and pyruvate.

The terms “N-acetylneuraminate kinase”, “ManNAc kinase”, “N-acetyl-D-mannosamine kinase” and “nanK” are used interchangeably and refer to an enzyme that phosphorylates ManNAc to synthesize N-acetylmannosamine-phosphate (ManNAc-6-P).

The terms “ManNAc-6-P isomerase”, “ManNAc-6-P 2-epimerase” and “nanE” are used interchangeably and refer to an enzyme that converts ManNAc-6-P to N-acetylglucosamine-6-phosphate (GIcNAc-6-P).

The terms “N-acetylglucosamine-6-P deacetylase” and “nagA” are used interchangeably and refer to an enzyme that catalyses the hydrolysis of the N-acetyl group of N-acetylglucosamine-6-phosphate (GIcNAc-6-P) to yield glucosamine-6-phosphate (GIcN6P) and acetate.

The terms “glucosamine-6-P deaminase”, “GIcN6P deaminase”, “glucosamine-6-phosphate isomerase”, “glmD” and “nagB” are used interchangeably and refer to an enzyme that catalyses the reversible isomerization-deamination of glucosamine-6-phosphate (GIcN6P) to form fructose-6-phosphate and an ammonium ion.

The term “glycosyltransferase” as used herein refers to an enzyme capable to catalyse the transfer of sugar moieties from activated donor molecules to specific acceptor molecules, forming glycosidic bonds.

A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases. Fucosyltransferases are glycosyltransferases that transfer a fucose residue (Fuc) from a GDP-fucose (GDP-Fuc) donor onto an acceptor. Fucosyltransferases comprise alpha-1,2-fucosyltransferases, alpha-1,3-fucosyltransferases, alpha-1,4-fucosyltransferases and alpha-1,6-fucosyltransferases that catalyse the transfer of a Fuc residue from GDP-Fuc onto an acceptor via alpha-glycosidic bonds. Fucosyltransferases can be found but are not limited to the GT10, GT11, GT23, GT65 and GT68 CAZy families. Sialyltransferases are glycosyltransferases that transfer a sialic acid (like Neu5Ac or Neu5Gc) from a donor (like CMP-Neu5Ac or CMP-Neu5Gc) onto an acceptor. Sialyltransferases comprise alpha-2,3-sialyltransferases, alpha-2,6-sialyltransferases and alpha-2,8-sialyltransferases that catalyse the transfer of a sialic acid onto an acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto an acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto an acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto an acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto an acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto an acceptor. Mannosyltransferases comprise alpha-1,2-mannosyltransferases, alpha-1,3-mannosyltransferases and alpha-1,6-mannosyltransferases that transfer a Man residue from GDP-Man onto an acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GIcNAc) from an UDP-N-acetylglucosamine (UDP-GIcNAc) donor onto an acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GIcNAc from an UDP-GIcNAc donor onto a terminal galactose unit present in an acceptor via a beta-1,3-linkage. Beta-1,6-N-acetylglucosaminyltransferases are N-acetylglucosaminyltransferases that transfer GIcNAc from an UDP-GIcNAc donor onto an acceptor via a beta-1,6-linkage. N-acetylgalactosaminyltransferases are glycosyltransferases that transfer an N-acetylgalactosamine group (GaINAc) from an UDP-N-acetylgalactosamine (UDP-GaINAc) donor onto an acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GaINAc from an UDP-GaINAc donor to an acceptor via an alpha-1,3-linkage. N-acetylmannosaminyltransferases are glycosyltransferases that transfer an N-acetylmannosamine group (ManNAc) from an UDP-N-acetylmannosamine (UDP-ManNAc) donor onto an acceptor. Xylosyltransferases are glycosyltransferases that transfer a xylose residue (Xyl) from an UDP-xylose (UDP-Xyl) donor onto an acceptor. Xylosyltransferases can be found but are not limited to GT14, GT61 and GT77 CAZy families. Glucuronyltransferases are glycosyltransferases that transfer a glucuronate from an UDP-glucuronate donor onto an acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto an acceptor. N-glycolylneuraminyltransferases are glycosyltransferases that transfer an N-glycolylneuraminic acid group (Neu5Gc) from a CMP-Neu5Gc donor onto an acceptor. Rhamnosyltransferases are glycosyltransferases that transfer a rhamnose residue from a GDP-rhamnose donor onto an acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto an acceptor. UDP-4-amino-4,6-dideoxy-N-acetyl-beta-L-altrosamine transaminases are glycosyltransferases that use an UDP-2-acetamido-2,6-dideoxy—L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or an UDP-N-acetylfucosamine donor onto an acceptor.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-ldopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy—L-arabino-4-hexulose, 2-acetamido-2,6-dideoxy—L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GIcNAc), glucosamine (Glcn), mannose (Man), xylose (Xyl), N-acetylmannosamine (ManNAc), N-glycolylneuraminic acid, N-acetylgalactosamine (GaINAc), galactosamine (Galn), fucose (Fuc), rhamnose (Rha), glucuronic acid, gluconic acid, fructose (Fru) and polyols.

The term “phosphorylated monosaccharide” as used herein refers to one of the above listed monosaccharides which is phosphorylated. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GIcNAc), UDP-N-acetylgalactosamine (UDP-GaINAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy—L-arabino-4-hexulose, UDP-2-acetamido-2,6-dideoxy—L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “disaccharide” as used herein refers to a saccharide polymer containing two simple sugars, i.e. monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GIcNAc), N-acetyllactosamine (Gal-b1,4-GIcNAc), LacDiNAc (GaINAc-b1,4-GIcNAc), N-acetylgalactosaminylglucose (GaINAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to twenty, of simple sugars, i.e. monosaccharides. Preferably the oligosaccharide as described herein contains monosaccharides selected from the list as used herein above. The oligosaccharide as used in the present invention can be a linear structure or can include branches. The linkage (e.g. glycosidic linkage, galactosidic linkage, glucosidic linkage, etc.) between two sugar units can be expressed, for example, as 1,4, 1->4, or (1-4), used interchangeably herein. Each monosaccharide can be in the cyclic form (e.g. pyranose or furanose form).

An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides (MMOs), 0-antigen, , the glycan chain present in lipopolysaccharides (LPS), the oligosaccharide repeats present in capsular polysaccharides, peptidoglycan (PG) and amino-sugars.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose Ill, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose Ill, isomeric fucosylated lacto-N-hexaose Ill, isomeric fucosylated lacto-N-hexaose 1, sialyllacto-N-hexaose, sialyllacto-N-neohexaose 11, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF 1), Lacto-N-fucopentaose II (LNF 11), Lacto-N-fucopentaose 11 (LNF 111), lacto-N-fucopentaose V (LNF V), lacto-N-fucopentaose VI (LNF VI), lacto-N-neofucopentaose 1, lacto-N-difucohexaose I (LDFH 1), lacto-N-difucohexaose II (LDFH 11), Monofucosyllacto-N-hexaose 11 (MFLNH 111), Difucosyllacto-N-hexaose (DFLNHa), difucosyl-lacto-N-neohexaose.

As used herein, a ‘sialylated oligosaccharide’ is to be understood as a charged sialic acid containing oligosaccharide, i.e. an oligosaccharide having a sialic acid residue. It has an acidic nature. Some examples are 3-SL (3′-sialyllactose or 3′SL or Neu5Ac-a2,3-Gal-b1,4-Glc), 3′-sialyllactosamine, 6-SL (6′-sialyllactose or 6′SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Aca-2,3GalI-1,3GalNacp-1,3Gala-1,4Galp-1,4Gal), sialylated tetrasaccharide (Neu5Aca-2,3Ga1β-1,4GlcNacp-14GIcNAc), pentasaccharide LSTD (Neu5Aca-2,3Galp-1,4GlcNacp-1,3Galp-1,4Glc), sialylated lacto-N-triose, sialylated lacto-N-tetraose, sialyllacto-N-neotetraose, monosialyllacto-N-hexaose, disialyllacto-N-hexaose 1, monosialyllacto-N-neohexaose 1, monosialyllacto-N-neohexaose 11, disialyllacto-N-neohexaose, disialyllacto-N-tetraose, disialyllacto-N-hexaose 11, sialyllacto-N-tetraose a, disialyllacto-N-hexaose 1, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose 11, disialyllacto-N-fucopentaose 11, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Acα-2,3Ga1β-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Ga1β-1,4Glc, GM1 Galβ-1,3GalNAcβ-1,4(Neu5Acα-2,3)Ga1β-1,4Glc, GD1a Neu5Acα-2,3Galβ-1,3GaINAcβ-1,4(Neu5Acα-2,3)Galβ-1,4Glc, GT1a Neu5Acα-2,8Neu5Acα-2,3Ga1β-1,3GaINAcβ-1,4(Neu5Acα-2,3)Galβ-1,4Glc, GD2 GalNAcβ-1,4(Neu5Acα-2,8Neu5Acα2,3)Ga1β-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Ga1β-1,4Glc, GT1b Neu5Acα-2,3Galβ-1,3GaINAcβ-1,4(Neu5Acα-2,8Neu5Acα2,3)Galβ-1,4Glc, GQ1b Neu5Acα-2,8Neu5Acα-2,3Galβ-1,3GaINAc 3-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GaINAc 1-1,4(Neu5Acα-2,8Neu5Acα-2,8Neu5Acα2,3)Galβ-1,4Glc, GP1c Neu5Acα-2,8Neu5Acα-2,3Ga1β-1,3GaINAc 1-1,4(Neu5Acα-2,8Neu5Acα-2,8Neu5Acα2,3)Galβ-1,4Glc, GD1a Neu5Acα-2,3Galβ-1,3(Neu5Acα-2,6)GaINAcβ-1,4Galβ-1,4Glc, Fucosyl-GM1 Fuca-1,2Galβ-1,3GalNAcβ-1,4(Neu5Acα-2,3)Gal P-1,4Glc; all of which may be extended to the production of the corresponding gangliosides by reacting the above oligosaccharide moieties with ceramide or synthetizing the above oligosaccharides on a ceramide.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose Ill, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides comprise oligosaccharides present in milk found in any phase during lactation including colostrum milk from humans and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus).

As used herein the term “Lewis-type antigens” comprise the following oligosaccharides: H1 antigen, which is Fucα1-2Galβ1-3GIcNAc, or in short 2′FLNB; Lewisa, which is the trisaccharide Gal 1-3[Fucα1-4]GIcNAc, or in short 4-FLNB; Lewisb, which is the tetrasaccharide Fucα1-2Galβ1-3[Fucα1-4]GIcNAc, or in short DiF-LNB; sialyl Lewisa which is 5-acetylneuraminyl-(2-3)-galactosyl-(1-3)-(fucopyranosyl-(1-4))-N-acetylglucosamine, or written in short Neu5Acα2-3Gal1-3[Fucα1-4]GIcNAc; H2 antigen, which is Fucα1-2Galβ1-4GIcNAc, or otherwise stated 2′fucosyl-N-acetyl-lactosamine, in short 2′FLacNAc; Lewisx, which is the trisaccharide Gal 1-4[Fucα1-3]GIcNAc, or otherwise known as 3-Fucosyl-N-acetyl-lactosamine, in short 3-FLacNAc, Lewisy, which is the tetrasaccharide Fucα1-2Galβ1-4[Fucα1-3]GIcNAc and sialyl Lewisx which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GIcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria which are composed of a lipid A, a core oligosaccharide and the O-antigen. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group.

The terms “LNT II”, “LNT-II”, “LN3”, “lacto-N-triose II”, “lacto-N-triose II”, “lacto-N-triose”, “lacto-N-triose” or “GlcNAc(31-3Gal(31-4Glc” as used in the present invention, are used interchangeably.

The terms “LNT”, “lacto-N-tetraose”, “lacto-N-tetraose” or “Gal 31-3GlcNAc(31-3Galβ1-4Glc” as used in the present invention, are used interchangeably.

The terms “LNnT”, “lacto-N-neotetraose”, “lacto-N-neotetraose”, “neo-LNT” or “Galβ1-4GlcNAc31-3Galβ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GIcNAc-b1,3-Gal-b1,4-Glc” as used in the present invention, are used interchangeably.

The terms “LSTb”, “LS-Tetrasaccharide b”, “Sialyl-lacto-N-tetraose b”, “sialyllacto-N-tetraose b” or “Gal-b1,3-(Neu5Ac-a2,6)-GIcNAc-b1,3-Gal-b1,4-Glc” as used in the present invention, are used interchangeably.

The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-a2,6-Gal-b1,4-GIcNAc-b1,3-Gal-b1,4-Glc” as used in the present invention, are used interchangeably.

The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-a2,3-Gal-b1,4-GIcNAc-b1,3-Gal-b1,4-Glc” as used in the present invention, are used interchangeably.

The term “aglycon” refers to the noncarbohydrate group of a glycoside. As used herein, the term “glycolipid” refers to any of the glycolipids which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like for example glycerol (glycoglycerolipids), peptide (glycopeptidolipids), acylated-sphingosine (glycosphingolipids), or other residues (lipopolysaccharides, phenolic glycolipids, nucleoside lipids). Examples of Neu(n)Ac-containing glycolipids comprise octyl-beta-sialyllactoside, sialoglycosphingolipids and gangliosides.

The term “glycoprotein” refers to any of the glycoproteins which are generally known in the art. Glycoproteins can be subclassified based on the type of glycosylation present on the amino acid residues of the glycoprotein into N-glycosylated, 0-glycosylated, P-glycosylated, C-glycosylated and S-glycosylated proteins. Examples of Neu(n)Ac-containing glycoproteins comprise but are not limited to sialyl-Tn-MUC1 and sialyl-T-MUC1 glycopeptides containing Neu5Gc, sialoglycopolypeptides, sialoglycoproteins, glycophorins like e.g. glycophorin A and glycophorin C, podocalyxin, gonadotropin receptors, podoplanin, CD43 (leukosialin, sialophorin) and the prion protein PrP.

The term “pathway for production of a bioproduct” as used herein is a biochemical pathway consisting of the enzymes and their respective genes involved in the synthesis of a bioproduct as defined herein. The term “pathway for production of a Neu(n)Ac-containing compound” as used herein is a biochemical pathway consisting of the enzymes and their respective genes involved in the synthesis of a Neu(n)Ac-containing compound as defined herein. Said pathway for production of a Neu(n)Ac-containing compound comprises at least one Neu(n)Ac synthase. Furthermore, said pathway for production of a Neu(n)Ac-containing compound can comprise but is not limited to pathways involved in the synthesis of a nucleotide-activated sugar and the transfer of said nucleotide-activated sugar to an acceptor to create a Neu(n)Ac-containing compound of the present invention. Further examples of such pathway comprise but are not limited to a fucosylation, sialylation, galactosylation, N-acetylglucosaminylation, N-acetylgalactosylation, mannosylation, N-acetylmannosaminylation pathway.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to α 1,2; α 1,3; α 1,4 and/or α 1,6 fucosylated compounds.

A ‘sialylation pathway’ is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, N-acylneuraminate-9-phosphate phosphatase, Neu(n)Ac synthase and CMP sialic acid synthase combined with a sialyltransferase leading to α 2,3; α 2,6 and/or α 2,8 sialylated compounds.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase combined with a galactosyltransferase leading to a galactosylated compound comprising a mono-, di-, or oligosaccharide having an alpha or beta bound galactose on any one or more of the 2, 3, 4 and 6 hydroxyl group of said mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase combined with a glycosyltransferase leading to a GIcNAc-modified compound comprising a mono-, di-, or oligosaccharide having an alpha or beta bound N-acetylglucosamine (GIcNAc) on any one or more of the 3, 4 and 6 hydroxyl group of said mono-, di- or oligosaccharide.

An ‘N-acetylgalactosylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, UDP-N-acetylglucosamine 4-epimerase, UDP-galactose 4-epimerase, N-acetylgalactosamine kinase and/or UDP-GalNAc pyrophosphorylase combined with a glycosyltransferase leading to a GaINAc-modified compound comprising a mono-, di- or oligosaccharide having an alpha or beta bound N-acetylgalactosamine on said mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanyltransferase combined with a glycosyltransferase leading to a mannosylated compound comprising a mono-, di- or oligosaccharide having an alpha or beta bound mannose on said mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GIcNAc 2-epimerase and/or ManNAc kinase combined with a glycosyltransferase leading to a ManNAc-modified compound comprising a mono-, di- or oligosaccharide having an alpha or beta bound N-acetylmannosamine on said mono-, di- or oligosaccharide.

The term “enabled efflux” means to introduce the activity of transport of a solute over the cytoplasm membrane and/or the cell wall. Said transport may be enabled by introducing and/or increasing the expression of a transporter protein as described in the present invention. The term “enhanced efflux” means to improve the activity of transport of a solute over the cytoplasm membrane and/or the cell wall.

Said transport may be enhanced by introducing and/or increasing the expression of a transporter protein as described in the present invention. “Expression” of a transporter protein is defined as “overexpression” of the gene encoding said transporter protein in the case said gene is an endogenous gene or “expression” in the case the gene encoding said transporter protein is a heterologous gene that is not present in the wild type strain or cell.

The terms “acetyl-coenzyme A synthetase”, “acs”, “AcCoA synthetase”, “acetat--CoA ligase”, “acyl-activating enzyme” and “yfaC” are used interchangeably and refer to an enzyme that catalyses the conversion of acetate into acetyl-coezyme A (AcCoA) in an ATP-dependent reaction.

The terms “pyruvate dehydrogenase”, “pyruvate oxidase”, “POX”, “poxB” and “pyruvate:ubiquinone-8 oxidoreductase” are used interchangeably and refer to an enzyme that catalyses the oxidative decarboxylation of pyruvate to produce acetate and C02.

The terms “lactate dehydrogenase”, “D-lactate dehydrogenase”, “IdhA”, “hsll”, “htpH”, “D-LDH”, “fermentative lactate dehydrogenase” and “D-specific 2-hydroxyacid dehydrogenase” are used interchangeably and refer to an enzyme that catalyses the conversion of lactate into pyruvate hereby generating NADH.

As used herein, the term “cell productivity index (CPI)” refers to the mass of the bioproduct produced by the cells divided by the mass of the cells produced in the culture.

The term “purified” refers to material that is substantially or essentially free from components which interfere with the activity of the biological molecule. For cells, saccharides, nucleic acids, and polypeptides, the term “purified” refers to material that is substantially or essentially free from components which normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the bioproduct, like e.g. the Neu(n)Ac-containing compound, that is produced by the cell in whole broth, i.e. inside (intracellularly) as well as outside (extracellularly) of the cell.

The term “precursor” as used herein refers to substances which are taken up or synthetized by the cell for the specific production of a bioproduct like e.g. a Neu(n)Ac-containing compound according to the present invention. In this sense a precursor can be an acceptor as defined herein, but can also be another substance, metabolite, which is first modified within the cell as part of the biochemical synthesis route of the bioproduct like e.g. a Neu(n)Ac-containing compound. Examples of such precursors comprise the acceptors as defined herein, and/or glucose, galactose, fructose, glycerol, sialic acid, fucose, mannose, maltose, sucrose, lactose, glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetyl-Neuraminic acid-9-phosphate, CMP-sialic acid, mannose-6-phosphate, mannose-1-phosphate, GDP-mannose, GDP-4-dehydro-6-deoxy-α-D-mannose, and/or GDP-fucose.

Optionally, the cell is transformed to comprise and to express at least one nucleic acid sequence encoding a protein selected from the group consisting of lactose transporter, N-acetylneuraminic acid transporter, fucose transporter, glucose transporter, galactose transporter, transporter for a nucleotide-activated sugar wherein said transporter internalizes a to the medium added precursor for the synthesis of the bioproduct like e.g. a Neu(n)Ac-containing compound of present invention.

The term “acceptor” as used herein refers to a mono-, di- or oligosaccharide, a lipid or protein which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GIcNAc-Asn residues, glycoproteins with terminal GIcNAc and Gal residues, immunoglobulins.

### DETAILED DESCRIPTION OF THE INVENTION

According to a first aspect, the present invention provides a metabolically engineered cell for the production of a bioproduct of the list comprising monosaccharide, activated monosaccharide, phosphorylated monosaccharide, disaccharide, oligosaccharide, aglycon, glycolipid or glycoprotein. Herein, a metabolically engineered cell comprising a pathway for the production of said bioproduct is provided. Examples of such pathways comprise but are not limited to pathways involved in the synthesis of monosaccharide, phosphorylated monosaccharide, nucleotide-activated sugar, lipid and/or protein and/or glycosylation pathways like e.g. a fucosylation, sialylation, galactosylation, N-acetylglucosaminylation, N-acetylgalactosylation, mannosylation and/or N-acetylmannosaminylation pathway. Said pathway for the production of a bioproduct comprising a disaccharide, oligosaccharide, aglycon, glycolipid or glycoprotein preferably comprises at least one glycosyltransferase that is involved in the production of said bioproduct.

In a preferred embodiment of the method and/or cell of present invention, the cell comprises one or more pathway(s) for monosaccharide synthesis. Said pathways for monosaccharide synthesis comprise enzymes like e.g. carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like e.g. UMP and UDP, respectively, and enzymes involved in the synthesis of phosphoenolpyruvate (PEP).

In another and/or additional preferred embodiment of the method and/or cell of present invention, the cell comprises one or more pathway(s) for phosphorylated monosaccharide synthesis. Said pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like e.g. but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of present invention, the cell comprises one or more pathways for the synthesis of one or more nucleotide-activated sugars. Said pathways for nucleotide-activated sugar synthesis comprise enzymes like e.g. PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, glucophosphomutase and/or N-acetylglucosamine-1-phosphate uridylyltransferase.

In another and/or additional preferred embodiment, the cell comprises at least one glycosyltransferase that is involved in the production of said bioproduct. Said glycosyltransferase can be chosen from the list comprising but not limited to fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

According to a second aspect, the present invention provides a metabolically engineered cell for the production of an N-acetylneuraminic acid (Neu(n)Ac)-containing compound wherein (n) is 4, 5, 7, 8 or 9 or a combination thereof. Herein, a metabolically engineered microbial host cell comprising a pathway for the production of a Neu(n)Ac-containing compound is provided which expresses at least one Neu(n)Ac synthase which has Neu(n)Ac synthase activity and which has PFAM domain PF03102 and/or PatricDB global family domain PGF_06907304 and which comprises the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid, or which comprises a polypeptide sequence according to any one of SEQ ID NO 02, 03, 04, 05, 06, 07, 08, 09, 10, 11, 12, 13, 14, 15, 16, 17 or 18, or which is a functional homolog, variant or derivative of any one of SEQ ID NO 02, 03, 04, 05, 06, 07, 08, 09, 10, 11, 12, 13, 14, 15, 16, 17 or 18 having at least 80% overall sequence identity to the full-length of any one of said polypeptide with SEQ ID NO 02, 03, 04, 05, 06, 07, 08, 09, 10, 11, 12, 13, 14, 15, 16, 17 or 18 and having Neu(n)Ac synthase activity, respectively. Said cell may further comprise and express at least one glycosyltransferase that is involved in the production of said Neu(n)Ac-containing compound.

Throughout the application, unless specifically stated otherwise, the expression “polypeptide sequence according to any one of SEQ ID NO 02, 03, 04, 05, 06, 07, 08, 09, 10, 11, 12, 13, 14, 15, 16, 17 or 18” is preferably replaced by the expression “polypeptide sequence according to any one of SEQ ID NO 02, 04, 05, 06, 07, 08, 09, 10, 11, 12, 13, 14, 15, 16, 17 or 18”. In other words, a Neu(n)Ac synthase according to the invention preferably does not comprise SEQ ID NO 03 and/or is preferably not a functional homolog, variant or derivative of SEQ ID NO 03.

According to a third aspect, the present invention provides a method for the production of a bioproduct, like e.g. an N-acetylneuraminic acid (Neu(n)Ac)-containing compound wherein (n) is 4, 5, 7, 8 or 9 or a combination thereof as described herein, by a metabolically engineered cell. The method comprises the steps of:
1) providing a cell as described herein, and
2) cultivating said cell under conditions permissive to produce said bioproduct like e.g. a Neu(n)Ac-containing compound.

Preferably, the bioproduct like e.g. the Neu(n)Ac-containing compound is separated from the cultivation as explained herein.

In the scope of the present invention, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3. Preferably, said pH range is 2-10.

The present invention provides different types of cells for the production of a Neu(n)Ac-containing compound with a metabolically engineered cell.

According to the method and/or cell of the invention, the cell expresses a Neu(n)Ac synthase that synthesizes a Neu(n)Ac molecule as defined herein. In a preferred embodiment of the method and/or cell, the cell expresses more than one Neu(n)Ac synthase that synthesize any one or more Neu(n)Ac molecules as defined herein.

According to one embodiment of the method and/or cell of the present invention, the cell preferably expresses at least one Neu(n)Ac synthase that has Neu(n)Ac synthase activity and that has PFAM domain PF03102 and/or PatricDB global family domain PGF_06907304 and that comprises the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid. Said PFAM domain is classified as defined on Pfam 32.0 as released in Sept 2018. Said PatricDB domain is classified as defined on PATRIC 3.6.9 as released in March 2020.

In another preferred embodiment of the method and/or cell of the present invention, at least one of the Neu(n)Ac synthases expressed in the cell comprises a polypeptide sequence according to any one of SEQ ID NO 02, 03, 04, 05, 06, 07, 08, 09, 10, 11, 12, 13, 14, 15, 16, 17 or 18. In an alternative preferred embodiment of the method and/or cell of the present invention, the cell expresses at least one Neu(n)Ac synthase that is a functional homolog, variant or derivative of any one of SEQ ID NOs 02, 03, 04, 05, 06, 07, 08, 09, 10, 11, 12, 13, 14, 15, 16, 17 or 18 having at least 80% overall sequence identity to the full-length of any one of said polypeptides with SEQ ID NOs 02, 03, 04, 05, 06, 07, 08, 09, 10, 11, 12, 13, 14, 15, 16, 17 or 18 and having Neu(n)Ac synthase activity. In the context of the present invention, it is preferred that said functional homolog, variant or derivative comprises the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid. At least 80 % overall sequence identity to the full length of any one of said polypeptides with SEQ ID NOs 02, 03, 04, 05, 06, 07, 08, 09, 10, 11, 12, 13, 14, 15, 16, 17 or 18 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of the polypeptides with SEQ ID NOs 02, 03, 04, 05, 06, 07, 08, 09, 10, 11, 12, 13, 14, 15, 16, 17 or 18, respectively, as given herein.

In a preferred embodiment of the method and/or cell of present invention, the cell is metabolically engineered to comprise a pathway for the production of a Neu(n)Ac-containing compound as defined herein. In an alternative preferred embodiment of the method and/or cell of present invention, the cell is metabolically engineered to comprise a pathway for the production of a Neu(n)Ac-containing compound and to have modified expression or activity of a Neu(n)Ac synthase of present invention.

In a further preferred embodiment of the method and/or cell of present invention, the cell comprises a recombinant glycosyltransferase capable of modifying lactose or another acceptor as defined herein with one or more Neu(n)Ac molecules that is/are synthesized by any one or more Neu(n)Ac synthases expressed in the cell as presented herein, into a Neu(n)Ac-containing compound.

In a preferred embodiment of the method and/or cell of the invention, the metabolically engineered cell is modified, preferably has been modified, with gene expression modules wherein the expression from any one of said expression modules is constitutive or is tuneable. Throughout the application and claims, the expression “cell is modified” is preferably replaced with “cell has been modified”.

Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred embodiment of the present invention, the cell is modified with one or more expression modules. The expression modules can be integrated in the genome of said cell or can be presented to said cell on a vector. Said vector can be present in the form of a plasmid, cosmid, phage, liposome, or virus, which is to be stably transformed/transfected into said metabolically engineered cell.

Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the synthesis of said Neu(n)Ac-containing compound; or said recombinant gene is linked to other pathways in said host cell that are not involved in the synthesis of said Neu(n)Ac-containing compound. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

According to a preferred aspect of the present invention, the expression of each of said expression modules is constitutive or tuneable as defined herein.

In a further embodiment of the method and/or cell of the invention, the cell is modified in the expression or activity of at least one of said Neu(n)Ac synthases. In a preferred embodiment, said Neu(n)Ac synthase is an endogenous protein of the cell with a modified expression or activity, preferably said endogenous Neu(n)Ac synthase is overexpressed; alternatively said Neu(n)Ac synthase is an heterologous protein that is heterogeneously introduced and expressed in said cell, preferably overexpressed. Said endogenous Neu(n)Ac synthase can have a modified expression in the cell which also expresses a heterologous Neu(n)Ac synthase.

According to a preferred embodiment of the method and/or cell of the invention, the cell comprises a pathway for production of a Neu(n)Ac-containing compound comprising at least one Neu(n)Ac synthase according to present invention. According to another preferred embodiment of the method and/or cell of the invention, said pathway for production of a Neu(n)Ac-containing compound further comprises at least one enzyme chosen from the list comprising N-acylglucosamine 2-epimerase, UDP-N-acetylglucosamine 2-epimerase, N-acetylmannosamine-6-phosphate 2-epimerase, bifunctional UDP-GIcNAc 2-epimerase/kinase, N-acylneuraminate-9-phosphate synthetase, phosphatase, CMP-sialic acid synthase and sialyltransferase.

In a preferred embodiment, the cell comprises a pathway for production of a Neu(n)Ac-containing compound wherein said cell expresses at least one enzyme chosen from the list comprising an N-acylglucosamine 2-epimerase like is known e.g. from several species including Bacteroides ovatus, E. coli, Homo sapiens, Rattus norvegicus, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. N-acyl-D-glucosamine (GIcNAc) can be added to the cell and/or can be provided by an enzyme expressed in the cell or by the mechanism of the cell. Such cell producing GIcNAc can express a phosphatase converting GIcNAc-6-phosphate into GIcNAc, like any one or more of e.g. the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO18122225. Preferably, the cell is modified to produce GIcNAc. More preferably, the cell is modified for enhanced GIcNAc production. Said modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a Neu(n)Ac-containing compound wherein said cell expresses at least one enzyme chosen from the list comprising an UDP-N-acetylglucosamine 2-epimerase like is known e.g. from several species including Campylobacter jejuni, E. coli, Neisseria meningitidis, Bacillus subtilis, Citrobacter rodentium, a Neu(n)Ac synthase as is disclosed in present invention, a CMP sialic acid synthase like is known e.g from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine (UDP-GIcNAc) can be added to the cell and/or can be provided by an enzyme expressed in the cell or by the metabolism of the cell. Such cell producing an UDP-GIcNAc can express enzymes converting, e.g. GIcNAc, which is to be added to the cell, to UDP-GIcNAc. These enzymes may be any one or more enzymes chosen from the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GIcNAc. More preferably, the cell is modified for enhanced UDP-GIcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

In an alternative and/or additional preferred embodiment, the cell comprises a pathway for production of a Neu(n)Ac-containing compound wherein said cell expresses at least one enzyme chosen from the list comprising an N-acetylmannosamine-6-phosphate 2-epimerase like is known e.g. from several species including E. coli, Haemophilus influenzae, Enterobacter sp., Streptomyces sp., an N-acylneuraminate-9-phosphate synthetase, an N-acylneuraminate-9-phosphate phosphatase like is known e.g. from Candidatus Magnetomorum sp. HK-1 or Bacteroides thetaiotaomicron, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. N-acetyl-D-glucosamine 6-phosphate (GIcNAc-6P) can be added to the cell and/or can be provided by an enzyme expressed in the cell or by the metabolism of the cell. Such cell producing GIcNAc-6P can express an enzyme converting, e.g., GIcN6P, which is to be added to the cell, to GIcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GIcNAc-6P. More preferably, the cell is modified for enhanced GIcNAc-6P production. Said modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a Neu(n)Ac-containing compound wherein said cell expresses at least one enzyme chosen from the list comprising a bifunctional UDP-GIcNAc 2-epimerase/kinase like is known e.g. from several species including Homo sapiens, Rattus norvegicus and Mus musculus, an N-acylneuraminate-9-phosphate synthetase, an N-acylneuraminate-9-phosphate phosphatase like is known e.g. from Candidatus Magnetomorum sp. HK-1 or Bacteroides thetaiotaomicron, a Neu(n)Ac synthase as is disclosed in present invention, a CMP sialic acid synthase like is known e.g. from Neisseria meningitidis, and a sialyltransferase including an alpha-2,3-sialyltransferase, an alpha-2,6-sialyltransferase and/or an alpha-2,8-sialyltransferase, wherein the enzymes are as defined herein. UDP-N-acetylglucosamine can be added to the cell and/or can be provided by an enzyme expressed in the cell or by the metabolism of the cell. Such cell producing an UDP-GIcNAc can express enzymes converting, e.g. GIcNAc, which is to be added to the cell, to UDP-GIcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GIcNAc. More preferably, the cell is modified for enhanced UDP-GIcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the host cell used herein is optionally genetically modified to import an acceptor in the cell, by the introduction and/or overexpression of a transporter able to import the respective acceptor in the cell. Such transporter is for example a membrane protein belonging to the major facilitator superfamily (MFS), the ATP-binding cassette (ABC) transporter family or the PTS system involved in the uptake of e.g. mono-, di- and/or oligosaccharides.

Additionally, or alternatively, the host cell used herein is optionally genetically modified to produce polyisoprenoid alcohols like e.g. phosphorylated dolichol that can act as lipid carrier.

Additionally, or alternatively, the host cell used herein is optionally genetically modified to import lactose in the cell, by the introduction and/or overexpression of a lactose permease. Said lactose permease is for example encoded by the lacY gene or the lac12 gene.

Additionally, or alternatively, the host cell expresses a membrane protein that is a transporter protein involved in transport of compounds and/or the bioproduct as defined in present invention out of the cell, preferably across the outer membrane of the cell wall. Preferably the cell is transformed to comprise at least one nucleic acid sequence encoding a protein selected from the group comprising a lactose transporter like e.g. the LacY or lac12 permease, a glucose transporter, a galactose transporter, a transporter for a nucleotide-activated sugar like for example a transporter for UDP-GIcNAc, a transporter protein involved in transport of a bioproduct, like e.g. Neu(n)Ac-containing compound out of the cell, preferably across the outer membrane of the cell wall. Preferably the cell is transformed to comprise at least one nucleic acid sequence encoding a membrane transporter protein selected from the group comprising a siderophore exporter, a major facilitator superfamily (MFS) transporter, an ATP-binding cassette (ABC) transporter or a sugar efflux transporter.

According to another embodiment of the method and/or cell of the present invention, the cell is capable to express a N-acylneuraminate-9-phosphate synthetase that has N-acylneuraminate-9-phosphate synthetase activity and that has PFAM domain PF03102 and/or PatricDB global family domain PGF_06907304 and that comprises the sequence [DE]XGXNHXGXXXXXXXMXXX[ACPS]XXXXXXXX[KR](Xa)[KR](Xb)[DE](Xc)KXXS with SEQ ID NO 19, wherein X is any amino acid, a is 28 to 32, b is 28 to 30 and c is 14 to 16. Said PFAM domain is classified as defined on Pfam 32.0 as released in Sept 2018. Said PatricDB domain is classified as defined on PATRIC 3.6.9 as released in March 2020. In a preferred embodiment of the method and/or cell of the present invention, the N-acylneuraminate-9-phosphate synthetase has N-acylneuraminate-9-phosphate synthetase activity, has PFAM domain PF03102 and/or PatricDB global family domain PGF_06907304 and comprises the sequence [DE]XGXNHXGXXXXXXXMXX(X, no I, L, M)[AS]XXXXXXXX[KR](Xa)[KR](Xb)[DE](Xc)KXXS with SEQ ID NO 20, wherein X is any amino acid, a is 28 to 32, b is 28 to 30 and c is 14 to 16.

In another preferred embodiment of the method and/or cell of the present invention, the N-acylneuraminate-9-phosphate synthetase expressed in the cell comprises a polypeptide sequence according to any one of SEQ ID NO 21, 22, 23, 24, 25, 26, 27 or 28. In an alternative preferred embodiment of the method and/or cell of the present invention, the cell expresses a N-acylneuraminate-9-phosphate synthetase that is a functional homolog, variant or derivative of any one of SEQ ID NOs 21, 22, 23, 24, 25, 26, 27 or 28 having at least 80% overall sequence identity to the full-length of any one of said polypeptides with SEQ ID NOs 21, 22, 23, 24, 25, 26, 27 or 28 and having N-acylneuraminate-9-phosphate synthetase activity. In the context of the present invention, it is preferred that said functional homolog, variant or derivative comprises the sequence [DE]XGXNHXGXXXXXXXMXXX[ACPS]XXXXXXXX[KR](Xa)[KR](Xb)[DE](Xc)KXXS with SEQ ID NO 19, wherein X is any amino acid, a is 28 to 32, b is 28 to 30 and c is 14 to 16, preferably comprises the sequence [DE]XGXNHXGXXXXXXXMXX(X, no I, L, M)[AS]XXXXXXXX[KR](Xa)[KR](Xb)[DE](Xc)KXXS with SEQ ID NO 20, wherein X is any amino acid, a is 28 to 32, b is 28 to 30 and c is 14 to 16. At least 80% overall sequence identity to the full length of any one of said polypeptides with SEQ ID NOs 21, 22, 23, 24, 25, 26, 27 or 28 should be understood as at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to any one of the polypeptides with SEQ ID NOs 21, 22, 23, 24, 25, 26, 27 or 28, respectively, as given herein.

In a further embodiment of the method and/or cell of the invention, the cell is modified in the expression or activity of at least one of said N-acylneuraminate-9-phosphate synthetases. In a preferred embodiment, said N-acylneuraminate-9-phosphate synthetase is an endogenous protein of the cell with a modified expression or activity, preferably said endogenous N-acylneuraminate-9-phosphate synthetase is overexpressed; alternatively said N-acylneuraminate-9-phosphate synthetase is an heterologous protein that is heterogeneously introduced and expressed in said cell, preferably overexpressed. Said endogenous N-acylneuraminate-9-phosphate synthetase can have a modified expression in the cell which also expresses a heterologous N-acylneuraminate-9-phosphate synthetase.

According to another embodiment of the method and/or cell of the present invention, the cell is capable to express a phosphatase that is a HAD-like phosphatase that has N-acylneuraminate-9-phosphatase activity and that comprises the sequence DXDGXXTDXXXXXXXXGXXXXXXXXXDXXXXXXXXXXXXXXX[ILV]X[ST]XXXXXXXXXRXXXL(Xa)K(Xb)GXDXXD(Xc) GXGXXR[DE] with SEQ ID NO 29, wherein X is any amino acid, a is 10 to 11, b is 21 and c is 32. In an exemplary embodiment, the cell expresses a HAD-like phosphatase with SEQ ID NO 30 that has N-acylneuraminate-9-phosphatase activity.

According to an alternative embodiment of the method and/or cell of the present invention, the cell is capable to express a phosphatase that is a HAD-like phosphatase that has N-acylneuraminate-9-phosphatase activity and that comprises the sequence DXDXT[IL](Xa)TNGXXXXQXXK[IL](Xb)[KR]PXXX[IL][FWY](Xc)G[DN]XXXXD[ILV]XG with SEQ ID NO 31, wherein X is any amino acid, a is 110 to 160, b is 20 to 23 and c is 17. In an exemplary embodiment, the cell expresses a HAD-like phosphatase with SEQ ID NO 32 that has N-acylneuraminate-9-phosphatase activity.

In a further embodiment of the method and/or cell of the invention, the cell is modified in the expression or activity of at least one of said N-acylneuraminate-9-phosphatases. In a preferred embodiment, said N-acylneuraminate-9-phosphatase is an endogenous protein of the cell with a modified expression or activity, preferably said endogenous N-acylneuraminate-9-phosphatase is overexpressed; alternatively said N-acylneuraminate-9-phosphatase is an heterologous protein that is heterogeneously introduced and expressed in said cell, preferably overexpressed. Said endogenous N-acylneuraminate-9-phosphatase can have a modified expression in the cell which also expresses a heterologous N-acylneuraminate-9-phosphatase.

According to another preferred embodiment of the method and/or cell of the present invention, the cell is capable to synthesize N-acetylmannosamine (ManNAc), N-acetylmannosamine-6-phosphate (ManNAc-6-phosphate) and/or phosphoenolpyruvate (PEP).

In a preferred embodiment, the cell comprises a pathway for production of a bioproduct of present invention comprising a pathway for production of ManNAc. ManNAc can be provided by an enzyme expressed in the cell or by the mechanism of the cell. Such cell producing ManNAc can express an N-acylglucosamine 2-epimerase like is known e.g. from several species including Bacteroides ovatus, E. coli, Homo sapiens, Rattus norvegicus that converts GIcNAc into ManNAc. Alternatively, and/or additionally, the cell producing ManNAc can express an UDP-N-acetylglucosamine 2-epimerase like is known e.g. from several species including Campylobacter jejuni, E. coli, Neisseria meningitidis, Bacillus subtilis, Citrobacter rodentium that converts UDP-GIcNAc into ManNAc. GIcNAc and/or UDP-GIcNAc can be added to the cell and/or provided by an enzyme expressed in the cell or by the mechanism of the cell as described herein.

In a more preferred embodiment, the cell is modified for enhanced ManNAc production. Said modification can be any one or more chosen from the group comprising knock-out of N-acetylmannosamine kinase, over-expression of N-acetylneuraminate lyase.

In another preferred embodiment, the cell comprises a pathway for production of a bioproduct of present invention comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GIcNAc 2-epimerase/kinase like is known e.g. from several species including Homo sapiens, Rattus norvegicus and Mus musculus that converts UDP-GIcNAc into ManNAc-6-phosphate. Alternatively, and/or additionally, the cell producing ManNAc-6-phosphate can express an N-acetylmannosamine-6-phosphate 2-epimerase that converts GIcNAc-6-phosphate into ManNAc-6-phosphate. UDP-GIcNAc and/or GIcNAc-6-phosphate can be added to the cell and/or provided by an enzyme expressed in the cell or by the mechanism of the cell as described herein. In a more preferred embodiment, the cell is modified for enhanced ManNAc-6-phosphate production. Said modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

In another preferred embodiment, the cell comprises a pathway for production of a bioproduct of present invention comprising a pathway for production of phosphoenolpyruvate (PEP).

In another preferred embodiment, the cell comprises a pathway for production of a bioproduct of present invention comprising any one or more modifications for enhanced production and/or supply of PEP.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is for instance encoded by the nagE gene (or the cluster nagABCD) in E. coli or Bacillus species, 2) ManXYZ which encodes the Enzyme II Man complex (mannose PTS permease, protein-Npi- phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance encoded by PtsG/Crr) which takes up glucose and forms glucose-6-phosphate in the cytoplasm, 4) the sucrose-specific PTS transporter which takes up sucrose and forms sucrose-6-phosphate in the cytoplasm, 5) the fructose-specific PTS transporter (for instance encoded by the genes fruA and fruB and the kinase fruK which takes up fructose and forms in a first step fructose-1-phosphate and in a second step fructosel,6 bisphosphate, 6) the lactose PTS transporter (for instance encoded by lacE in Lactococcus casei) which takes up lactose and forms lactose-6-phosphate, 7) the galactitol-specific PTS enzyme which takes up galactitol and/or sorbitol and forms galactitol-1-phosphate or sorbitol-6-phosphate respectively, 8) the mannitol-specific PTS enzyme which takes up mannitol and/or sorbitol and forms mannitol-1-phosphate or sorbitol-6-phosphate respectively, and 9) the trehalose-specific PTS enzyme which takes up trehalose and forms trehalose-6-phosphate.

In another and/or additional preferred embodiment and as a means for enhanced production and/or supply of PEP, the full PTS system is disrupted by disrupting the PtslH/Crr gene cluster. Ptsl (Enzyme I) is a cytoplasmic protein that serves as the gateway for the phosphoenolpyruvate:sugar phosphotransferase system (PTS^{sugar}) of E. coli K-12. Ptsl is one of two (Ptsl and PtsH) sugar non-specific protein constituents of the PTS^{sugar} which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar} It accepts a phosphoryl group from phosphorylated Enzyme I (Ptsl-P) and then transfers it to the EIIA domain of any one of the many sugar-specific enzymes (collectively known as Enzymes II) of the PTS^{sugar}. Crr or EIIA^{G1c} is phosphorylated by PEP in a reaction requiring PtsH and Ptsl.

In another and/or additional preferred embodiment, the cell is further modified to compensate for the deletion of a PTS system of a carbon source by the introduction and/or overexpression of the corresponding permease. These are e.g. permeases or ABC transporters that comprise but are not limited to transporters that specifically import lactose such as e.g. the transporter encoded by the LacYgene from E. coli, sucrose such as e.g. the transporter encoded by the cscB gene from E. coli, glucose such as e.g. the transporter encoded by the ga/P gene from E. coli, fructose such as e.g. the transporter encoded by the frul gene from Streptococcus mutans, or the Sorbitol/mannitol ABC transporter such as the transporter encoded by the cluster SmoEFGKof Rhodobactersphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g. ptsG gene, combined with the introduction and/or overexpression of a glucose permease (e.g. galP of glcP), 2) the deletion of the fructose PTS system, e.g. one or more of the fruB, fruA, fruK genes, combined with the introduction and/or overexpression of fructose permease, e.g. frul, 3) the deletion of the lactose PTS system, combined with the introduction and/or overexpression of lactose permease, e.g. LacY, and/or 4) the deletion of the sucrose PTS system, combined with the introduction and/or overexpression of a sucrose permease, e.g. cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g. ptsG gene, combined with the introduction and/or overexpression of a glucose permease (e.g. galP of glcP), combined with the introduction and/or overexpression of a glucokinase (e.g. glk), and/or 2) the deletion of the fructose PTS system, e.g. one or more of thefruB, fruA, fruK genes, combined with the introduction and/or overexpression of fructose permease, e.g. frul, combined with the introduction and/or overexpression of a fructokinase (e.g. frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded for instance in E. coli by ppsA), phosphoenolpyruvate carboxykinase activity (EC 4.1.1.32 or EC 4.1.1.49 encoded for instance in Corynebacterium glutamicum by PCK or in E. coli by pckA, resp.), phosphoenolpyruvate carboxylase activity (EC 4.1.1.31 encoded for instance in E. coli by ppc), oxaloacetate decarboxylase activity (EC 4.1.1.112 encoded for instance in E. coli by eda), pyruvate kinase activity (EC 2.7.1.40 encoded for instance in E. coli by pykA and pykF), pyruvate carboxylase activity (EC 6.4.1.1 encoded for instance in B. subtilis by pyc) and malate dehydrogenase activity (EC 1.1.1.38 or EC 1.1.1.40 encoded for instance in E. coli by maeA or maeB, resp.).

In a more preferred embodiment, the cell is modified to overexpress any one or more of the polypeptides comprising ppsA from E. coli with SEQ ID NO 73, PCK from C. glutamicum with SEQ ID NO 74, pcka from E. coliwith SEQ ID NO 75, eda from E. coliwith SEQ ID NO 76, maeA from E. coliwith SEQ ID NO 77 and maeB from E. coli with SEQ ID NO 78.

In another and/or additional preferred embodiment, the cell is modified to express any one or more of a functional homolog, variant or derivative of any one of SEQ ID NO 73, 74, 75, 76, 77 or 78 having at least 80% overall sequence identity to the full-length of any one of said polypeptide with SEQ ID NOs 73, 74, 75, 76, 77 or 78, and having phosphoenolpyruvate synthase activity, phosphoenolpyruvate carboxykinase activity, oxaloacetate decarboxylase activity, or malate dehydrogenase activity, respectively.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically modified by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically modified by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene.

According to another embodiment of the method and/or cell of the invention, the cell is further capable to synthesize any one or more nucleotide-activated sugars. In a preferred embodiment of the method and/or cell of the invention, the cell is capable to synthesize one or more nucleotide-activated sugars chosen from the list comprising UDP-N-acetylglucosamine (UDP-GIcNAc), UDP-N-acetylgalactosamine (UDP-GaINAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose. In a more preferred embodiment of the method and/or cell of the invention, the cell is capable to synthesize at least nucleotide-activated sugar that is derived from Neu(n)Ac comprising CMP-Neu4Ac, CMP-Neu5Ac, CMP-Neu5Ac9N₃, CMP-Neu4,5Ac₂, CMP-Neu5,7Ac₂, CMP-Neu5,9Ac₂, CMP-Neu5,7(8,9)Ac₂ and CMP-Neu5Gc. In an even more preferred embodiment of the method and/or cell of the invention, the cell uses at least one of the synthesized nucleotide-activated sugars in the production of a bioproduct of present invention like e.g. a Neu(n)Ac-containing compound.

The host cell used herein is optionally genetically modified to express the de novo synthesis of UDP-GIcNAc. UDP-GIcNAc can be provided by an enzyme expressed in the cell or by the metabolism of the cell.

Such cell producing an UDP-GIcNAc can express enzymes converting, e.g. GIcNAc, which is to be added to the cell, to UDP-GIcNAc. These enzymes may be any one or more of the list comprising an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GIcNAc. More preferably, the cell is modified for enhanced UDP-GIcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the host cell used herein is optionally genetically modified to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of an glucosamine-6-phosphate deaminase, over-expression of a CMP-sialic acid synthetase, and over-expression of an N-acetyl-D-glucosamine-2-epimerase encoding gene.

Additionally, or alternatively, the host cell used herein is optionally genetically modified to express the de novo synthesis of CMP-Neu5Gc. CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production.

Additionally, or alternatively, the host cell used herein is optionally genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroidesfragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the host cell used herein is optionally genetically modified to express the de novo synthesis of UDP-Gal. UDP-Gal can be provided by an enzyme expressed in the cell or by the metabolism of the cell. Such cell producing UDP-Gal can express an enzyme converting, e.g. UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of an bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

Additionally, or alternatively, the host cell used herein is optionally genetically modified to express the de novo synthesis of UDP-GaINAc. UDP-GaINAc can be synthesized from UDP-GIcNAc by the action of a single-step reaction using an UDP-N-acetylglucosamine 4-epimerase like e.g. wbgU from Plesiomonas shigelloides, gne from Yersinia enterocolitica or wbpP from Pseudomonas aeruginosa serotype 06.

Preferably, the cell is modified to produce UDP-GaINAc. More preferably, the cell is modified for enhanced UDP-GaINAc production.

Additionally, or alternatively, the host cell used herein is optionally genetically modified to express the de novo synthesis of UDP-ManNAc. UDP-ManNAc can be synthesized directly from UDP-GIcNAc via an epimerization reaction performed by an UDP-GIcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another embodiment of the method and/or cell of the invention, the cell expresses at least one glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the invention, the fucosyltransferase is chosen from the list comprising alpha-1,2-fucosyltransferase, alpha-1,3-fucosyltransferase, alpha-1,4-fucosyltransferase and alpha-1,6-fucosyltransferase.

In an alternative and/or additional embodiment of the method and/or cell of the invention, the sialyltransferase is chosen from the list comprising alpha-2,3-sialyltransferase, alpha-2,6-sialyltransferase, and alpha-2,8-sialyltransferase.

In an alternative and/or additional embodiment of the method and/or cell of the invention, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional embodiment of the method and/or cell of the invention, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the invention, the mannosyltransferase is chosen from the list comprising alpha-1,2-mannosyltransferase, alpha-1,3-mannosyltransferase and alpha-1,6-mannosyltransferase.

In an alternative and/or additional embodiment of the method and/or cell of the invention, the N-acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the method and/or cell of the invention, the N-acetylgalactosaminyltransferase is chosen from the list comprising alpha-1,3-N-acetylgalactosaminyl-transferase.

In a further embodiment of the method and/or cell of the invention, the cell is modified in the expression or activity of at least one of said glycosyltransferases. In a preferred embodiment, said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another preferred embodiment of the method and/or cell of the invention, the cell expresses at least one alpha-2,3-sialyltransferase which has alpha-2,3-sialyltransferase activity.

In a preferred embodiment of the method and/or cell of the invention, the alpha-2,3-sialyltransferase is the polypeptide from Pasteurella multocida with SEQ ID NO 80.

In an alternative and/or additional preferred embodiment of the method and/or cell of the invention, the alpha-2,3-sialyltransferase is a polypeptide of 268 amino acid residues long that is a functional homolog, variant or derivative of said polypeptide with SEQ ID NO 80 having at least 80% overall sequence identity to the full-length of said polypeptide with SEQ ID NO 80 and having alpha-2,3-sialyltransferase activity. A polypeptide of 268 amino acid residues long and having at least 80% overall sequence identity to the full-length of said polypeptide with SEQ ID NO 80 should be understood as a polypeptide of 268 amino acid residues long that has at least 80%, 81%, 82%, 83%, 84%, 85%, 86%, 87%, 88%, 89%, 90%, 91%, 92 %, 93%, 94%, 95%, 96%, 97%, 98%, or 99% overall sequence identity to said polypeptide with SEQ ID 80 as given herein.

In an alternative and/or additional preferred embodiment of the method and/or cell of the invention, the alpha-2,3-sialyltransferase is a polypeptide of 268 amino acid residues long that has one or more single point mutations compared to the polypeptide with SEQ ID NO 80 and having alpha-2,3-sialyltransferase activity.

In another embodiment of the method and/or cell of the invention, the alpha-2,3-sialyltransferase is involved in the production of a bioproduct of present invention comprising a disaccharide, an oligosaccharide, a glycolipid, and/or a glycoprotein. In a preferred embodiment of the method and/or cell of the invention, the alpha-2,3-sialyltransferase is involved in the production of a Neu(n)Ac-containing compound, wherein (n) is 4, 5, 7, 8 or 9 or a combination thereof.

In a further embodiment of the method and/or cell of the invention, the cell is modified in the expression or activity of at least one of said alpha-2,3-sialyltransferases. In a preferred embodiment, said alpha-2,3-sialyltransferase is an endogenous protein of the cell with a modified expression or activity, preferably said endogenous alpha-2,3-sialyltransferase is overexpressed; alternatively said alpha-2,3-sialyltransferase is a heterologous protein that is heterogeneously introduced and expressed in said cell, preferably overexpressed. Said endogenous alpha-2,3-sialyltransferase can have a modified expression in the cell which also expresses a heterologous alpha-2,3-sialyltransferase.

According to another preferred method of the method and/or cell of the invention, the Neu(n)Ac synthase is an Neu5Ac synthase and the Neu(n)Ac-containing compound is a sialylated compound comprising Neu5Ac.

According to another and/or alternative preferred embodiment of the method and/or cell of the invention, the cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase.

According to another and/or alternative preferred embodiment of the method and/or cell of the invention, the cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase.

According to another and/or alternative preferred embodiment of the method and/or cell of the invention, the cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.

According to another preferred embodiment of the method and/or cell of the invention, the cell comprises a modification for reduced production of acetate. Said modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further embodiment of the method and/or cell of the invention, the cell is modified in the expression or activity of at least one acetyl-coenzyme A synthetase like e.g. acs from E. coli, S. cerevisiae, H. sapiens, M. musculus. In a preferred embodiment, said acetyl-coenzyme A synthetase is an endogenous protein of the cell with a modified expression or activity, preferably said endogenous acetyl-coenzyme A synthetase is overexpressed; alternatively, said acetyl-coenzyme A synthetase is a heterologous protein that is heterogeneously introduced and expressed in said cell, preferably overexpressed. Said endogenous acetyl-coenzyme A synthetase can have a modified expression in the cell which also expresses a heterologous can have a modified expression in the cell which also expresses a heterologous. In a more preferred embodiment, the cell is modified in the expression or activity of the acetyl-coenzyme A synthetase acs from E. coli with SEQ ID NO 79. In another and/or additional preferred embodiment, the cell is modified in the expression or activity of a functional homolog, variant or derivative of SEQ ID NO 79 having at least 80% overall sequence identity to the full-length of said polypeptide with SEQ ID NO 79 and having acetyl-coenzyme A synthetase activity.

In an alternative and/or additional further embodiment of the method and/or cell of the invention, the cell is modified in the expression or activity of at least one pyruvate dehydrogenase like e.g. from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further embodiment of the method and/or cell of the invention, the cell is modified in the expression or activity of at least one lactate dehydrogenase like e.g. from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the IdhA encoding gene resulting in a cell lacking lactate dehydrogenase activity.

According to another preferred embodiment of the method and/or cell of the invention, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component malX, enzyme IIA^{G1c}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred embodiment of the method and/or cell of the invention, the cell comprises a catabolic pathway for selected mono-, di- or oligosaccharides which is at least partially inactivated, the mono-, di-, or oligosaccharides being involved in and/or required for the synthesis of a Neu(n)Ac-containing compound.

According to another preferred embodiment of the method and/or cell of the invention, the cell is using a precursor for the synthesis of the bioproduct of present invention comprising a Neu(n)Ac-containing compound. Herein, the precursor is fed to the cell from the cultivation medium. In another preferred embodiment, the cell is producing a precursor for the synthesis of said bioproduct of present invention comprising a Neu(n)Ac-containing compound.

According to another preferred embodiment of the method and/or cell of the invention, the cell produces 90 g/L or more of a bioproduct of present invention like e.g. a Neu(n)Ac-containing compound in the whole broth and/or supernatant. In a more preferred embodiment, bioproduct of present invention like e.g. the Neu(n)Ac-containing compound produced in the whole broth and/or supernatant has a purity of at least 80% measured on the total amount of the bioproduct of present invention like e.g. the Neu(n)Ac-containing compound and its precursor produced by the cell in the whole broth and/or supernatant, respectively.

According to another embodiment of the method and/or cell of the invention, the bioproduct of present invention is chosen from the list comprising monosaccharide, phosphorylated monosaccharide, activated monosaccharide, disaccharide, oligosaccharide, aglycon, glycolipid and/or glycoprotein. In a preferred embodiment, the oligosaccharide is chosen from the list comprising a milk oligosaccharide, 0-antigen, , the oligosaccharide repeats present in capsular polysaccharides, peptidoglycan, amino-sugars and Lewis-type antigen oligosaccharide. In a more preferred embodiment, the milk oligosaccharide is a mammalian milk oligosaccharide (MMO). In an even more preferred embodiment, the milk oligosaccharide is a human milk oligosaccharide (HMO).

According to another embodiment of the method and/or cell of the invention, the Neu(n)Ac-containing compound is chosen from the list comprising sialic acid, a disaccharide, an oligosaccharide, a glycolipid and/or a glycoprotein. In a preferred embodiment of the method and/or cell of the invention, the Neu(n)Ac-containing compound is chosen from the list consisting of sialic acid, a disaccharide, an oligosaccharide, a glycolipid and/or a glycoprotein. In a more preferred embodiment of the method and/or cell of the invention, the Neu(n)Ac-containing compound is chosen from the list consisting of sialic acid, a disaccharide, an oligosaccharide and/or a glycolipid. In an even more preferred embodiment of the method and/or cell of the invention, the Neu(n)Ac-containing compound is chosen from the list consisting of sialic acid, a disaccharide and/or an oligosaccharide. In a preferred embodiment, the oligosaccharide is chosen from the list comprising a milk oligosaccharide, 0-antigen, , the oligosaccharide repeats present in capsular polysaccharides, peptidoglycan, amino-sugars and Lewis-type antigen oligosaccharide. In a more preferred embodiment, the milk oligosaccharide is a mammalian milk oligosaccharide. In an even more preferred embodiment, the milk oligosaccharide is a human milk oligosaccharide.

According to another embodiment of the method and/or cell of the invention, the cell is capable to synthesize a mixture of oligosaccharides. In an alternative and/or additional aspect, the cell is capable to synthesize a mixture of di- and oligosaccharides, alternatively, the cell is capable to synthesize a mixture of sialic acid, di- and/or oligosaccharides.

Another embodiment of the invention provides for a method and a cell wherein a bioproduct of present invention like e.g. a Neu(n)Ac-containing compound is produced in and/or by a bacterial, fungal, yeast, insect, plant, animal or protozoan expression system or cell as described herein. The expression system or cell is chosen from the list comprising a bacterium, a fungus, or a yeast, or, refers to a plant, animal, or protozoan cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus. , such as Bacillus subtilis or, B. amyloliquefaciens. The latter bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter fungus belongs preferably to the genus Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g. Saccharomyces cerevisiae, S. bayanus, S. boulardii), Zygosaccharomyces, Pichia (with members like e.g. Pichia pastoris, P. anomala, P. kluyveri), Komagataella, Hansenula, Yarrowia (like e.g. Yarrowia lipolytica), Starmerella (like e.g. Starmerella bombicola), Kluyveromyces with members like e.g. Kluyveromyces lactis, K. marxianus, K. thermotolerans) or Debaromyces. The latter yeast is preferably selected from Pichia pastoris, Yarrowia lipolitica, Saccharomyces cerevisiae and Kluyveromyces lactis. Plant cells include cells of flowering and non-flowering plants, as well as algal cells, for example Chlamydomonas, Chlorella, etc. Preferably, said plant is a tobacco, alfalfa, rice, tomato, cotton, rapeseed, soy, maize, or corn plant. The latter animal cell is preferably derived from non-human mammals (e.g. cattle, buffalo, pig, sheep, mouse, rat), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a genetically modified cell line derived from human cells excluding embryonic stem cells. Both human and non-human mammalian cells are preferably chosen from the list comprising an epithelial cell like e.g. a mammary epithelial cell, an embryonic kidney cell, a fibroblast cell, a COS cell, a Chinese hamster ovary (CHO) cell, a murine myeloma cell like e.g. an N20, SP2/0 or YB2/0 cell, an NIH-3T3 cell, a non-mammary adult stem cell or derivatives thereof such as described in WO21067641. The latter insect cell is preferably derived from Spodoptera frugiperda like e.g. Sf9 or Sf21 cells, Bombyx mori, Mamestra brassicae, Trichoplusia ni like e.g. BTI-TN-5B1-4 cells or Drosophila melanogaster like e.g. Drosophila S2 cells. The latter protozoan cell preferably is a Leishmania tarentolae cell.

In a preferred embodiment the cell is a cell of a microorganism, wherein more preferably said microorganism is a bacterium or a yeast. In a more preferred embodiment, the microorganism is a bacterium, most preferably Escherichia coli. Examples using such E. coli are described herein.

In another more preferred embodiment, the cell is a yeast.

Another embodiment provides for a cell to be stably cultured in a medium, wherein said medium can be any type of growth medium comprising minimal medium, complex medium or growth medium enriched in certain compounds like for example but not limited to vitamins, trace elements, amino acids.

Throughout the application and claims, unless specified otherwise, the verbs “cultivate” (and its conjugations) and “culture” (and its conjugations) are interchangeably used in the context of the present invention.

The cell as used herein is capable to grow on a monosaccharide, disaccharide, oligosaccharide, polysaccharide, polyol, glycerol, a complex medium or a mixture thereof as the main carbon source. With the term main is meant the most important carbon source for the cell for the production of the bioproduct of interest, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. In one embodiment of the invention, said carbon source is the sole carbon source for said organism, i.e. 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the bioproduct of present invention.

In a further preferred embodiment, the method for the production of a bioproduct of present invention like e.g. a Neu(n)Ac-containing compound as described herein comprises at least one of the following steps:
i) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
ii) Adding at least one precursor and/or acceptor feed in a continuous manner to the culture medium over the course of 1 day, 2 days, 3 days, 4 days, 5 days by means of a feeding solution; iii) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.;
said method resulting in a bioproduct of present invention like e.g. a Neu(n)Ac-containing compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another and/or additional further preferred embodiment, the method for the production of a bioproduct of present invention like e.g. a Neu(n)Ac-containing compound as described herein comprises at least one of the following steps:
i) Adding to the culture medium at least one precursor and/or acceptor in one pulse or in a discontinuous (pulsed) manner wherein the total reactor volume ranges from 250 mL (millilitre) to 10.000 m³ (cubic meter), preferably so that the final volume of the culture medium is not more than three-fold, preferably not more than two-fold, more preferably less than 2-fold of the volume of the culture medium before the addition of said precursor and/or acceptor feed pulse(s);
ii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
iii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.;
said method resulting in a bioproduct of present invention like e.g. a Neu(n)Ac-containing compound with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In a further, more preferred embodiment, the method for the production of a bioproduct of present invention like e.g. a Neu(n)Ac-containing compound as described herein comprises at least one of the following steps:
i) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.; said method resulting in a bioproduct of present invention like e.g. a Neu(n)Ac-modified lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium. Preferably the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

In another aspect the lactose feed is accomplished by adding lactose to the cultivation medium in a concentration, such that throughout the production phase of the cultivation a lactose concentration of at least 5 mM, preferably 10 mM or 30 mM is obtained.

In a further embodiment of the methods described herein the host cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the culture medium in a second phase.

In an alternative preferable embodiment, in the method as described herein, the lactose is added already in the first phase of exponential growth together with the carbon-based substrate.

According to the present invention, the methods as described herein preferably comprises a step of separating the bioproduct of present invention from said cultivation.

The terms “separating from said cultivation” means harvesting, collecting, or retrieving said bioproduct of present invention like e.g. a Neu(n)Ac-containing compound from the cell and/or the medium of its growth.

The bioproduct can be separated in a conventional manner from the aqueous culture medium, in which the cell was grown. In case said bioproduct is still present in the cells producing the bioproduct, conventional manners to free or to extract said bioproduct out of the cells can be used, such as cell destruction using high pH, heat shock, sonication, French press, homogenization, enzymatic hydrolysis, chemical hydrolysis, solvent hydrolysis, detergent, hydrolysis, . . . The culture medium and/or cell extract together and separately can then be further used for separating said bioproduct.

This preferably involves clarifying said bioproduct to remove suspended particulates and contaminants, particularly cells, cell components, insoluble metabolites and debris produced by culturing the genetically modified cell. In this step, said bioproduct can be clarified in a conventional manner. Preferably, said bioproduct is clarified by centrifugation, flocculation, decantation and/or filtration. Another step, preferably a second step, of separating said Neu(n)Ac-containing compound preferably involves removing substantially all the eventually remaining proteins, peptides, amino acids, RNA, DNA, endotoxins and glycolipids that could interfere with the subsequent separation step, from said Neu(n)Ac-containing compound, preferably after it has been clarified. In this step, remaining proteins and related impurities can be removed from said Neu(n)Ac-containing compound in a conventional manner. Preferably, remaining proteins, salts, by-products, colour, endotoxins and other related impurities are removed from said Neu(n)Ac-containing compound by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the bioproduct of present invention. A further purification of said bioproduct may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of said bioproduct. Another purification step is to dry, e.g. spray dry or lyophilize the produced bioproduct.

In an exemplary embodiment, the separation and purification of the bioproduct like e.g. a Neu(n)Ac-containing compound is made in a process, comprising the following steps in any order:
a) contacting the cultivation or a clarified version thereof with a nanofiltration membrane with a molecular weight cut-off (MWCO) of 600-3500 Da ensuring the retention of the produced Neu(n)Ac-containing compound and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said bioproduct in the form of a salt from the cation of said electrolyte.

In an alternative exemplary embodiment, the separation and purification of said bioproduct is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said bioproduct is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of said bioproduct is made in the following way. The cultivation comprising the produced bioproduct, biomass, medium components and contaminants, and wherein the purity of the produced bioproduct like e.g. Neu(n)Ac-containing compound in the cultivation is <80 percent, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step, wherein a purified solution comprising the produced bioproduct at a purity of greater than or equal to 80 percent is provided. Optionally the purified solution is spray dried.

In an alternative exemplary embodiment, the separation and purification of the bioproduct is made in a process, comprising the following steps in any order: enzymatic treatment of the cultivation; removal of the biomass from the cultivation; ultrafiltration; nanofiltration; and a column chromatography step.

Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, I11 and IV with different flow rates; and/or iii) an eluent comprising water; and/or iv) an operating temperature of 15 degrees to 60 degrees centigrade.

In a specific embodiment, the present invention provides the produced bioproduct which is spray-dried to powder, wherein the spray-dried powder contains <15 percent-wt. of water, preferably <10 percent -wt. of water, more preferably <7 percent-wt. of water, most preferably <5 percent-wt. of water.

Another aspect of the present invention provides the use of a cell as defined herein, in a method for the production of a bioproduct like e.g. a Neu(n)Ac-containing compound. A further aspect of the present invention provides the use of a method as defined herein for the production of a bioproduct like e.g. a Neu(n)Ac-containing compound.

Furthermore, the invention also relates to the bioproduct like e.g. the Neu(n)Ac-containing compound obtained by the methods according to the invention, as well as to the use of a polynucleotide, the vector, host cells or the polypeptide as described above for the production of said bioproduct. Said bioproduct may be used as food additive, prebiotic, symbiotic, for the supplementation of baby food, adult food or feed, or as either therapeutically or pharmaceutically active compound or in cosmetic applications. With the novel methods, the bioproduct can easily and effectively be provided, without the need for complicated, time and cost consuming synthetic processes.

For identification of the bioproduct of present invention produced in the cell as described herein, the monosaccharide or the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the bioproduct methods such as e.g. acid-catalysed hydrolysis, HPLC (high performance liquid chromatography) or GLC (gas-liquid chromatography) (after conversion to alditol acetates) may be used. To determine the glycosidic linkages, the bioproduct is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the bioproduct is subjected to enzymatic analysis, e.g. it is contacted with an enzyme that is specific for a particular type of linkage, e.g., beta-galactosidase, or alpha-glucosidase, etc., and NMR may be used to analyse the products.

The separated and preferably also purified bioproduct like e.g. a Neu(n)Ac-containing compound as described herein is incorporated into a food (e.g., human food or feed), dietary supplement, pharmaceutical ingredient, cosmetic ingredient or medicine. In some embodiments, the bioproduct is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the bioproduct being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a bioproduct produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, the bioproduct like e.g. a Neu(n)Ac-containing oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a bioproduct like e.g. a Neu(n)Ac-containing oligosaccharide produced and/or purified by a process in this specification is included in infant formula to provide nutritional benefits similar to those provided by the oligosaccharides in human breast milk. In some embodiments, the bioproduct like a Neu(n)Ac-containing oligosaccharide is mixed with one or more ingredients of the infant formula.

Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N- fucopentaose Ill, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto- N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the bioproduct like a Neu(n)Ac-containing oligosaccharide in the infant formula is approximately the same concentration as the concentration of the bioproduct generally present in human breast milk.

In some embodiments, the bioproduct is incorporated into a feed preparation, wherein said feed is chosen from the list comprising pet food, animal milk replacer, veterinary product, post weaning feed, or creep feed.

As will be shown in the examples herein, the newly identified Neu(n)Ac synthases have proven to be useful in the production, preferably fermentative production, of bioproducts like e.g. Neu(n)Ac-containing compounds. The method and the cell of the invention preferably provide at least one of the following further surprising advantages when using the Neu(n)Ac synthases as defined herein:
High, preferably higher, titres of the Neu(n)Ac-containing compound (g/L),
High, preferably higher, production rate r (g Neu(n)Ac-containing compound/L/h),
High, preferably higher, cell performance index CPI (g Neu(n)Ac-containing compound/g X),
High, preferably higher, specific productivity Qp (g Neu(n)Ac-containing compound/g X/h),
High, preferably higher, yield on sucrose Ys (g Neu(n)Ac-containing compound/g sucrose),
High, preferably higher, sucrose uptake/conversion rate Qs (g sucrose/g X/h),
High, preferably higher, lactose conversion/consumption rate rs (g lactose/h),
High, preferably higher, secretion of the Neu(n)Ac-containing compound, and/or
High, preferably higher, growth speed of the production host,

when compared to a production host for a Neu(n)Ac-containing compound with an identical genetic background but lacking the expression of the homologous and/or heterologous Neu(n)Ac synthase and/or (over)expression of the endogenous Neu(n)Ac synthase. In the present context, “X” means biomass, “g” means gram, “L” means liter and “h” means hour. Said “g Neu(n)Ac-containing compound” can be measured in the whole broth and/or in the supernatans.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis.

Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:
1. A metabolically engineered cell for production of a bioproduct of the list comprising monosaccharide, activated monosaccharide, phosphorylated monosaccharide, disaccharide, oligosaccharide, aglycon, glycolipid or glycoprotein, said cell comprising a pathway for said bioproduct, preferably said pathway comprises at least one glycosyltransferase that is involved in the production of said bioproduct.
2. Cell according to embodiment 1, wherein said bioproduct is an N-acetylneuraminic acid (Neu(n)Ac)-containing compound wherein (n) is 4, 5, 7, 8 or 9 or a combination thereof and wherein said cell is modified in the expression or activity of at least one Neu(n)Ac synthase which has Neu(n)Ac synthase activity and which has PFAM domain PF03102 and/or PatricDB global family domain PGF_06907304 and which
   comprises the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid, or
   comprises a polypeptide sequence according to any one of SEQ ID NO 02, 03, 04, 05, 06, 07, 08, 09, 10, 11, 12, 13, 14, 15, 16, 17 or 18, or
   is a functional homolog, variant or derivative of any one of SEQ ID NO 02, 03, 04, 05, 06, 07, 08, 09, 10, 11, 12, 13, 14, 15, 16, 17 or 18 having at least 80% overall sequence identity to the full-length of any one of said polypeptide with SEQ ID NO 02, 03, 04, 05, 06, 07, 08, 09, 10, 11, 12, 13, 14, 15, 16, 17 or 18 and having Neu(n)Ac synthase activity, respectively.
3. Cell according to any one of embodiment 1 or 2, wherein said modification comprises overexpression of an endogenous Neu(n)Ac synthase and/or introduction and expression of a homologous or heterologous Neu(n)Ac synthase.
4. Cell according to any one of embodiment 1 to 3, wherein said Neu(n)Ac synthase is presented to the cell in one or more gene expression modules wherein expression is regulated by one or more regulatory sequences.
5. Cell according to any one of previous embodiments, wherein said expression modules are integrated in the host cell's genome and/or presented to the cell on a vector comprising plasmid, cosmid, phage, liposome or virus, which is to be stably transformed into said host cell.
6. Cell according to any one of previous embodiments, wherein said pathway for production of said Neu(n)Ac-containing compounds comprises at least one enzyme chosen from the list comprising N-acylglucosamine 2-epimerase, UDP-N-acetylglucosamine 2-epimerase, N-acetylmannosamine-6-phosphate 2-epimerase, bifunctional UDP-GlcNAc 2-epimerase/kinase, N-acylneuraminate-9-phosphate synthetase, phosphatase, CMP sialic acid synthase, sialyltransferase.
7. Cell according to embodiment 6, wherein said N-acylneuraminate-9-phosphate synthetase is a polypeptide having N-acylneuraminate-9-phosphate synthetase activity and which has PFAM domain PF03102 and/or PatricDB global family domain PGF_06907304 and which:
   comprises the sequence
   [DE]XGXNHXGXXXXXXXMXXX[ACPS]XXXXXXXX[KR](Xa)[KR](Xb)[DE](Xc)KXXS with SEQ ID NO 19, wherein X is any amino acid, a is 28 to 32, b is 28 to 30 and c is 14 to 16; preferably comprises the sequence [DE]XGXNHXGXXXXXXXMXX(X, no I, L, M)[AS]XXXXXXXX[KR](Xa)[KR](Xb)[DE](Xc)KXXS with SEQ ID NO 20, wherein X is any amino acid, a is 28 to 32, b is 28 to 30 and c is 14 to 16, or
   comprises a polypeptide sequence according to any one of SEQ ID NO 21, 22, 23, 24, 25, 26, 27 or 28, or
   is a functional homolog, variant or derivative of any one of SEQ ID NO 21, 22, 23, 24, 25, 26, 27 or 28 having at least 80% overall sequence identity to the full-length of any one of said polypeptide with SEQ ID NO 21, 22, 23, 24, 25, 26, 27 or 28 and having N-acylneuraminate-9-phosphate synthetase activity, respectively.
8. Cell according to embodiment 6, wherein said phosphatase is a HAD-like phosphatase having N-acylneuraminate-9-phosphatase activity and:
   comprising the sequence
   DXDGXXTDXXXXXXXXGXXXXXXXXXDXXXXXXXXXXXXXXX[ILV]X[ST]XXXXXXXXXRXXXL(Xa)K(Xb)GX DXXD(Xc)GXGXXR[DE] with SEQ ID NO 29, wherein X is any amino acid, a is 10 to 11, b is 21 and c is 32, or
   comprising the sequence
   DXDXT[IL](Xa)TNGXXXXQXXK[IL](Xb)[KR]PXXX[IL][FWY](Xc)G[DN]XXXXD[ILV]XG with SEQ ID NO 31, wherein X is any amino acid, a is 110 to 160, b is 20 to 23 and c is 17.
9. Cell according to any one of previous embodiments, wherein said cell is capable to synthesize N-acetylmannosamine (ManNAc), N-acetylmannosamine-6-phosphate (ManNAc-6-phosphate) and/or phosphoenolpyruvate (PEP).
10. Cell according to any one of previous embodiments, wherein said cell is modified for enhanced synthesis and/or supply of PEP.
11. Cell according to any one of previous embodiments, wherein said cell is further capable to synthesize any one or more nucleotide-activated sugars.
12. Cell according to embodiment 11, wherein said nucleotide-activated sugar is chosen from the list comprising UDP-N-acetylglucosamine (UDP-GIcNAc), UDP-N-acetylgalactosamine (UDP-GaINAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
13. Cell according to any one of embodiment 11 or 12, wherein at least one of said nucleotide-activated sugars is derived from Neu(n)Ac, comprising CMP-Neu4Ac, CMP-Neu5Ac, CMP-Neu5Ac9N₃, CMP-Neu4,5Ac₂, CMP-Neu5,7Ac₂, CMP-Neu5,9Ac₂, CMP-Neu5,7(8,9)Ac₂ and CMP-Neu5Gc.
14. Cell according to any one of embodiment 11 to 13, wherein said cell uses at least one of said nucleotide-activated sugars in the production of said Neu(n)Ac-containing compound.
15. Cell according to any one of previous embodiments, wherein said glycosyltransferase is chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, wherein said cell is modified in the expression or activity of at least one of said glycosyltransferases,
   preferably, said fucosyltransferase is chosen from the list comprising alpha-1,2-fucosyltransferase, alpha-1,3-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase, and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is chosen from the list comprising alpha-1,3-N-acetylgalactosaminyltransferase.
16. Cell according to any one of previous embodiments, wherein said cell expresses at least one alpha-2,3-sialyltransferase which has alpha-2,3-sialyltransferase activity and which
   is the polypeptide from Pasteurella multocida with SEQ ID NO 80, or
   is a polypeptide of 268 amino acid residues long that is a functional homolog, variant or derivative of said polypeptide with SEQ ID NO 80 having at least 80% overall sequence identity to the full-length of said polypeptide with SEQ ID NO 80 and having alpha-2,3-sialyltransferase activity, preferably, said cell is modified in the expression or activity of any one of said alpha-2,3-sialyltransferase.
17. Cell according to any one of previous embodiments, wherein said Neu(n)Ac synthase is an Neu5Ac synthase and wherein the Neu(n)Ac-containing compound is a sialylated compound comprising Neu5Ac.
18. Cell according to any one of previous embodiments, wherein said cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase.
19. Cell according to any one of previous embodiments, wherein said cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase.
20. Cell according to any one of previous embodiments, wherein said cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.
21. Cell according to any one of previous embodiments, wherein said cell comprises a modification for reduced production of acetate.
22. Cell according to any one of previous embodiments, wherein said cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component malX, enzyme IIAG¹c, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.
23. Cell according to any one of previous embodiments, wherein said cell comprises a catabolic pathway for selected mono-, di- or oligosaccharides which is at least partially inactivated, the mono-, di-, or oligosaccharides being involved in and/or required for the synthesis of said Neu(n)Ac-containing compound.
24. Cell according to any one of previous embodiments, wherein said cell is using a precursor for the synthesis of said bioproduct, said precursor being fed to the cell from the cultivation medium.
25. Cell according to any one of previous embodiments, wherein said cell is producing a precursor for the synthesis of said bioproduct.
26. Cell according to any one of previous embodiments, wherein said cell produces 90 g/L or more of said bioproduct in the whole broth and/or supernatant and/or wherein said Neu(n)Ac-containing compound in the whole broth and/or supernatant has a purity of at least 80% measured on the total amount of bioproduct and its precursor produced by said cell in the whole broth and/or supernatant, respectively.
27. Cell according to any one of previous embodiments, wherein said Neu(n)Ac-containing compound is chosen from the list comprising sialic acid, a disaccharide, an oligosaccharide, a glycolipid and/or a glycoprotein.
28. Cell according to any one of previous embodiments, wherein said oligosaccharide is chosen from the list comprising a milk oligosaccharide, 0-antigen, , an oligosaccharide repeat present in capsular polysaccharides, peptidoglycan, amino-sugars and Lewis-type antigen oligosaccharide, preferably said milk oligosaccharide is a mammalian milk oligosaccharide, more preferably said milk oligosaccharide is a human milk oligosaccharide.
29. Cell according to any one of previous embodiments, wherein the cell is capable to synthesize a mixture of oligosaccharides.
30. Cell according to any one of previous embodiments, wherein the cell is capable to synthesize a mixture of di- and oligosaccharides.
31. Cell according to any one of previous embodiments, wherein the cell is capable to synthesize a mixture of sialic acid, a di- and/or oligosaccharides.
32. Cell according to any one of previous embodiments, wherein said cell is a bacterium, fungus, yeast, a plant cell, an animal cell, or a protozoan cell,
   preferably said bacterium is of an Escherichia coli strain, more preferably of an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
preferably said protozoan cell is a Leishmania tarentolae cell.
33. Cell according to any one of previous embodiments, wherein said cell is stably cultured in a medium.
34. Method to produce a bioproduct by a cell, the method comprising the steps of:
   1) providing a cell according to any one embodiment 1 to 33, and
   2) cultivating said cell under conditions permissive to produce said bioproduct,
   3) preferably, separating said bioproduct from said cultivation, preferably, wherein said bioproduct is a Neu(n)Ac-containing compound.
35. Method according to embodiment 34, the method further comprising at least one of the following steps:
   i) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed; ii) Adding at least one precursor and/or acceptor feed in a continuous manner to the culture medium over the course of 1 day, 2 days, 3 days, 4 days, 5 days by means of a feeding solution; iii) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.; said method resulting in a bioproduct, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium, preferably wherein said bioproduct is a Neu(n)Ac-containing compound.
36. Method according to embodiment 34, the method further comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   ii) adding a lactose feed in a continuous manner to the culture medium over the course of 1 day, 2 days, 3 days, 4 days, 5 days by means of a feeding solution;
   iii) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.; said method resulting in a bioproduct produced from said lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium, preferably, wherein said bioproduct produced from said lactose comprises one or more Neu(n)Ac molecule(s).
37. Method according to embodiment 36, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.
38. Method according to any one of embodiment 36 or 37, wherein said lactose feed is accomplished by adding lactose to the cultivation medium in a concentration, such, that throughout the production phase of the cultivation a lactose concentration of at least 5 mM, preferably 10 mM or 30 mM is obtained.
39. Method according to any one of embodiment 34 to 38, wherein the host cells are cultivated for at least about 60, 80, 100, or about 120 hours or in a continuous manner.
40. Method according to any one of embodiment 34 to 39, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
41. Method according to any one of embodiment 34 to 40, wherein said cell uses at least one precursor for the synthesis of said bioproduct, preferably said cell uses two or more precursors for the synthesis of said bioproduct.
42. Method according to any one of embodiment 34 to 41, wherein the culture medium contains at least one compound selected from the group comprising lactose, galactose, sialic acid, fucose, GlcNAc, GaINAc, lacto-N-biose (LNB), N-acetyllactosamine (LacNAc).
43. Method according to any one of embodiment 34 to 42, wherein a first phase of exponential cell growth is provided by adding a carbon-based substrate, preferably glucose or sucrose, to the culture medium before the lactose is added to the culture medium in a second phase.
44. Method according to any one of embodiment 34 to 43, wherein said cell is producing at least one precursor for the synthesis of said bioproduct.
45. Method according to any one of embodiment 34 to 44, wherein said precursor for the synthesis of said bioproduct is completely converted into said bioproduct.
46. Method according to any one of embodiment 34 to 45, wherein the bioproduct is separated from the culture medium and/or the cell.
47. Method according to any one of embodiment 34 to 46, wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography.
48. Method according to any one of embodiment 34 to 47, wherein said method further comprises purification of said bioproduct.
49. Method according to embodiment 48, wherein said purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying or lyophilization.
50. Use of a cell according to any one of embodiment 1 to 33 for production of a bioproduct.
51. Use of a cell according to any one of embodiment 1 to 33 for production of a Neu(n)Ac-containing compound, preferably wherein (n) is 4, 5, 7, 8 or 9 or a combination thereof.
52. Use of a method according to any one of embodiment 34 to 49 for production of a bioproduct.
53. Use of a method according to any one of embodiment 34 to 49 for production of a Neu(n)Ac-containing compound, preferably wherein (n) is 4, 5, 7, 8 or 9 or a combination thereof.

Moreover, the invention relates to the following preferred specific embodiments:
1. A metabolically engineered cell for production of a bioproduct of the list comprising monosaccharide, activated monosaccharide, phosphorylated monosaccharide, disaccharide, oligosaccharide, aglycon, glycolipid or glycoprotein, said cell comprising a pathway for said bioproduct, preferably said pathway comprises at least one glycosyltransferase that is involved in the production of said bioproduct.
   2. Cell according to claim 1, wherein said bioproduct is an N-acetylneuraminic acid (Neu(n)Ac)-containing compound wherein (n) is 4, 5, 7, 8 or 9 or a combination thereof and wherein said cell is modified, preferably has been modified, in the expression or activity of at least one Neu(n)Ac synthase which has Neu(n)Ac synthase activity and which has PFAM domain PF03102 and/or PatricDB global family domain PGF_06907304 and which comprises the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid.
   3. Cell according to claim 1 or 2, wherein said bioproduct is an N-acetylneuraminic acid (Neu(n)Ac)-containing compound wherein (n) is 4, 5, 7, 8 or 9 or a combination thereof and wherein said cell is modified, preferably has been modified, in the expression or activity of at least one Neu(n)Ac synthase which has Neu(n)Ac synthase activity and which has PFAM domain PF03102 and/or PatricDB global family domain PGF_06907304 and which
   comprises or consists of a polypeptide sequence according to any one of SEQ ID NO 02, 03, 04, 05, 06, 07, 08, 09, 10, 11, 12, 13, 14, 15, 16, 17 or 18, or
   is a functional homolog, variant, derivative or functional fragment, preferably is a functional homolog or functional fragment, more preferably is a functional homolog, of any one of SEQ ID NO 02, 03, 04, 05, 06, 07, 08, 09, 10, 11, 12, 13, 14, 15, 16, 17 or 18 having at least 80%, preferably at least 85%, more preferably at least 90%, even more preferably at least 95%, most preferably at least 97%, overall sequence identity to the full-length of any one of said polypeptide with SEQ ID NO 02, 03, 04, 05, 06, 07, 08, 09, 10, 11, 12, 13, 14, 15, 16, 17 or 18, respectively, and having Neu(n)Ac synthase activity.
4. Cell according to any one of claims 1 to 3, wherein said bioproduct is an N-acetylneuraminic acid (Neu(n)Ac)-containing compound wherein (n) is 4, 5, 7, 8 or 9 or a combination thereof and wherein said cell is modified, preferably has been modified, in the expression or activity of at least one Neu(n)Ac synthase which has Neu(n)Ac synthase activity and which has PFAM domain PF03102 and/or PatricDB global family domain PGF_06907304 and which
   comprises or consists of a polypeptide sequence according to any one of SEQ ID NO 02, 03, 04, 05, 06, 07, 08, 09, 10, 11, 12, 13, 14, 15, 16, 17 or 18, or
   is a functional homolog, variant, derivative or functional fragment, preferably is a functional homolog or functional fragment, more preferably is a functional homolog, of any one of SEQ ID NO 02, 03, 04, 05, 06, 07, 08, 09, 10, 11, 12, 13, 14, 15, 16, 17 or 18 having at least 85%, preferably at least 90%, more preferably at least 95%, even more preferably at least 97%, overall sequence identity to the full-length of any one of said polypeptide with SEQ ID NO 02, 03, 04, 05, 06, 07, 08, 09, 10, 11, 12, 13, 14, 15, 16, 17 or 18, respectively, and having Neu(n)Ac synthase activity.
5. Cell according to any one of claims 1 to 4, wherein said modification comprises overexpression of an endogenous Neu(n)Ac synthase and/or introduction and expression of a homologous or heterologous Neu(n)Ac synthase.
6. Cell according to any one of claims 1 to 5, wherein said Neu(n)Ac synthase is presented to the cell in one or more gene expression modules wherein expression is regulated by one or more regulatory sequences.
7. Cell according to any one of claims 1 to 6, wherein said expression modules are integrated in the host cell's genome and/or presented to the cell on a vector comprising plasmid, cosmid, phage, liposome or virus, which is to be stably transformed into said host cell.
8. Cell according to any one of claims 1 to 7, wherein said pathway for production of said Neu(n)Ac-containing compounds comprises at least one enzyme chosen from the list comprising N-acylglucosamine 2-epimerase, UDP-N-acetylglucosamine 2-epimerase, N-acetylmannosamine-6-phosphate 2-epimerase, bifunctional UDP-GlcNAc 2-epimerase/kinase, N-acylneuraminate-9-phosphate synthetase, phosphatase, CMP sialic acid synthase, sialyltransferase.
9. Cell according to claim 8, wherein said N-acylneuraminate-9-phosphate synthetase is a polypeptide having N-acylneuraminate-9-phosphate synthetase activity and which has PFAM domain PF03102 and/or PatricDB global family domain PGF_06907304 and which comprises the sequence [DE]XGXNHXGXXXXXXXMXXX[ACPS]XXXXXXXX[KR](Xa)[KR](Xb)[DE](Xc)KXXS with SEQ ID NO 19, wherein X is any amino acid, a is 28 to 32, b is 28 to 30 and c is 14 to 16, preferably which comprises the sequence [DE]XGXNHXGXXXXXXXMXX(X, no I, L, M)[AS]XXXXXXXX[KR](Xa)[KR](Xb)[DE](Xc)KXXS with SEQ ID NO 20, wherein X is any amino acid, a is 28 to 32, b is 28 to 30 and c is 14 to 16.
10. Cell according to claim 8 or 9, wherein said N-acylneuraminate-9-phosphate synthetase is a polypeptide having N-acylneuraminate-9-phosphate synthetase activity and which has PFAM domain PF03102 and/or PatricDB global family domain PGF_06907304 and which
   comprises or consists of a polypeptide sequence according to any one of SEQ ID NO 21, 22, 23, 24, 25, 26, 27 or 28, or
   is a functional homolog, variant, derivative or functional fragment, preferably is a functional homolog or functional fragment, more preferably is a functional homolog, of any one of SEQ ID NO 21, 22, 23, 24, 25, 26, 27 or 28 having at least 80%, preferably at least 85%, more preferably at least 90%, even more preferably at least 95%, most preferably at least 97%, overall sequence identity to the full-length of any one of said polypeptide with SEQ ID NO 21, 22, 23, 24, 25, 26, 27 or 28, respectively, and having N-acylneuraminate-9-phosphate synthetase activity.
11. Cell according to claim 8, wherein said phosphatase is a HAD-like phosphatase having N-acylneuraminate-9-phosphatase activity and:
   comprising the sequence
   DXDGXXTDXXXXXXXXGXXXXXXXXXDXXXXXXXXXXXXXXX[ILV]X[ST]XXXXXXXXXRXXXL(Xa)K(Xb)GX DXXD(Xc)GXGXXR[DE] with SEQ ID NO 29, wherein X is any amino acid, a is 10 to 11, b is 21 and cis32,or
   comprising the sequence DXDXT[IL](Xa)TNGXXXXQXXK[IL](Xb)[KR]PXXX[IL][FWY](Xc)G[DN]XXXXD[ILV]XG with SEQ ID NO 31, wherein X is any amino acid, a is 110 to 160, b is 20 to 23 and c is 17.
12. Cell according to any one of claims 1 to 11, wherein said cell is capable to synthesize N-acetylmannosamine (ManNAc), N-acetylmannosamine-6-phosphate (ManNAc-6-phosphate) and/or phosphoenolpyruvate (PEP).
13. Cell according to any one of claims 1 to 12, wherein said cell is modified for enhanced synthesis and/or supply of PEP.
14. Cell according to any one of claims 1 to 13, wherein said cell is further capable to synthesize any one or more nucleotide-activated sugars.
15. Cell according to claim 14, wherein said nucleotide-activated sugar is chosen from the list comprising UDP-N-acetylglucosamine (UDP-GIcNAc), UDP-N-acetylgalactosamine (UDP-GaINAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
16. Cell according to claim 14 or 15, wherein at least one of said nucleotide-activated sugars is derived from Neu(n)Ac, comprising CMP-Neu4Ac, CMP-Neu5Ac, CMP-Neu5Ac9N₃, CMP-Neu4,5Ac₂, CMP-Neu5,7Ac₂, CMP-Neu5,9Ac₂, CMP-Neu5,7(8,9)Ac₂ and CMP-Neu5Gc.
17. Cell according to any one of claims 14 to 16, wherein said cell uses at least one of said nucleotide-activated sugars in the production of said Neu(n)Ac-containing compound.
18. Cell according to any one of claims 1 to 17, wherein said glycosyltransferase is chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
   preferably, wherein said cell is modified in the expression or activity of at least one of said glycosyltransferases,
   preferably, said fucosyltransferase is chosen from the list comprising alpha-1,2-fucosyltransferase, alpha-1,3-fucosyltransferase, alpha-1,4-fucosyltransferase and alpha-1,6-fucosyltransferase,
   preferably, said sialyltransferase is chosen from the list comprising alpha-2,3-sialyltransferase, alpha-2,6-sialyltransferase, and alpha-2,8-sialyltransferase,
   preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
   preferably, said glucosyltransferase is chosen from the list comprising alpha-glucosyltransferase, beta-1,2-glucosyltransferase, beta-1,3-glucosyltransferase and beta-1,4-glucosyltransferase,
   preferably, said mannosyltransferase is chosen from the list comprising alpha-1,2-mannosyltransferase, alpha-1,3-mannosyltransferase and alpha-1,6-mannosyltransferase,
   preferably, said N-acetylglucosaminyltransferase is chosen from the list comprising galactoside beta-1,3-N-acetylglucosaminyltransferase and beta-1,6-N-acetylglucosaminyltransferase,
   preferably, said N-acetylgalactosaminyltransferase is chosen from the list comprising alpha-1,3-N-acetylgalactosaminyltransferase.
19. Cell according to any one of claims 1 to 18, wherein said cell expresses at least one alpha-2,3-sialyltransferase which has alpha-2,3-sialyltransferase activity and which
   is the polypeptide from Pasteurella multocida with SEQ ID NO 80, or
   is a polypeptide of 268 amino acid residues long that is a functional homolog, variant, derivative or functional fragment, preferably is a functional homolog or functional fragment, more preferably is a functional homolog, of said polypeptide with SEQ ID NO 80 having at least 80%, preferably at least 85%, more preferably at least 90%, even more preferably at least 95%, most preferably at least 97%, overall sequence identity to the full-length of said polypeptide with SEQ ID NO 80 and having alpha-2,3-sialyltransferase activity,
preferably, said cell is modified in the expression or activity of any one of said alpha-2,3-sialyltransferase.
20. Cell according to any one of claims 1 to 19, wherein said Neu(n)Ac synthase is an Neu5Ac synthase and wherein the Neu(n)Ac-containing compound is a sialylated compound comprising Neu5Ac.
21. Cell according to any one of claims 1 to 20, wherein said cell comprises a fucosylation pathway comprising at least one enzyme chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase.
22. Cell according to any one of claims 1 to 21, wherein said cell comprises a galactosylation pathway comprising at least one enzyme chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase.
23. Cell according to any one of claims 1 to 22, wherein said cell comprises an N-acetylglucosaminylation pathway comprising at least one enzyme chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase.
24. Cell according to any one of claims 1 to 23, wherein said cell comprises a modification for reduced production of acetate.
25. Cell according to any one of claims 1 to 24, wherein said cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IcIR, Ion protease, glucose-specific translocating phosphotransferase enzyme IIBC component ptsG, glucose-specific translocating phosphotransferase (PTS) enzyme IIBC component malX, enzyme IIAGc, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.
26. Cell according to any one of claims 1 to 25, wherein said cell comprises a catabolic pathway for selected mono-, di- or oligosaccharides which is at least partially inactivated, the mono-, di-, or oligosaccharides being involved in and/or required for the synthesis of said Neu(n)Ac-containing compound.
27. Cell according to any one of claims 1 to 26, wherein said cell is using a precursor for the synthesis of said bioproduct, said precursor being fed to the cell from the cultivation medium.
28. Cell according to any one of claims 1 to 27, wherein said cell is producing a precursor for the synthesis of said bioproduct.
29. Cell according to any one of claims 1 to 28, wherein said cell produces 90 g/L or more of said bioproduct in the whole broth and/or supernatant and/or wherein said Neu(n)Ac-containing compound in the whole broth and/or supernatant has a purity of at least 80% measured on the total amount of bioproduct and its precursor produced by said cell in the whole broth and/or supernatant, respectively.
30. Cell according to any one of claims 1 to 29, wherein said Neu(n)Ac-containing compound is chosen from the list consisting sialic acid, a disaccharide, an oligosaccharide, a glycolipid and/or a glycoprotein, preferably chosen from the list consisting of sialic acid, a disaccharide, an oligosaccharide and/or a glycolipid, more preferably chosen from the list consisting of sialic acid, a disaccharide and/or an oligosaccharide.
31. Cell according to any one of claims 1 to 30, wherein said oligosaccharide is chosen from the list comprising a milk oligosaccharide, 0-antigen, an oligosaccharide repeat present in capsular polysaccharides, peptidoglycan, amino-sugars and Lewis-type antigen oligosaccharide, preferably said milk oligosaccharide is a mammalian milk oligosaccharide (MMO), more preferably said milk oligosaccharide is a human milk oligosaccharide (HMO).
32. Cell according to any one of claims 1 to 31, wherein the cell is capable to synthesize a mixture of oligosaccharides.
33. Cell according to any one of claims 1 to 32, wherein the cell is capable to synthesize a mixture of di-and oligosaccharides.
34. Cell according to any one of claims 1 to 33, wherein the cell is capable to synthesize a mixture of sialic acid, a di- and/or oligosaccharides.
35. Cell according to any one of claims 1 to 34, wherein said cell is a bacterium, fungus, yeast, a plant cell, an animal cell, or a protozoan cell,
   preferably said bacterium is of an Escherichia coli strain, more preferably of an Escherichia coli strain which is a K-12 strain, even more preferably the Escherichia coli K-12 strain is E. coli MG1655,
   preferably said fungus belongs to a genus chosen from the group comprising Rhizopus, Dictyostelium, Penicillium, Mucor or Aspergillus,
   preferably said yeast belongs to a genus chosen from the group comprising Saccharomyces, Zygosaccharomyces, Pichia, Komagataella, Hansenula, Yarrowia, Starmerella, Kluyveromyces or Debaromyces,
   preferably said plant cell is an algal cell or is derived from tobacco, alfalfa, rice, tomato, cotton, rapeseed, soy, maize, or corn plant,
   preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
   preferably said protozoan cell is a Leishmania tarentolae cell.
36. Cell according to any one of claims 1 to 35, wherein said cell is stably cultured in a medium.
37. Method to produce a bioproduct by a cell, the method comprising the steps of:
   1) providing a cell according to any one embodiment 1 to 36, and
   2) cultivating said cell under conditions permissive to produce said bioproduct,
   3) preferably, separating said bioproduct from said cultivation, preferably, wherein said bioproduct is a Neu(n)Ac-containing compound.
38. Method according to claim 37, the method further comprising at least one of the following steps:
   i) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor and/or acceptor feed;
   ii) Adding at least one precursor and/or acceptor feed in a continuous manner to the culture medium over the course of 1 day, 2 days, 3 days, 4 days, 5 days by means of a feeding solution;
   iii) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.; said method resulting in a bioproduct, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium, preferably wherein said bioproduct is a Neu(n)Ac-containing compound.
39. Method according to claim 38, the method further comprising at least one of the following steps:
   i) adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
   ii) adding a lactose feed in a continuous manner to the culture medium over the course of 1 day, 2 days, 3 days, 4 days, 5 days by means of a feeding solution;
   iii) adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.; said method resulting in a bioproduct produced from said lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium, preferably, wherein said bioproduct produced from said lactose comprises one or more Neu(n)Ac molecule(s).
40. Method according to claim 39, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.
41. Method according to claim 39 or 40, wherein said lactose feed is accomplished by adding lactose to the cultivation medium in a concentration, such, that throughout the production phase of the cultivation a lactose concentration of at least 5 mM, preferably 10 mM or 30 mM is obtained.
42. Method according to any one of claims 37 to 41, wherein the host cells are cultivated for at least about 60, 80, 100, or about 120 hours or in a continuous manner.
43. Method according to any one of claims 37 to 42, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.
44. Method according to any one of claims 37 to 43, wherein said cell uses at least one precursor for the synthesis of said bioproduct, preferably said cell uses two or more precursors for the synthesis of said bioproduct.
45. Method according to any one of claims 37 to 44, wherein the culture medium contains at least one compound selected from the group comprising lactose, galactose, sialic acid, fucose, GlcNAc, GaINAc, lacto-N-biose (LNB), N-acetyllactosamine (LacNAc).
46. Method according to any one of claims 37 to 45, wherein a first phase of exponential cell growth is provided by adding a carbon-based substrate, preferably glucose or sucrose, to the culture medium before the lactose is added to the culture medium in a second phase.
47. Method according to any one of claims 37 to 46, wherein said cell is producing at least one precursor for the synthesis of said bioproduct.
48. Method according to any one of claims 37 to 47, wherein said precursor for the synthesis of said bioproduct is completely converted into said bioproduct.
49. Method according to any one of claims 37 to 48, wherein the bioproduct is separated from the culture medium and/or the cell.
50. Method according to any one of claims 37 to 49, wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography.
51. Method according to any one of claims 37 to 50, wherein said method further comprises purification of said bioproduct.
52. Method according to claim 51, wherein said purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying or lyophilization.
53. Use of a cell according to any one of claims 1 to 36 for production of a bioproduct.
54. Use of a cell according to any one of claims 1 to 36 for production of a Neu(n)Ac-containing compound wherein (n) is 4, 5, 7, 8 or 9 or a combination thereof.
55. Use of a method according to any one of claims 37 to 52 for production of a bioproduct.
56. Use of a method according to any one of claims 37 to 52 for production of a Neu(n)Ac-containing compound wherein (n) is 4, 5, 7, 8 or 9 or a combination thereof.

The invention will be described in more detail in the examples. The following examples will serve as further illustration and clarification of the present invention and are not intended to be limiting.

### EXAMPLES

### Example 1. Identification of Protein Domains

An HMM is a probabilistic model called profile hidden Markov models. It characterizes a set of aligned proteins into a position-specific scoring system. Amino acids are given a score at each position in the sequence alignment according to the frequency by which they occur (Eddy, S. R. 1998. Profile hidden Markov models. Bioinformatics. 14: 755-63). HMMs have wide utility, as is clear from the numerous databases that use this method for protein classification, including Pfam, InterPro, SMART, TIGRFAM, PIRSF, PANTHER, SFLD, Superfamily and Gene3D.

HMMsearch from the HMMER package 3.2.1 (http://hmmer.org/) as released on₁₃th June 2019 can use this HMM to search sequence databases for sequence homologs. Sequence databases that can be used are for example, but not limited to the NCBI nr Protein Database (NR; https://www.ncbi.nlm.nih.gov/protein), UniProt Knowledgebase (UniProtKB, https://www.uniprot.org/help/uniprotkb) and the SWISS-PROT database (https://web.expasy.org/docs/swiss-prot_guideline.html). Neu(n)Ac synthases were classified based on InterPro 75.0 (https://www.ebi.ac.uk/interpro/) as released on Jul. 4, 2019 and PFAM domains using Pfam 32.0 (https://pfam.xfam.org/) as released on Sept 2018. The Pfam and InterPro databases are a large collection of protein families. Other protein domains like SMART (http://smart.embl-heidelberg.de/), TIGRFAM (https://www.jcvi.org/tigrfams), PIRSF (https://proteininformationresource.org/pirwww/dbinfo/pirsf.shtml), PANTHER (http://pantherdb.org/), SFLD (http://sfld.rbvi.ucsf.edu/archive/django/index.html), Superfamily (http://supfam.org/) and Gene3D (http://gene3d.biochem.ucl.ac.uk/Gene3D/), NCBI Conserved Domains (https://www.ncbi.nlm.nih.gov/Structure/cdd/wrpsb.cgi) can also be used. Identification of the PFAM domains was done by an online search on https://pfam.xfam.org/search #tabview=tabl as released on Sept 2018. The HMM for the obtained family was downloaded in ‘Curation & model’. HMMsearches with this model to the protein databases will identify new family members. Sequences comprising the InterPro hit can also be downloaded from the PFAM website.

Identification of the InterPro (super)families, domains and sites was done by using the online tools on https://www.ebi.ac.uk/interpro/or a standalone version of InterProScan (https://www.ebi.ac.uk/interpro/download.html), both based on InterPro 75.0 as released on Jul, 4, 2019. InterPro is a composite database combining the information of many databases of protein motifs and domains. The HMM of the InterPro domain and/or (super)families can be obtained from InterProScan and can be used to identify new family members in the protein databases. Sequences comprising the InterPro hit can also be downloaded from the InterPro website (‘Protein Matched’) or can be queried on the UniProt website (https://www.uniprot.org).

### Example 2: Calculation of Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e. spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e. over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e. spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith TF, Waterman MS (1981) J. Mol. Biol 147(1); 195-7).

### Example 3: Identification of Neu(n)Ac Synthase Genes

Neu(n)Ac synthase genes can be obtained from sequence databases like PATRIC (https://www.patricbrc.org/), Uniprot (https://www.uniprot.org/), NCBI nr or nt databases (https://www.ncbi.nlm.nih.gov/) and others. PATRIC (https://www.patricbrc.org/) is an integration of different types of data and software tools that support research on bacterial pathogens. This example describes how to extract genes comprising the [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01 and represented by the PatricDB global family domain PGF_06907304. Members of this family were extracted using the PATRIC command-line interface (https://docs.patricbrc.org/cli_tutorial/index.html). A regex search with the domain was performed with a custom script in python (https://www.python.org/; https://docs.python.org/3/library/re.html). 5166 identifiers were extracted on 23 Feb. 2021. Amino acid sequences were filtered on completeness (from start codon to stop codon) and clustered using CD-HIT (http://weizhongli-lab.org/cd-hit/) with a sequence identity threshold of 80%. The representative sequences are the next 305 identifiers: fig |1002804.6.peg.547, fig |100884.13.peg.450, fig |1038844.30.peg.4934, fig |1051651.3.peg.3205, fig |1055528.4.peg.829, fig |1062.16.peg.1137, fig |1063.32.peg.2, fig |1066.21.peg.3289, fig |1071054.3.peg.3038, fig |1078087.3.peg.2199, fig| 1098.5.peg.1527, fig 110321.109.peg.452, fig |1120935.3.peg.6089, fig |1120965.3.peg.2870, fig |1121102.3.peg.1353, fig |1121866.8.peg.1014, fig |1122159.4.peg.3850, fig |1122938.3.peg.280, fig |1122991.5.peg.2486, fig |1123289.3.peg.1544, fig |1123490.3.peg.901, fig |1123502.3.peg.977, fig |1123755.5.peg.991, fig |1131272.3.peg.445, fig |1167006.5.peg.3367, fig |1167637.4.peg.2723, fig |1191687.11.peg.701, fig |1203593.3.peg.1211, fig |121719.18.peg.1483, fig |1230338.3.peg.604, fig |1232673.3.peg.665, fig |1236514.3.peg.5464, fig |1236959.3.peg.2826, fig |1238198.3.peg.4318, fig| 123841.6.peg.11, fig |1244531.6.peg.922, fig |1249978.3.peg.2463, fig |1262761.3.peg.55, fig |1262830.3.peg.1125, fig |1263550.3.peg.3253, fig |1264.4.peg.3685, fig |1265827.4.peg.2143, fig |1278971.3.peg.967, fig |1281017.3.peg.2297, fig |128944.3.peg.1532, fig |1295392.6.peg.2032, fig |1304887.3.peg.2105, fig |1304902.6.peg.4111, fig |1307.262.peg.1750, fig |1326.4.peg.580, fig |132932.4.peg.789, fig |1349.576.peg.1564, fig |1354263.4.peg.1545, fig |1357398.3.peg.1891, fig |1380600.3.peg.824, fig |1404367.6.peg.3984, fig |1408287.3.peg.210, fig |1410612.3.peg.1243, fig |1410627.3.peg.1777, fig |1411316.19.peg.446, fig |142586.12.peg.2167, fig |142586.1785.peg.691, fig |1429889.3.peg.2679, fig |1471452.3.peg.1260, fig |1471472.3.peg.1861, fig |1471528.3.peg.1140, fig |1476195.3.peg.1667, fig |1499679.3.peg.1909, fig |1500268.4.peg.2015, fig |1503961.5.peg.1771, fig |1503961.5.peg.5506, fig |1506.455.peg.2765, fig |1520802.5.peg.758, fig |1521931.3.peg.1058, fig |152507.1091.peg.2096, fig |152509.1038.peg.219, fig |152509.176.peg.270, fig |152509.200.peg.499, fig |152509.254.peg.178, fig |152509.280.peg.778, fig |152509.299.peg.128, fig |152509.317.peg.262, fig |152509.78.peg.172, fig |1528787.3.peg.5741, fig |1529069.3.peg.771, fig |153493.10.peg.569, fig |153809.403.peg.1128, fig |1548018.3.peg.2565, fig |158836.1033.peg.707, fig |1612.51.peg.2241, fig |1618405.4.peg.579, fig |1618511.4.peg.1174, fig |1618608.4.peg.701, fig |1618811.4.peg.13, fig |1629713.5.peg.5017, fig |1632858.5.peg.2548, fig |165185.186.peg.145, fig |1651968.3.peg.1506, fig |1660074.3.peg.176, fig |1667051.1649.peg.455, fig |1667051.2072.peg.1080, fig |1667051.527.peg.685, fig |1667051.856.peg.326, fig |1667051.973.peg.458, fig |1703402.5.peg.672, fig |1713224.3.peg.4337, fig |1727163.4.peg.2932, fig |172733.1895.peg.1453, fig |1729725.3.peg.1203, fig |1735162.10.peg.921, fig |1736373.3.peg.2598, fig |1736447.3.peg.3772, fig |1736490.3.peg.4357, fig |1740262.3.peg.2403, fig |1758176.3.peg.527, fig |1765022.11.peg.2795, fig |1797193.3.peg.4233, fig |1797315.3.peg.1355, fig |1797393.3.peg.2372, fig |1797403.3.peg.835, fig |1797915.3.peg.1834, fig |1797968.3.peg.1910, fig |1798263.3.peg.584, fig |1798268.3.peg.697, fig |1798314.3.peg.1758, fig |1798325.3.peg.478, fig |1798417.3.peg.2202, fig |1798663.3.peg.62, fig |1798683.3.peg.1004, fig |1801946.3.peg.1213, fig |1802304.3.peg.776, fig |1802384.3.peg.808, fig |180441.4.peg.3020, fig |1813019.44.peg.319, fig |181674.329.peg.984, fig |1817801.7.peg.1665, fig |1838286.3.peg.2066, fig |1861.4.peg.2593, fig |1869227.373.peg.512, fig |1869227.456.peg.1784, fig |1870990.3.peg.1620, fig |1871037.156.peg.940, fig |1871037.182.peg.718, fig |1871037.218.peg.1968, fig |1871070.14.peg.4116, fig|1879010.6632.peg.434, fig|1897043.3.peg.2266, fig|1897630.3.peg.4093, fig |1898103.33.peg.1215, fig |1898104.528.peg.105, fig |1898203.1837.peg.1385, fig |1898203.2140.peg.2206, fig |1898203.2142.peg.1585, fig |1898203.3.peg.3294, fig |1898203.83.peg.2892, fig |1898205.3894.peg.1218, fig |1898207.3640.peg.188, fig |1898207.3841.peg.406, fig |1902409.7.peg.1786, fig |1904441.117.peg.2724, fig |1904441.306.peg.2282, fig |1904441.319.peg.1012, fig |1904441.57.peg.2253, fig |1909294.1059.peg.4719, fig |1909294.250.peg.2233, fig |1913989.525.peg.756, fig |1917019.3.peg.1281, fig |1926876.3.peg.2711, fig |1936995.3.peg.2504, fig |1946156.3.peg.1473, fig |1946312.3.peg.2491, fig |1947389.3.peg.2953, fig |1948890.13.peg.1302, fig |194923.51.peg.476, fig |195.447.peg.1271, fig |195045.59.peg.232, fig |1951479.3.peg.1084, fig |1951543.3.peg.934, fig |1951832.3.peg.766, fig |1961389.3.peg.1983, fig |1965557.3.peg.2146, fig |1970510.3.peg.530, fig |1973990.3.peg.490, fig |1973999.3.peg.414, fig |1974005.3.peg.33, fig |1974785.3.peg.457, fig |1975096.3.peg.589, fig |1978231.219.peg.3848, fig |1978412.4.peg.3849, fig |2013726.3.peg.388, fig |2013763.3.peg.1707, fig |2014542.3.peg.2964, fig |2015557.3.peg.1744, fig |202.12.peg.1481, fig |2021368.19.peg.2645, fig |2024889.18.peg.1866, fig |2026724.1106.peg.3502, fig |2026724.200.peg.6383, fig |2026734.222.peg.173, fig |2026742.89.peg.2051, fig |2026763.159.peg.1925, fig |2026774.61.peg.261, fig |2026779.78.peg.1507, fig |2026780.361.peg.1890, fig |2026791.71.peg.104, fig |2026887.129.peg.3163, fig |2030927.473.peg.2108, fig |2030927.524.peg.207, fig |2030927.667.peg.2608, fig |2035772.18.peg.1320, fig |2035772.264.peg.800, fig |2035772.275.peg.900, fig |2040651.3.peg.68, fig |2042961.3.peg.1534, fig |2043167.3.peg.427, fig |2044940.15.peg.1114, fig |2048255.3.peg.1736, fig |2049431.34.peg.937, fig |2049433.30.peg.3322, fig |2049433.7.peg.3086, fig |2053044.3.peg.1515, fig |2053617.19.peg.785, fig |2053634.101.peg.161, fig |207559.7.peg.3187, fig |208199.4.peg.2141, fig |208549.72.peg.3116, fig |2093811.128.peg.948, fig |2170728.3.peg.2380, fig |220137.23.peg.916, fig |2219558.3.peg.1000, fig |243904.133.peg.1246, fig |2448778.22.peg.1348, fig |2490856.3.peg.961, fig |2497619.14.peg.117, fig |253161.68.peg.484, fig |2569543.3.peg.4125, fig |2588536.27.peg.841, fig |2604158.3.peg.4421, fig |2607825.3.peg.11214, fig |263474.3.peg.560, fig |2656915.3.peg.5209, fig |2711156.3.peg.2713, fig |2731251.3.peg.3034, fig |2740185.3.peg.3368, fig |2750080.10.peg.516, fig |2750080.3.peg.667, fig |2750080.5.peg.124, fig |2756.14.peg.1483, fig |2760054.3.peg.2498, fig |2780076.3.peg.449, fig |281031.13.peg.1088, fig |281031.138.peg.18, fig |28200.10.peg.698, fig |29391.32.peg.1415, fig |29410.5.peg.472, fig |296218.3.peg.948, fig |310575.5.peg.2356, fig |31910.4.peg.371, fig |331630.40.peg.1600, fig |37372.27.peg.428, fig |388413.5.peg.9, fig |435880.7.peg.3933, fig |435906.4.peg.2580, fig |43657.14.peg.3706, fig |43658.6.peg.3927, fig |442714.3.peg.2766, fig |446043.5.peg.671, fig |453575.4.peg.3610, fig |46206.5.peg.2009, fig |49280.10.peg.1576, fig |51161.3.peg.1247, fig |522484.4.peg.276, fig |553385.4.peg.2463, fig |555950.3.peg.1285, fig |587909.3.peg.1713, fig |59620.1573.peg.14, fig |59620.1655.peg.3594, fig |626932.184.peg.2675, fig |648174.107.peg.900, fig |648174.118.peg.198, fig |1648174.164.peg.1309, fig |648174.193.peg.631, fig |648174.208.peg.126, fig |648174.219.peg.818, fig |648174.233.peg.187, fig |648174.329.peg.501, fig |1648174.387.peg.83, fig |648174.506.peg.471, fig |648174.512.peg.430, fig |648174.513.peg.458, fig |69360.3.peg.3235, fig |750.10.peg.1591, fig |797277.3.peg.2464, fig |86027.621.peg.1978, fig |86473.95.peg.552, fig |869962.3.peg.2770, fig |87012.4.peg.1660, fig |91750.123.peg.781, fig |91750.394.peg.421, fig |91750.45.peg.463, fig |1932004.23.peg.1055, fig |943346.3.peg.444, fig |945543.3.peg.4068, fig |964.13.peg.4412, fig |989403.3.peg.4566.

### Example 4. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH4CI, 5.00 g/L (NH4)2SO4, 2.993 g/L KH2PO4, 7.315 g/L K2HPO4, 8.372 g/L MOPS, 0.5 g/L NaCl, 0.5 g/L MgSO4.7H20, 30 g/L sucrose or 30 g/L glycerol, 1 ml/L vitamin solution, 100 pl/L molybdate solution, and 1 mL/L selenium solution. As specified in the respective examples, 0.30 g/L sialic acid, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeC12.4H20, 5 g/L CaCl2.2H20, 1.3 g/L MnC12.2H20, 0.38 g/L CuC12.2H20, 0.5 g/L CoC12.6H20, 0.94 g/L ZnC12, 0.0311 g/L H3BO4, 0.4 g/L Na2EDTA.2H20 and 1.01 g/L thiamine.HCl. The molybdate solution contained 0.967 g/L NaMoO4.2H20. The selenium solution contained 42 g/L Seo2.

The minimal medium for fermentations contained 6.75 g/L NH4CI, 1.25 g/L (NH4)2SO4, 2.93 g/L KH2PO4 and 7.31 g/L KH2PO4, 0.5 g/L NaCl, 0.5 g/L MgSO4.7H20, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc and/or 20 g/L LNB were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F-, phi80dlacZAM15, A(lacZYA-argF) U169, deoR, recA1, endA1, hsdR17(rk−, mk+), phoA, supE44, lambda-, thi-1, gyrA96, re/A1) bought from Invitrogen.

### Strains and Mutations

Escherichia coli K12 MG1655 [X-, F-, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an ODroonm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 uL of cells and 10-100 ng of linear double-stranded-DNA product by using a Gene Pulser™ (BioRad) (600 Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature- sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In one example for sialic acid production, the mutant strain was derived from E. coli K12 MG1655 comprising genomic knock-ins of constitutive transcriptional units containing a glucosamine 6-phosphate N-acetyltransferase like e.g. GNA1 from Saccharomyces cerevisiae with SEQ ID NO 34, an N-acetylglucosamine 2-epimerase like e.g. AGE from Bacteroides ovatus with SEQ ID NO 35 and any one or more N-acetylneuraminate (Neu(n)Ac) synthases chosen from the list comprising SEQ ID NO 02, 03, 04, 05, 06, 07, 08, 09, 10, 11, 12, 13, 14, 15, 16, 17 and 18.

Alternatively and/or additionally, sialic acid production can be obtained by genomic knock-ins of constitutive transcriptional units containing an UDP-N-acetylglucosamine 2-epimerase like e.g. NeuC from Campylobacter jejuni with SEQ ID NO 39 and any one or more Neu(n)Ac synthases chosen from the list comprising SEQ ID NO 02, 03, 04, 05, 06, 07, 08, 09, 10, 11, 12, 13, 14, 15, 16, 17 and 18.

Alternatively and/or additionally, sialic acid production can be obtained by genomic knock-ins of constitutive transcriptional units containing a phosphoglucosamine mutase like e.g. glmM from E. coli with SEQ ID NO 37, an N-acetylglucosamine-1-phosphate uridyltransferase/glucosamine-1-phosphate acetyltransferase like e.g. glmU from E. coli with SEQ ID NO 38, an UDP-N-acetylglucosamine 2-epimerase like e.g. NeuC from Campylobacter jejuni with SEQ ID NO 39 and any one or more Neu(n)Ac synthases chosen from the list comprising SEQ ID NO 02, 03, 04, 05, 06, 07, 08, 09, 10, 11, 12, 13, 14, 15, 16, 17 and 18.

Alternatively and/or additionally, sialic acid production can be obtained by genomic knock-ins of constitutive transcriptional units containing a bifunctional UDP-GIcNAc 2-epimerase/N-acetylmannosamine kinase like e.g. from Mus musculus (strain C57BL/6J) with SEQ ID NO 33, an N-acylneuraminate-9-phosphate synthetase chosen from the list comprising SEQ ID NO 21, 22, 23, 24, 25, 26, 27 and 28, and an N-acylneuraminate-9-phosphatase like e.g. from Candidatus Magnetomorum sp. HK-1 with SEQ ID NO 30 or from Bacteroides thetaiotaomicron (strain ATCC 29148) with SEQ ID NO 32.

Alternatively and/or additionally, sialic acid production can be obtained by genomic knock-ins of constitutive transcriptional units containing a phosphoglucosamine mutase like e.g. glmM from E. coli with SEQ ID NO 37, an N-acetylglucosamine-1-phosphate uridyltransferase/glucosamine-1-phosphate acetyltransferase like e.g. glmU from E. coli with SEQ ID NO 38, a bifunctional UDP-GIcNAc 2-epimerase/N-acetylmannosamine kinase like e.g. from M. musculus (strain C57BL/6J) with SEQ ID NO 33, an N-acylneuraminate-9-phosphate synthetase chosen from the list comprising SEQ ID NO 21, 22, 23, 24, 25, 26, 27 and 28, and an N-acylneuraminate-9-phosphatase like e.g. from Candidatus Magnetomorum sp. HK-1 with SEQ ID NO 30 or from Bacteroides thetaiotaomicron (strain ATCC 29148) with SEQ ID NO 32. Sialic acid production can further be optimized in the mutant E. coli strain with genomic knock-outs of the E. coli genes comprising any one or more of nagA, nagB, nagC, nagD, nagE, nanA, nanE, nanK, manX, manYand manZ as described in WO18122225, and/or genomic knock-outs of the E. coligenes comprising any one or more of poxB, ldhA, adhE, aldB, pf/A, pflC, ybiY, ackA and/or pta, and with genomic knock-ins of constitutive transcriptional units comprising any one or more of an L-glutamine-D-fructose-6-phosphate aminotransferase like e.g. the mutant glmS*54 from E. coli with SEQ ID NO 36 (differing from the wild-type E. coli glmS by an A39T, an R250C and an G472S mutation), preferably a phosphatase like any one or more of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225 and an acetyl-CoA synthetase like e.g. acs from E. coli with SEQ ID NO 79.

For sialylated oligosaccharide production, said sialic acid production strains further need to express one or more copies of an N-acylneuraminate cytidylyltransferases like e.g. NeuA from Pasteurella multocida with SEQ ID NO 40, NeuA from C. jejuni with SEQ ID NO 64 or NeuA from Haemophilus influenzae with SEQ ID NO 65, and one or more copies of a beta-galactoside alpha-2,3-sialyltransferase, e. g. chosen from the list comprising SEQ ID NO 41 (PmultST2) from P. multocida subsp. multocida str. Pm70, SEQ ID NO 42 (NmeniST3) from N. meningitidis and SEQ ID NO 80 (PmultST3) from P. multocida, a beta-galactoside alpha-2,6-sialyltransferase, such as the one comprising SEQ ID NO 43 (PdST6) from Photobacterium damselae and SEQ ID NO 44 (P-JT-ISH-224-ST6) from Photobacterium sp. JT-ISH-224, and/or an alpha-2,8-sialyltransferase, such as e.g. from Mus musculus with SEQ ID NO 45. Constitutive transcriptional units of the N-acylneuraminate cytidylyltransferases and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g. the E. coli LacY with SEQ ID NO 46. All mutant strains producing sialic acid, CMP-sialic acid and/or sialylated oligosaccharides could optionally be adapted for growth on sucrose via genomic knock-ins of constitutive transcriptional units containing a sucrose transporter (CscB), e.g. from E. coli W with SEQ ID NO 47, a fructose kinase (Frk) e.g. originating from Z. mobilis with SEQ ID NO 48 and a sucrose phosphorylase e.g. originating from B. adolescentis with SEQ ID NO 49.

For GFP-fucose production in the E. coli strains producing sialic acid, the mutant strains in these examples were further modified comprising knock-outs of the E. coli wcaJ and thyA genes and genomic knock-ins of constitutive transcriptional units containing a sucrose transporter like e.g. CscB from E. coli W with SEQ ID NO 47, a fructose kinase like e.g. Frk originating from Zymomonas mobilis with SEQ ID NO 48 and a sucrose phosphorylase (SP) like e.g. from Bifidobacterium adolescentis with SEQ ID NO 49. For production of fucosylated oligosaccharides, the mutant GDP-fucose production strain was additionally modified with expression plasmids comprising constitutive transcriptional units for an alpha-1,2-fucosyltransferase like e.g. HpFutC from H. pylori with SEQ ID NO 50 and/or an alpha-1,3-fucosyltransferase like e.g. HpFucT from H. pylori with SEQ ID NO 51 and with a constitutive transcriptional unit for the E. coli thyA with SEQ ID NO 52 as selective marker. The constitutive transcriptional units of the fucosyltransferase genes could also be present in the mutant E. coli strain via genomic knock-ins. GDP-fucose production can further be optimized in the mutant E. coli strain by genomic knock-outs of any one or more of the E. coli genes comprising g/gC, agp, pfkA, pfkB, pgi, arcA, ic/R, pgi and/on as described in WO2016075243 and WO2012007481. GDP-fucose production can additionally be optimized comprising genomic knock-ins of constitutive transcriptional units for a mannose-6-phosphate isomerase like e.g. manA from E. coli with SEQ ID NO 53, a phosphomannomutase like e.g. manB from E. coli with SEQ ID NO 54, a mannose-1-phosphate guanylyltransferase like e.g. manC from E. coli with SEQ ID NO 55, a GDP-mannose 4,6-dehydratase like e.g. gmd from E. coli with SEQ ID NO 56 and a GDP-L-fucose synthase like e.g. fcl from E. coli with SEQ ID NO 57. GDP-fucose production can also be obtained by genomic knock-outs of the E. coli fucK and fuc/genes and genomic knock-ins of constitutive transcriptional units containing a fucose permease like e.g. fucP from E. coli with SEQ ID NO 58 and a bifunctional fucose kinase/fucose-1-phosphate guanylyltransferase like e.g. fkp from Bacteroides fragilis with SEQ NO ID 59. If the mutant strains producing sialic acid and GDP-fucose were intended to make fucosylated lactose structures, the strains were additionally modified with genomic knock-outs of the E. coli LacZ, LacY and LacA genes and with a genomic knock-in of a constitutive transcriptional unit for a lactose permease like e.g. the E. coli LacY with SEQ ID NO 46. Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g. NeuA from P. multocida with SEQ ID NO 40, NeuA from C. jejuni with SEQ ID NO 64 or NeuA from H. influenzae with SEQ ID NO 65, and one or more copies of a beta-galactoside alpha-2,3-sialyltransferase like e.g. SEQ ID NO 41 (PmuItST2) from P. multocida subsp. multocida str. Pm70, SEQ ID NO 42 (NmeniST3) from N. meningitidis or SEQ ID NO 80 (PmultST3) from P. multocida, a beta-galactoside alpha-2,6-sialyltransferase like e.g. SEQ ID NO 43 (PdST6) from Photobacterium damselae and/or SEQ ID NO 44 (P-JT-ISH-224-ST6) from Photobacterium sp. JT-ISH-224 and/or an alpha-2,8-sialyltransferase like e.g. from M. musculus with SEQ ID NO 45.

For production of LN3 (GIcNAc-b1,3-Gal-b1,4-Glc) in the E. coli strains producing sialic acid, the mutant strains in these examples were further modified comprising genomic knock-outs of the E. coli LacZ, LacY and LacA genes and genomic knock-ins of constitutive transcriptional units for a lactose permease like e.g. the E. coli LacY with SEQ ID NO 46 and a galactoside beta-1,3-N-acetylglucosaminyltransferase like e.g. LgtA from N. meningitidis with SEQ ID NO 60.

For production of LN3-derived oligosaccharides like lacto-N-tetraose (LNT) and lacto-N-neotetraose (LNnT) the mutant LN3 producing strains were further modified with a constitutive transcriptional unit delivered to the strain either via genomic knock-in or from an expression plasmid for an N-acetylglucosamine beta-1,3-galactosyltransferase like e.g. WbgO from E. coli 055:H7 with SEQ ID NO 61 to produce LNT or for an N-acetylglucosamine beta-1,4-galactosyltransferase like e.g. LgtB from N. meningitidis with SEQ ID NO 62 to produce LNnT. Optionally, multiple copies of the galactoside beta-1,3-N-acetylglucosaminyltransferase, the N-acetylglucosamine beta-1,3-galactosyltransferase and/or the N-acetylglucosamine beta-1,4-galactosyltransferase encoding genes could be added to the mutant E. coli strains. In addition, the strains can optionally be modified for enhanced UDP-galactose production with genomic knock-outs of the E. coli ushA and ga/T genes. The mutant E. coli strains can also optionally be adapted with a genomic knock-in of a constitutive transcriptional unit for an UDP-glucose-4-epimerase like e.g. galE from E. coli with SEQ ID NO 63. Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g. NeuA from P. multocida with SEQ ID NO 40, NeuA from C. jejuni with SEQ ID NO 64 or NeuA from H. influenzae with SEQ ID NO 65, and one or more copies of a beta-galactoside alpha-2,3-sialyltransferase like e.g. SEQ ID NO 41 (PmultST2) from P. multocida subsp. multocida str. Pm70, SEQ ID NO 42 (NmeniST3) from N. meningitidis or SEQ ID NO 80 (PmultST3) from P. multocida, a beta-galactoside alpha-2,6-sialyltransferase like e.g. SEQ ID NO 43 (PdST6) from Photobacterium damselae and/or SEQ ID NO 44 (P-JT-ISH-224-ST6) from Photobacterium sp. JT-ISH-224 and/or an alpha-2,8-sialyltransferase like e.g. from M. musculus with SEQ ID NO 45. The mutant E. coli strains can also optionally be adapted for growth on sucrose via genomic knock-ins of constitutive transcriptional units containing a sucrose transporter like e.g. CscB from E. coli W with SEQ ID NO 47, a fructose kinase like e.g.

Frk originating from Z. mobilis with SEQ ID NO 48 and a sucrose phosphorylase like e.g. from B. adolescentis with SEQ ID NO 49.

Preferably but not necessarily, the glycosyltransferases were N-terminally fused to an MBP-tag to enhance their solubility (Fox et al., Protein SO. 2001, 10(3), 622-630).

All constitutive promoters, UTRs and terminator sequences originated from the libraries described by Mutalik et al. (Nat. Methods 2013, No. 10, 354-360) and Cambray et al. (Nucleic Acids Res. 2013, 41(9), 5139-5148). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in α 1:1 ratio with 70% glycerol).

TABLE 1
Overview of SEQ ID NOs described in the present invention

| | | | | Country of origin |
|---|---|---|---|---|
| SEQ | | | | of digital sequence |
| ID NO | Name/identifier | Organism | Origin | information |
| 01 | Neu(n)Ac-specific domain | Artificial sequence | Synthetic | N.A. |
| 02 | CsNeuB | Paeniclostridium sordellii UMC1 | Synthetic | USA |
| 03 | FIG.\|562.18756.peg.2418 | Escherichia coli strain MOD1-EC5561 | Synthetic | USA |
| 04 | FIG.\|2675155.3.peg.2362 | Providencia sp. wls1916 | Synthetic | Poland |
| 05 | FIG.\|1917441.3.peg.1268 | Streptococcus ruminantium strain | Synthetic | Japan |
| | | GUT187T | | |
| 06 | FIG.\|87012.4.peg.1660 | Helicobacter mesocricetorum strain | Synthetic | Belgium |
| | | 87012_3 | | |
| 07 | FIG.\|202.15.peg.1127 | Campylobacter mucosalis strain | Synthetic | United Kingdom |
| | | ATCC 43264 | | |
| 08 | FIG.\|43657.14.peg.3706 | Pseudoalteromonas luteoviolacea | Synthetic | USA |
| | | strain IPB1 | | |
| 09 | FIG.\|1264.4.peg.3685 | Ruminococcus albus strain AR67 | Synthetic | Unknown |
| 10 | FIG.\|1429889.3.peg.2679 | Brevibacillus borstelensis cifa_chp40 | Synthetic | Unknown |
| 11 | FIG.\|1797193.3.peg.4233 | Acidobacteria bacterium | Synthetic | USA |
| | | RIFCSPLOWO2_12_FULL_67_14b | | |
| 12 | FIG.\|1797315.3.peg.1355 | Bacteroidetes bacterium GWA2_31_9b | Synthetic | USA |
| 13 | FIG.\|180441.4.peg.3020 | Flavobacterium limicola | Synthetic | Japan |
| 14 | FIG.\|181674.329.peg.984 | uncultured Methylococcaceae bacterium | Synthetic | Switzerland |
| | | strain Loc080925-8-8m_bin-121 | | |
| 15 | FIG.\|1366046.3.peg.1186 | Rhodobacteraceae bacterium HIMB11 | Synthetic | USA |
| 16 | FIG.\|28200.10.peg.698 | Arcobacter skirrowii strain L405 | Synthetic | Canada |
| 17 | FIG.\|49280.10.peg.1576 | Gelidibacter algens | Synthetic | Unknown |
| 18 | FIG.\|648174.329.peg.501 | uncultured SAR86 cluster bacterium | Synthetic | Unknown |
| | | strain AG-909-A08 | | |
| 19 | N-acylneuraminate-9- | Artificial sequence | Synthetic | N.A. |
| | phosphate synthetase- | | | |
| | specific domain | | | |
| 20 | N-acylneuraminate-9- | Artificial sequence | Synthetic | N.A. |
| | phosphate synthetase- | | | |
| | specific domain | | | |
| 21 | FIG.\|1811704.3.peg.3049 | Syntrophorhabdus sp. PtaU1.Bin058 | Synthetic | USA |
| 22 | FIG.\|1852860.3.peg.1472 | Chloroflexi bacterium ADurb.Bin344 | Synthetic | USA |
| 23 | FIG.\|1948669.3.peg.2060 | Ignavibacteria bacterium UBA2353 | Synthetic | Australia |
| 24 | FIG.\|1948687.3.peg.971 | Ignavibacteria bacterium UBA7675 | Synthetic | Germany |
| 25 | FIG.\|2026887.211.peg.1661 | Nitrospirae bacterium strain | Synthetic | USA |
| | | NC_groundwater_1772_Pr3_B-0.1um_56_14 | | |
| 26 | FIG.\|2044940.62.peg.600 | Desulfobacterales bacterium strain | Synthetic | USA |
| | | NC_groundwater_1491_Pr4_B-0.1um_43_5 | | |
| 27 | FIG.\|34034.187.peg.1119 | uncultured delta proteobacterium | Synthetic | Finland |
| | | strain AM-lipid-02-D9_bin-1227 | | |
| 28 | FIG.\|995050.3.peg.744 | Desulfobaculum xiamenense | Synthetic | Unknown |
| 29 | N-acylneuraminate-9- | Artificial sequence | Synthetic | N.A. |
| | phosphatase-specific domain | | | |
| 30 | KPA15328.1 | Candidatus Magnetomorum sp. HK-1 | Synthetic | Germany |
| 31 | N-acylneuraminate-9- | Artificial sequence | Synthetic | N.A. |
| | phosphatase-specific domain | | | |
| 32 | 2-keto-3-deoxy-D-glycero- | Bacteroides thetaiotaomicron | Synthetic | Unknown |
| | D-galacto-9-phosphonononic | | | |
| | acid phosphatase | | | |
| 33 | GNE | Mus musculus (strain C57BL/6J) | Synthetic | USA |
| 34 | GNA1 | Saccharomyces cerevisiae | Synthetic | USA |
| 35 | AGE | Bacteroides ovatus | Synthetic | USA |
| 36 | glmS*54 | Escherichia coli K-12 MG1655 | Synthetic | USA |
| 37 | glmM | Escherichia coli K-12 MG1655 | Synthetic | USA |
| 38 | glmU | Escherichia coli K-12 MG1655 | Synthetic | USA |
| 39 | neuC | Campylobacter jejuni | Synthetic | USA |
| 40 | neuA | Pasteurella multocida | Synthetic | USA |
| 41 | alpha-2,3-sialyltransferase | Pasteurella multocida subsp. | Synthetic | Unknown |
| | | multocida str. Pm70 | | |
| 42 | alpha-2,3-sialyltransferase | Neisseria meningitidis | Synthetic | United Kingdom |
| 43 | alpha-2,6-sialyltransferase | Photobacterium damselae | Synthetic | Japan |
| 44 | alpha-2,6-sialyltransferase | Photobacterium sp. JT-ISH-224 | Synthetic | Japan |
| 45 | alpha-2,8-sialyltransferase | Mus musculus | Synthetic | USA |
| 46 | LacY | Escherichia coli K-12 MG1655 | Synthetic | USA |
| 47 | CscB | Escherichia coli W | Synthetic | USA |
| 48 | Frk | Zymomonas mobilis | Synthetic | United Kingdom |
| 49 | BaSP | Bifidobacterium adolescentis | Synthetic | Germany |
| 50 | HpFutC | Helicobacter pylori UA1234 | Synthetic | United Kingdom |
| 51 | HpFucT | Helicobacter pylori UA1234 | Synthetic | United Kingdom |
| 52 | thyA | Escherichia coli K-12 MG1655 | Synthetic | USA |
| 53 | manA | Escherichia coli K-12 MG1655 | Synthetic | USA |
| 54 | manB | Escherichia coli K-12 MG1655 | Synthetic | USA |
| 55 | manC | Escherichia coli K-12 MG1655 | Synthetic | USA |
| 56 | gmd | Escherichia coli K-12 MG1655 | Synthetic | USA |
| 57 | fcl | Escherichia coli K-12 MG1655 | Synthetic | USA |
| 58 | fucP | Escherichia coli K-12 MG1655 | Synthetic | USA |
| 59 | fkp | Bacteroides fragilis NCTC 9343 | Synthetic | United Kingdom |
| 60 | IgtA | Neisseria meningitidis | Synthetic | United Kingdom |
| 61 | wbgO | Escherichia coli O55:H7 | Synthetic | Germany |
| 62 | IgtB | Neisseria meningitidis MC58 | Synthetic | United Kingdom |
| 63 | galE | Escherichia coli K-12 MG1655 | Synthetic | USA |
| 64 | NeuA | Campylobacter jejuni | Synthetic | USA |
| 65 | NeuA | Haemophilus influenzae | Synthetic | USA |
| 66 | nanT | Escherichia coli K-12 MG1655 | Synthetic | USA |
| 67 | AcNeuB | Aeromonas caviae | Synthetic | USA |
| 68 | CkNeuB | Candidatus koribacter versatilis | Synthetic | USA |
| 69 | LpNeuB | Legionella pneumophila | Synthetic | USA |
| 70 | MjNeuB | Methanocaldococcus jannaschii | Synthetic | Unknown |
| 71 | MvNeuB | Moritella viscosa | Synthetic | Norway |
| 72 | LAC12 | Kluyveromyces lactis | Synthetic | USA |
| 73 | ppsA | Escherichia coli K-12 MG1655 | Synthetic | USA |
| 74 | PCK | Corynebacterium glutamicum | Synthetic | Denmark |
| 75 | pcka | Escherichia coli K-12 MG1655 | Synthetic | USA |
| 76 | eda | Escherichia coli K-12 MG1655 | Synthetic | USA |
| 77 | maeA | Escherichia coli K-12 MG1655 | Synthetic | USA |
| 78 | maeB | Escherichia coli K-12 MG1655 | Synthetic | USA |
| 79 | acs | Escherichia coli K-12 MG1655 | Synthetic | USA |
| 80 | alpha-2,3-sialyltransferase | Pasteurella multocida | Synthetic | USA |

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in α 1 L or 2.5 L shake flask and incubated for 24 h at 37° C. on an orbital shaker at 200 rpm. A 5 L bioreactor (having α 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH40H. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Analytical Analysis

Standards such as but not limited to sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-Ill, LNFP-V, LNFP-VI, LSTa, LSTc and LSTd were purchased from Carbosynth (UK), Elicityl (France) and IsoSep (Sweden). Other compounds were analysed with in-house made standards.

Sialylated oligosaccharides were analysed on a Waters Acquity H-class UPLC with Refractive Index (RI) detection. A volume of 0. 5 μL sample was injected on a Waters Acquity UPLC BEH Amide column (2.1×100 mm;130 A;1.7 am). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C. Neutral oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm;130 A;1.7 μm) column with an Acquity UPLC BEH Amide VanGuard column, 130 A, 2.1×5 mm. The column temperature was 50° C. The mobile phase consisted of a % water and % acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C. Both neutral and sialylated sugars were analysed on a Waters Acquity H-class UPLC with Refractive Index (RI) detection. A volume of 0.5 μL sample was injected on a Waters Acquity UPLC BEH Amide column (2.1×100 mm;130 A;1.7 am). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray lonisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2 % of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 5. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

To produce sialic acid and CMP-sialic acid, a yeast expression plasmid can be derived from the pRS420-plasmid series (Christianson et al., 1992, Gene 110: 119-122) containing the TRP1 selection marker and constitutive transcriptional units for an L-glutamine-D-fructose-6-phosphate aminotransferase like e.g. the mutant glmS*54 from E. coli with SEQ ID NO 36, a phosphatase like any one or more of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225, an N-acetylglucosamine 2-epimerase like e.g. AGE from B. ovatus with SEQ ID NO 35, an N-acetylneuraminate (Neu(n)Ac) synthase chosen from the list comprising SEQ ID NO 02, 03, 04, 05, 06, 07, 08, 09, 10, 11, 12, 13, 14, 15, 16, 17 and 18 and an N-acylneuraminate cytidylyltransferase like e.g. NeuA from P. multocida with SEQ ID NO 40. Optionally, a constitutive transcriptional unit for a glucosamine 6-phosphate N-acetyltransferase like e.g. GNA1 from S. cerevisiae with SEQ ID NO 34 was added as well. To produce sialylated oligosaccharides, the plasmid further comprised constitutive transcriptional units for a lactose permease like LAC12 from Kluyveromyces lactis with SEQ ID NO 72, and one or more sialyltransferases like e.g. SEQ ID NOs 41 to 45 or 80.

To produce GDP-fucose, a yeast expression plasmid like p2a_2p_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2p yeast on and the Ura3 selection marker for selection and maintenance in yeast. This plasmid is further modified with constitutive transcriptional units for a lactose permease like e.g. LAC12 from K. lactis with SEQ ID NO 72, a GDP-mannose 4,6-dehydratase like e.g. gmd from E. coli with SEQ ID NO 56 and a GDP-L-fucose synthase like e.g. fcl from E. coli with SEQ ID NO 57. The yeast expression plasmid p2a_2μ_Fuc2 can be used as an alternative expression plasmid of the p2a_2μ_Fuc plasmid comprising next to the ampicillin resistance gene, the bacterial ori, the 2μ yeast on and the Ura3 selection marker constitutive transcriptional units for a lactose permease like e.g. LAC12 from K. lactis with SEQ ID NO 72, a fucose permease like e.g. fucP from E. coli with SEQ ID NO 58 and a bifunctional fucose kinase/fucose-1-phosphate guanylyltransferase like e.g. fkp from B. fragilis with SEQ NO ID 59. To further produce fucosylated oligosaccharides, the p2a_2μ_Fuc and its variant the p2a_2μ_Fuc2, additionally contained (a) constitutive transcriptional unit(s) for one or more fucosyltransferases like e.g. SEQ ID NOs 50 and 51.

To produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g. galE from E. coli with SEQ ID NO 63. This plasmid was further modified with constitutive transcriptional units for a lactose permease like e.g. LAC12 from K. lactis with SEQ ID NO 72, a galactoside beta-1,3-N-acetylglucosaminyltransferase like e.g. IgtA from N. meningitidis with SEQ ID NO 60 to produce LN3. To further produce LN3-derived oligosaccharides like LNT or LNnT, an N-acetylglucosamine beta-1,3-galactosyltransferase like e.g. WbgO from E. coli 055:H7 with SEQ ID NO 61 or an N-acetylglucosamine beta-1,4-galactosyltransferase like e.g. IgtB from N. meningitidis with SEQ ID NO 62, respectively, was also added on the plasmid.

Preferably but not necessarily, the glycosyltransferases were N-terminally fused to a SUMOstar tag (e.g. obtained from pYSUMOstar, Life Sensors, Malvern, PA) to enhance their solubility.

Plasmids were maintained in the host E. coli DH5alpha (F-, phi80dlacZdeltaMl5, delta(lacZYA-argF)U169, deoR, recA1, endA1, hsdR17(rk−, mk+), phoA, supE44, lambda-, thi-1, gyrA96, re/A1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by Blazeck (Biotechnology and Bioengineering, Vol. 109, No. 11, 2012).

### Example 6. Production of Sialic Acid (Neu5Ac) with a Modified E. coli Strain Expressing a Neu(n)Ac Synthase with SEQ ID NO 02

An E. coli K-12 strain MG1655 was modified for sialic acid production as described in Example 4 comprising knock-outs of the E. coli nagA, nagB, nanA, nanE, nanK, LacZ and LacA genes and genomic knock-ins of constitutive transcriptional units containing the L-glutamine-D-fructose-6-phosphate aminotransferase glmS*54 from E. coli with SEQ ID NO 36, the glucosamine 6-phosphate N-acetyltransferase GNA1 from S. cerevisiae with SEQ ID NO 34, the N-acetylglucosamine 2-epimerase AGE from B. ovatus with SEQ ID NO 35, the N-acetylneuraminate (Neu(n)Ac) synthase from Paeniclostridium sordellii UMC1 with SEQ ID NO 02, the sucrose transporter (CscB) from E. coli W with SEQ ID NO 47, the fructose kinase (Frk) from Z. mobilis with SEQ ID NO 48 and the sucrose phosphorylase from B. adolescentis with SEQ ID NO 49. The novel strain was evaluated in a growth experiment according to the culture conditions provided in Example 4, in which the culture medium contained sucrose. The strain was grown in four biological replicates in α 96-well plate. After 72h of incubation, the culture broth was harvested, and the sugars were analysed on UPLC. The experiment demonstrated the novel strain produces sialic acid (Neu5Ac) in whole broth samples.

### Example 7. Production of 6′-Sialyllactose (6′-SL) with a Modified E. coli Strain Expressing a Neu(n)Ac Synthase with SEQ ID NO 02

An E. coli K-12 strain MG1655 modified for sialic acid production as described in Example 6 was further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,6-sialyltransferase from P. damselae with SEQ ID NO 43. The mutant strain was further adapted with an expression plasmid comprising constitutive transcriptional units for an N-acylneuraminate cytidylyltransferase (NeuA) from P. multocida with SEQ ID NO 40 and a beta-galactoside alpha-2,6-sialyltransferase from P. damselae with SEQ ID NO 43. The novel strain was evaluated in a growth experiment according to the culture conditions provided in Example 4, in which the culture medium contained sucrose and lactose. The strain was grown in four biological replicates in α 96-well plate. After 72h of incubation, the culture broth was harvested, and the sugars were analysed on UPLC. The experiment demonstrated the novel strain produced 0.19±0.08 g/L sialic acid (Neu5Ac) and 2.43±0.31 g/L 6′-sialyllactose in whole broth samples.

### Example 8. Production of 6′-Sialyllactose (6′-SL) with a Modified E. coli Strain Expressing a Neu(n)Ac Synthase with SEQ ID NO 02

An E. coli K-12 strain MG1655 modified for sialic acid production as described in Example 6 was further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,6-sialyltransferase from Photobacterium sp. JT-ISH-224 with SEQ ID NO 44. The mutant strain was further adapted with an expression plasmid comprising constitutive transcriptional units for an N-acylneuraminate cytidylyltransferase (NeuA) from P. multocida with SEQ ID NO 40 and a beta-galactoside alpha-2,6-sialyltransferase from Photobacterium sp. JT-ISH-224 with SEQ ID NO 44. The novel strain was evaluated in a growth experiment according to the culture conditions provided in Example 4, in which the culture medium contained sucrose and lactose. The strain was grown in four biological replicates in α 96-well plate. After 72h of incubation, the culture broth was harvested, and the sugars were analysed on UPLC. The experiment demonstrated the novel strain produces 6′-sialyllactose in whole broth samples.

### Example 9. Production of 3′-Sialyllactose (3′-SL) with a Modified E. coli Strain Expressing a Neu(n)Ac Synthase with SEQ ID NO 02

An E. coli K-12 strain MG1655 modified for sialic acid production as described in Example 6 is further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,3-sialyltransferase from P. multocida subsp. multocida str. Pm70 with SEQ ID NO 41. The mutant strain is further adapted with an expression plasmid comprising constitutive transcriptional units for an N-acylneuraminate cytidylyltransferase (NeuA) from P. multocida with SEQ ID NO 40 and a beta-galactoside alpha-2,3-sialyltransferase from P. multocida subsp. multocida str. Pm70 with SEQ ID NO 41. The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 10. Production of 3′-Sialyllactose (3′-SL) with a Modified E. coli Strain Expressing a Neu(n)Ac Synthase with SEQ ID NO 02

An E. coli K-12 strain MG1655 modified for sialic acid production as described in Example 6 was further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,3-sialyltransferase from N. meningitidis with SEQ ID NO 42. The mutant strain was further adapted with an expression plasmid comprising constitutive transcriptional units for an N-acylneuraminate cytidylyltransferase (NeuA) from P. multocida with SEQ ID NO 40 and a beta-galactoside alpha-2,3-sialyltransferase from N. meningitidis with SEQ ID NO 42. The novel strain was evaluated in a growth experiment according to the culture conditions provided in Example 4, in which the culture medium contained sucrose and lactose. The strain was grown in four biological replicates in α 96-well plate. After 72h of incubation, the culture broth was harvested, and the sugars were analysed on UPLC. The experiment demonstrated the novel strain produces 3′-sialyllactose in whole broth samples.

### Example 11. Production of 3′-Sialyllactose (3′-SL) with a Modified E. coli Strain Expressing a Neu(n)Ac Synthase with SEQ ID NO 02

An E. coli K-12 strain MG1655 modified for sialic acid production as described in Example 6 is further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferase from C. jejuni with SEQ ID NO 64, and the beta-galactoside alpha-2,3-sialyltransferase from P. multocida with SEQ ID NO 80. The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 12. Production of 3′-Sialyllactose (3′-SL) with a Modified E. coli Strain Expressing a Neu(n)Ac Synthase with SEQ ID NO 02

An E. coli K-12 strain MG1655 modified for sialic acid production as described in Example 6 is further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,3-sialyltransferase from P. multocida with SEQ ID NO 80. The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 13. Production of 3′-Sialyllactose (3′-SL) with a Modified E. coli Strain Expressing a Neu(n)Ac Synthase with SEQ ID NO 02

An E. coli K-12 strain MG1655 modified for sialic acid production as described in Example 6 is further modified with a knock-out of the E. coli LacY and nanT genes and genomic knock-ins of constitutive transcriptional units containing the lactose permease (LacY) from E. coli with SEQ ID NO 46, the sialic acid transporter (nanT) from E. coli with SEQ ID NO 66, the N-acylneuraminate cytidylyltransferases from C. jejuni with SEQ ID NO 64 and from H. influenzae with SEQ ID NO 65, and the beta-galactoside alpha-2,3-sialyltransferase from P. multocida with SEQ ID NO 80. The mutant strain is further adapted with an expression plasmid comprising constitutive transcriptional units for an N-acylneuraminate cytidylyltransferase (NeuA) from P. multocida with SEQ ID NO 40 and a beta-galactoside alpha-2,3-sialyltransferase from P. multocida with SEQ ID NO 80. The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 14. Evaluation of Mutant E. coli Strains for Production of Sialic Acid (Neu5Ac) with Different Neu(n)Ac synthases

In the next example, different Neu(n)Ac synthases were evaluated in mutant E. coli strains for production of sialic acid. These Neu(n)Ac synthases were chosen from Aeromonas caviae (SEQ ID NO 67), Candidatus koribacter versatilis (SEQ ID NO 68), Legionella pneumophila (SEQ ID NO 69), Methanocaldococcus jannaschii (SEQ ID NO 70) and Moritella viscosa (SEQ ID NO 71) and are annotated in UniProt (www.uniprot.org) on 23 Feb. 2021 to be a neuB (SEQ ID NO 67), an N-acetylneuraminate synthase (SEQ ID NO 68), an N-acetylneuraminic acid condensing enzyme (SEQ ID NO 69), an N-acetylneuraminic acid synthase (SEQ ID NO 71) or to have N-acetylneuraminate-9-phosphate synthase activity (SEQ ID NO 70). The Neu(n)Ac synthase from Paeniclostridium sordellii UMC1 with SEQ ID NO 02 and having the sequence [ILMV][MST]XXXXX(X, no K)(X, no Q)XXXXXXXXXXXX(X, no I)XXX(X, no T)X[ACS][ST][AP][FWY]XXX(X, no G)X(X, no L)X[IL](X, no K)XXXX(X, no E)XXKXXS with SEQ ID NO 01, wherein X is any amino acid, was used as a reference Neu(n)Ac synthase and was proven in Examples 6 and 7 to be a functional Neu(n)Ac synthase involved in the synthesis of sialic acid (Neu5Ac).

An E. coli K-12 strain MG1655 was modified for sialic acid production as described in Example 4 comprising knock-outs of the E. coli nagA, nagB, nanA, nanT, nanE, nanK, LacZ and LacA genes and genomic knock-ins of constitutive transcriptional units containing the sialic acid transporter (nanT) from E. coli with SEQ ID NO 66, the L-glutamine-D-fructose-6-phosphate aminotransferase glmS*54 from E. coli with SEQ ID NO 36, the glucosamine 6-phosphate N-acetyltransferase GNA1 from S. cerevisiae with SEQ ID NO 34, the N-acetylglucosamine 2-epimerase AGE from B. ovatus with SEQ ID NO 35, an N-acetylneuraminate (Neu(n)Ac) synthase chosen from SEQ ID NO 02, 67, 68, 69, 70 or 71, the sucrose transporter (CscB) from E. coli W with SEQ ID NO 47, the fructose kinase (Frk) from Z. mobilis with SEQ ID NO 48 and the sucrose phosphorylase from B. adolescentis with SEQ ID NO 49. The novel strains were evaluated in a growth experiment according to the culture conditions provided in Example 4, in which the culture medium contained sucrose. Each strain was grown in four biological replicates in α 96-well plate. After 72h of incubation, the culture broth was harvested, and the sugars were analysed on UPLC. The experiment demonstrated that the E. coli strains expressing a Neu(n)Ac synthase chosen from Aeromonas caviae (SEQ ID NO 67), Candidatus koribacter versatilis (SEQ ID NO 68), Legionella pneumophila (SEQ ID NO 69), Methanocaldococcus jannaschii (SEQ ID NO 70) or Moritella viscosa (SEQ ID NO 71) did not produce sialic acid (Neu5Ac) in whole broth samples. In contrast, the reference strain expressing the Neu(n)Ac synthase chosen from Paeniclostridium sordellii UMC1 with SEQ ID NO 02 produced sialic acid (Neu5Ac) in whole broth samples.

### Example 15. Production of Sialic Acid and 6′-Sialyllactose with a Modified E. coli Strain in Fed-Batch Fermentations

The mutant E. coli strain as described in Example 7 was further evaluated in a fed-batch fermentation process. Fed-batch fermentations at bioreactor scale were performed as described in Example 4. In these examples, sucrose was used as a carbon source and lactose was added in the batch medium as a precursor. No sialic acid (Neu5Ac) was added to the fermentation process. In contrast to the cultivation experiments that are described herein and wherein only end samples were taken at the end of cultivation (i.e. 72 hours as described herein), regular broth samples were taken at several time points during the fermentation process and the production of sialic acid (Neu5Ac) and 6′-sialyllactose at each of said time points was measured using UPLC as described in Example 4. The experiment demonstrated that broth samples taken e.g. at the end of the batch phase and during fed-batch phase comprised sialic acid production together with 6′-sialyllactose and unmodified lactose. Broth samples taken at the end of the fed-batch phase comprised 6′-sialyllactose and almost no or a very low concentration of Neu5Ac and almost no or a very low concentration of unmodified lactose demonstrating almost all or all of the precursor lactose was modified with almost all or all Neu5Ac produced during the fermentation of the mutant cells producing 6′-SL.

### Example 16. Production of Sialic Acid and 3′-Sialyllactose with a Modified E. coli Strain in Fed-Batch Fermentations

The mutant E. coli strains as described in Examples 9 to 13 are further evaluated in a fed-batch fermentation process. Fed-batch fermentations at bioreactor scale are performed as described in Example 4. In these examples, sucrose is used as a carbon source and lactose is added in the batch medium as a precursor. No sialic acid (Neu5Ac) is added to the fermentation process. In contrast to the cultivation experiments that are described herein and wherein only end samples were taken at the end of cultivation (i.e. 72 hours as described herein), regular broth samples are taken at several time points during the fermentation process and the production of sialic acid (Neu5Ac) and 3′-sialyllactose at each of said time points is measured using UPLC as described in Example 4.

### Example 17. Production of an Oligosaccharide Mixture Comprising 6′SL, LN3, Sialylated LN3, LNnT and LSTc with a Modified E. coli Host

E. coli hosts modified for 6′-siayllactose as described in Examples 7 and 8 are further modified with genomic knock-ins comprising constitutive transcriptional units of the galactoside beta-1,3-N-acetylglucosaminyltransferase LgtA from N. meningitidis with SEQ ID NO 60 for production of LN3 and of the N-acetylglucosamine beta-1,4-galactosyltransferase LgtB from N. meningitidis with SEQ ID NO 62 to produce LNnT, as described in Example 4, to produce a mixture of oligosaccharides comprising 6′SL, LN3, LNnT and LSTc (Neu5Ac-a2,6-Gal-b1,4-GIcNAc-b1,3-Gal-b1,4-Glc). The novel strains are evaluated in a growth experiment according to the culture conditions provided in Example 4, in which the culture medium contains sucrose and lactose. Each strain is grown in four biological replicates in α 96-well plate. After 72h of incubation, the culture broth is harvested, and the sugars are analysed on UPLC.

### Example 18. Production of an Oligosaccharide Mixture Comprising LN3, Sialylated LN3, LNT, 3′-SL and LSTa with a Modified E. coli Host

An E. coli host modified for sialic acid production (Neu5Ac) and 3′-siayllactose as described in Example 13 is further modified with genomic knock-ins comprising constitutive transcriptional units of the galactoside beta-1,3-N-acetylglucosaminyltransferase LgtA from N. meningitidis with SEQ ID NO 60 for production of LN3 and of the N-acetylglucosamine beta-1,3-galactosyltransferase WbgO from E. coli 055:H7 with SEQ ID NO 61 to produce LNT, as described in Example 4, to produce a mixture of oligosaccharides comprising LN3, 3′-sialylated LN3 (Neu5Ac-a2,3-GIcNAc-b1,3-Gal-b1,4-Glc), LNT, 3′SL and LSTa (Neu5Ac-a2,3-Gal-b1,3-GIcNAc-b1,3-Gal-b1,4-Glc). The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 19. Production of an Oligosaccharide Mixture Comprising LN3, Sialylated LN3, LNnT, 3′-SL and LSTd with a Modified E. coli Host

An E. coli host modified for sialic acid production (Neu5Ac) and 3′-siayllactose as described in Example 13 is further modified with genomic knock-ins comprising constitutive transcriptional units of the galactoside beta-1,3-N-acetylglucosaminyltransferase LgtA from N. meningitidis with SEQ ID NO 60 for production of LN3 and of the N-acetylglucosamine beta-1,4-galactosyltransferase LgtB from N. meningitidis with SEQ ID NO 62 to produce LNnT, as described in Example 4, to produce a mixture of oligosaccharides comprising 3′SL, LN3, 3′-sialylated LN3 (Neu5Ac-a2,3-GIcNAc-b1,3-Gal-b1,4-Glc), LNnT and LSTd (Neu5Ac-a2,3-Gal-b1,4-GlcNAc-b1,3-Gal-b1,4-Glc). The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 20. Production of Sialic Acid (Neu5Ac) with a Modified E. coli Strain Expressing a Neu(n)Ac Synthase with SEQ ID NO 02

An E. coli K-12 strain MG1655 is modified for sialic acid production as described in Example 4 comprising knock-outs of the E. coli nagA, nagB, nanA, nanE, nanK, LacZ and LacA genes and genomic knock-ins of constitutive transcriptional units containing the L-glutamine-D-fructose-6-phosphate aminotransferase glmS*54 from E. coli with SEQ ID NO 36, the phosphoglucosamine mutase glmM from E. coli with SEQ ID NO 37, the N-acetylglucosamine-1-phosphate uridyltransferase/glucosamine-1-phosphate acetyltransferase glmU from E. coli with SEQ ID NO 38, the UDP-N-acetylglucosamine 2-epimerase NeuC from Campylobacter jejuni with SEQ ID NO 39, the N-acetylneuraminate (Neu(n)Ac) synthase from Paeniclostridium sordellii UMC1 with SEQ ID NO 02, the sucrose transporter CscB from E. coli W with SEQ ID NO 47, the fructose kinase Frk from Z. mobilis with SEQ ID NO 48 and the sucrose phosphorylase from B. adolescentis with SEQ ID NO 49. The novel strain is evaluated in a growth experiment according to the culture conditions provided in Example 4, in which the culture medium contains sucrose. The strain is grown in four biological replicates in α 96-well plate. After 72h of incubation, the culture broth is harvested, and the sugars are analysed on UPLC.

### Example 21. Production of Sialic Acid (Neu5Ac) and 6′-Sialyllactose (6′-SL) with a Modified E. Coli Strain Expressing a Neu(n)Ac Synthase with SEQ ID NO 02

A mutant E. coli K-12 strain MG1655 producing sialic acid as described in Example 20 is further modified with knock-outs of the LacZ and nanT genes and genomic knock-ins of constitutive transcriptional units containing the lactose permease (LacY) from E. coli with SEQ ID NO 46, the sialic acid transporter (nanT) from E. coli with SEQ ID NO 66 and the N-acylneuraminate cytidylyltransferase NeuA from C. jejuni with SEQ ID NO 64. The mutant strain is further adapted with an expression plasmid comprising constitutive transcriptional units for the N-acylneuraminate cytidylyltransferase NeuA from P. multocida with SEQ ID NO 40 and the beta-galactoside alpha-2,6-sialyltransferase from P. damselae with SEQ ID NO 43. The novel strain is evaluated in a growth experiment according to the culture conditions provided in Example 4, in which the culture medium contains sucrose and lactose. The strain is grown in four biological replicates in α 96-well plate. After 72h of incubation, the culture broth is harvested, and the sugars are analysed on UPLC.

### Example 22. Production of Sialic Acid (Neu5Ac) with a Modified E. coli Strain Expressing an N-Acylneuraminate-9-Phosphate Synthetase and a Phosphatase

An E. coli K-12 strain MG1655 is modified for sialic acid production as described in Example 4 comprising knock-outs of the E. coli nagA, nagB, nanA, nanE, nanK, LacZ and LacA genes and genomic knock-ins of constitutive transcriptional units containing the L-glutamine-D-fructose-6-phosphate aminotransferase glmS*54 from E. coli with SEQ ID NO 36, the phosphoglucosamine mutase glmM from E. coli with SEQ ID NO 37, the N-acetylglucosamine-1-phosphate uridyltransferase/glucosamine-1-phosphate acetyltransferase glmU from E. coli with SEQ ID NO 38, the bifunctional UDP-GlcNAc 2-epimerase/N-acetylmannosamine kinase from Mus musculus (strain C57BL/6J) with SEQ ID NO 33, an N-acylneuraminate-9-phosphate synthetase chosen from the list comprising SEQ ID NO 21, 22, 23, 24, 25, 26, 27 and 28, and an N-acylneuraminate-9-phosphatase from Candidatus Magnetomorum sp. HK-1 with SEQ ID NO 30. The novel strains are evaluated in a growth experiment according to the culture conditions provided in Example 4, in which the culture medium contains glucose. Each strain is grown in four biological replicates in α 96-well plate. After 72h of incubation, the culture broth is harvested, and the sugars are analysed on UPLC.

### Example 23. Production of Sialic Acid (Neu5Ac) with a Modified E. coli Strain Expressing an N-Acylneuraminate-9-Phosphate Synthetase and a Phosphatase

An E. coli K-12 strain MG1655 is modified for sialic acid production as described in Example 4 comprising knock-outs of the E. coli nagA, nagB, nanA, nanE, nanK, LacZ and LacA genes and genomic knock-ins of constitutive transcriptional units containing the L-glutamine-D-fructose-6-phosphate aminotransferase glmS*54 from E. coli with SEQ ID NO 36, the phosphoglucosamine mutase glmM from E. coli with SEQ ID NO 37, the N-acetylglucosamine-1-phosphate uridyltransferase/glucosamine-1-phosphate acetyltransferase glmU from E. coli with SEQ ID NO 38, the bifunctional UDP-GlcNAc 2-epimerase/N-acetylmannosamine kinase from Mus musculus (strain C57BL/6J) with SEQ ID NO 33, an N-acylneuraminate-9-phosphate synthetase chosen from the list comprising SEQ ID NO 21, 22, 23, 24, 25, 26, 27 and 28, and an N-acylneuraminate-9-phosphatase from Bacteroides thetaiotaomicron (strain ATCC 29148) with SEQ ID NO 32. The novel strains are evaluated in a growth experiment according to the culture conditions provided in Example 4, in which the culture medium contains glucose. Each strain is grown in four biological replicates in α 96-well plate. After 72h of incubation, the culture broth is harvested, and the sugars are analysed on UPLC.

### Example 24. Production of 6′-Sialyllactose (6′-SL) with a Modified E. coli Strain Expressing an N-Acylneuraminate-9-Phosphate Synthetase and a Phosphatase

The mutant E. coli strains modified for Neu5Ac production as described in Examples 22 and 23 are further modified with and genomic knock-ins of constitutive transcriptional units containing the lactose permease (LacY) from E. coli with SEQ ID NO 46 and the N-acylneuraminate cytidylyltransferase (neuA) from C. jejuni with SEQ ID NO 64. The mutant strains are further adapted with an expression plasmid comprising constitutive transcriptional units for an N-acylneuraminate cytidylyltransferase (NeuA) from P. multocida with SEQ ID NO 40 and a beta-galactoside alpha-2,6-sialyltransferase from P. damselae with SEQ ID NO 43. The novel strains are evaluated in a growth experiment according to the culture conditions provided in Example 4, in which the culture medium contains glucose and lactose. Each strain is grown in four biological replicates in α 96-well plate. After 72h of incubation, the culture broth is harvested, and the sugars are analysed on UPLC.

### Example 25. Production of 3′-Sialyllactose (3′-SL) with a Modified E. coli Strain Expressing an N-Acylneuraminate-9-Phosphate Synthetase and a Phosphatase

The mutant E. coli strains modified for Neu5Ac production as described in Examples 22 and 23 are further modified with and genomic knock-ins of constitutive transcriptional units containing the lactose permease (LacY) from E. coli with SEQ ID NO 46 and the N-acylneuraminate cytidylyltransferase (neuA) from C. jejuni with SEQ ID NO 64. The mutant strains are further adapted with an expression plasmid comprising constitutive transcriptional units for an N-acylneuraminate cytidylyltransferase (NeuA) from P. multocida with SEQ ID NO 40 and a beta-galactoside alpha-2,3-sialyltransferase from P. multocida with SEQ ID NO 80. The novel strains are evaluated in a growth experiment according to the culture conditions provided in Example 4, in which the culture medium contains glucose and lactose. Each strain is grown in four biological replicates in α 96-well plate. After 72h of incubation, the culture broth is harvested, and the sugars are analysed on UPLC.

### Example 26. Production of Sialic Acid (Neu5Ac) with a Modified S. cerevisiae Strain Expressing a Neu(n)Ac Synthase with SEQ ID NO 02

A S. cerevisiae strain is adapted for sialic acid (Neu5Ac) production as described in Example 5 with a pRS420-derived yeast expression plasmid comprising the TRP1 selection marker and constitutive transcriptional units for the mutant glmS*54 from E. coli with SEQ ID NO 36, a phosphatase like any one or more of e.g. the E. coligenes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO18122225, the N-acetylglucosamine 2-epimerase AGE from B. ovatus with SEQ ID NO 35 and the Neu(n)Ac synthase from Paeniclostridium sordellii UMC1 with SEQ ID NO 02. The novel strain is evaluated in a growth experiment on SD CSM-Trp drop-out medium according to the culture conditions provided in Example 5. After 72h of incubation, the culture broth is harvested, and the sugars are analysed on UPLC.

### Example 27. Production of 6′-Sialyllactose (6′-SL) with a Modified S. cerevisiae Strain Expressing a Neu(n)Ac Synthase with SEQ ID NO 02

A S. cerevisiae strain is adapted for sialic acid (Neu5Ac) and 6′-sialyllactose production as described in Example 5 with a pRS420-derived yeast expression plasmid comprising the TRP1 selection marker and constitutive transcriptional units for the mutant glmS*54 from E. coli with SEQ ID NO 36, a phosphatase like any one or more of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO18122225, the N-acetylglucosamine 2-epimerase AGE from B. ovatus with SEQ ID NO 35, the Neu(n)Ac synthase from Paeniclostridium sordellii UMC1 with SEQ ID NO 02, the N-acylneuraminate cytidylyltransferase NeuA from P. multocida with SEQ ID NO 40, the beta-galactoside alpha-2,6-sialyltransferase from P. damselae with SEQ ID NO 43 and the lactose permease LAC12 from K. lactis with SEQ ID NO 72. The novel strain is evaluated in a growth experiment on SD CSM-Trp drop-out medium comprising lactose as precursor according to the culture conditions provided in Example 5. After 72h of incubation, the culture broth is harvested, and the sugars are analysed on UPLC.

### Example 28. Production of an Oligosaccharide Mixture Comprising 6′-SL, LN3, Sialylated LN3, LNnT and LSTc with a Modified S. cerevisiae Host

The mutant S. cerevisiae strain described in Example 27 is further modified with a second pRS420-derived yeast expression plasmid comprising the HIS3 selection marker and constitutive transcriptional units for galE from E. coli with SEQ ID NO 63, the galactoside beta-1,3-N-acetylglucosaminyltransferase (IgtA) from N. meningitidis with SEQ ID NO 60 and the N-acetylglucosamine beta-1,4-galactosyltransferase (IgtB) from N. meningitidis with SEQ ID NO 62 to produce a mixture of oligosaccharides comprising 6′-SL, LN3, LNnT and LSTc (Neu5Ac-a2,6-Gal-b1,4-GIcNAc-b1,3-Gal-b1,4-Glc). The novel strain is evaluated in a growth experiment on SD CSM-Trp-His drop-out medium comprising lactose as precursor according to the culture conditions provided in Example 5. After 72h of incubation, the culture broth is harvested, and the sugars are analysed on UPLC.

### Example 29. Production of 3′-Sialyllactose (3′-SL) with a Modified S. cerevisiae Strain Expressing a Neu(n)Ac Synthase with SEQ ID NO 02

A S. cerevisiae strain is adapted for sialic acid (Neu5Ac) and 3′-sialyllactose production as described in Example 5 with a pRS420-derived yeast expression plasmid comprising the TRP1 selection marker and constitutive transcriptional units for the mutant glmS*54 from E. coli with SEQ ID NO 36, a phosphatase like any one or more of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO18122225, the N-acetylglucosamine 2-epimerase AGE from B. ovatus with SEQ ID NO 35, the Neu(n)Ac synthase from Paeniclostridium sordellii UMC1 with SEQ ID NO 02, the N-acylneuraminate cytidylyltransferase NeuA from P. multocida with SEQ ID NO 40, the beta-galactoside alpha-2,3-sialyltransferase from P. multocida with SEQ ID NO 80 and the lactose permease LAC12 from K. lactis with SEQ ID NO 72. The novel strain is evaluated in a growth experiment on SD CSM-Trp drop-out medium comprising lactose as precursor according to the culture conditions provided in Example 5. After 72h of incubation, the culture broth is harvested, and the sugars are analysed on UPLC.

### Example 30. Production of an Oligosaccharide Mixture Comprising LN3, Sialylated LN3, LNT, 3′-SL and LSTa with a modified S. cerevisiae host

The mutant S. cerevisiae strain described in Example 29 is further modified with a second pRS420-derived yeast expression plasmid comprising the HIS3 selection marker and constitutive transcriptional units for the UDP-glucose-4-epimerase galE from E. coli with SEQ ID NO 63, the galactoside beta-1,3-N-acetylglucosaminyltransferase IgtA from N. meningitidis with SEQ ID NO 60 and the N-acetylglucosamine beta-1,3-galactosyltransferase WbgO from E. coli 055:H7 with SEQ ID NO 61 to produce a mixture of oligosaccharides comprising LN3, 3′-sialylated LN3 (Neu5Ac-a2,3-GIcNAc-b1,3-Gal-b1,4-Glc), LNT, 3′SL and LSTa (Neu5Ac-a2,3-Gal-b1,3-GIcNAc-b1,3-Gal-b1,4-Glc). The novel strain is evaluated in a growth experiment on SD CSM-Trp-His drop-out medium comprising lactose as precursor according to the culture conditions provided in Example 5. After 72h of incubation, the culture broth is harvested, and the sugars are analysed on UPLC.

### Example 31. Production of an Oligosaccharide Mixture Comprising LN3, Sialylated LN3, LNnT, 3′-SL and LSTd with a Modified S. cerevisiae Host

The mutant S. cerevisiae strain described in Example 29 is further modified with a second pRS420-derived yeast expression plasmid comprising the HIS3 selection marker and constitutive transcriptional units for the UDP-glucose-4-epimerase galE from E. coli with SEQ ID NO 63, the galactoside beta-1,3-N-acetylglucosaminyltransferase IgtA from N. meningitidis with SEQ ID NO 60 and the N-acetylglucosamine beta-1,4-galactosyltransferase LgtB from N. meningitidis with SEQ ID NO 62 to produce a mixture of oligosaccharides comprising 3′SL, LN3, 3′-sialylated LN3 (Neu5Ac-a2,3-GIcNAc-b1,3-Gal-b1,4-Glc), LNnT and LSTd (Neu5Ac-a2,3-Gal-b1,4-GIcNAc-b1,3-Gal-b1,4-Glc). The novel strain is evaluated in a growth experiment on SD CSM-Trp-His drop-out medium comprising lactose as precursor according to the culture conditions provided in Example 5. After 72h of incubation, the culture broth is harvested, and the sugars are analysed on UPLC.

1-56. (canceled)

57. A metabolically engineered cell for producing a bioproduct selected from monosaccharide, activated monosaccharide, phosphorylated monosaccharide, disaccharide, oligosaccharide, aglycon, glycolipid and glycoprotein, the cell comprising:
a pathway for the bioproduct,
wherein the bioproduct is an N-acetylneuraminic acid (neu(n)Ac)-containing compound wherein (n) is 4, 5, 7, 8 or 9 or a combination thereof, and
wherein optionally the pathway has at least one glycosyltransferase that is involved in the production of the bioproduct.

58. The cell of claim 57, wherein the cell is modified or has been modified in expression or activity of at least one Neu(n)Ac synthase, which has Neu(n)Ac synthase activity and which has PFAM domain PF3102 and/or PatricDB global family domain PGF_69734 and which comprises the sequence [ILMV][MST]XXXXX(X, NO:K)(X, NO:Q)XXXXXXXXXXXX(X, NO:I)XXX(X, NO:T)X[ACS][ST][AP][FWY]XXX(X, NO:G)X(X, NO:L)X[IL](X, NO:K)XXXX(X, NO:E)XXKXXS with SEQ ID NO:1, wherein X is any amino acid.

59. The cell of claim 57, wherein the cell is modified or has been modified in expression or activity of at least one Neu(n)Ac synthase, which has Neu(n)Ac synthase activity, and which has PFAM domain PF3102 and/or PatricDB global family domain PGF_69734 and which
comprises the polypeptide of any one of SEQ ID NO:2, 3, 4, 5, 6, 7, 8, 9, 10, 11, 12, 13, 14, 15, 16, 17 or 18, or
is a functional homolog, variant, derivative or functional fragment, of any one of SEQ ID NO:2, 3, 4, 5, 6, 7, 8, 9, 10, 11, 12, 13, 14, 15, 16, 17 or 18 having at least 80% overall sequence identity to the full-length of any one of the polypeptide of SEQ ID NO:2, 3, 4, 5, 6, 7, 8, 9, 10, 11, 12, 13, 14, 15, 16, 17 or 18, respectively, and having Neu(n)Ac synthase activity.

60. The cell of claim 57, wherein the pathway for producing the Neu(n)Ac-containing compounds comprises at least one enzyme selected from the group consisting of N-acylglucosamine 2-epimerase, UDP-N-acetylglucosamine 2-epimerase, N-acetylmannosamine-6-phosphate 2-epimerase, bifunctional UDP-GlcNAc 2-epimerase/kinase, N-acylneuraminate-9-phosphate synthetase, phosphatase, CMP sialic acid synthase, and sialyltransferase.

61. The cell of claim 60, wherein N-acylneuraminate-9-phosphate synthetase is a polypeptide having N-acylneuraminate-9-phosphate synthetase activity and which has PFAM domain PF3102 and/or PatricDB global family domain PGF_69734 and which comprises the sequence [DE]XGXNHXGXXXXXXXMXXX[ACPS]XXXXXXXX[KR](Xa)[KR](Xb)[DE](Xc)KXXS (SEQ ID NO:19),
wherein X is any amino acid, a is 28 to 32, b is 28 to 30 and c is 14 to 16.

62. The cell of claim 61, wherein the polypeptide having N-acylneuraminate-9-phosphate synthetase activity comprises the sequence [DE]XGXNHXGXXXXXXXMXX(X, NO:I, L, M)[AS]XXXXXXXX[KR](Xa)[KR](Xb)[DE](Xc)KXXS (SEQ ID NO:20), wherein X is any amino acid, a is 28 to 32, b is 28 to 30 and c is 14 to 16.

63. The cell of claim 60, wherein N-acylneuraminate-9-phosphate synthetase is a polypeptide having N-acylneuraminate-9-phosphate synthetase activity and which has PFAM domain PF3102 and/or PatricDB global family domain PGF_69734 and which
comprises a polypeptide sequence according to any one of SEQ ID NO:21, 22, 23, 24, 25, 26, 27 or 28, or
is a functional homolog, variant, derivative or functional fragment of any one of SEQ ID NO:21, 22, 23, 24, 25, 26, 27 or 28 having at least 80% overall sequence identity to the full-length of any one of the polypeptide of SEQ ID NO:21, 22, 23, 24, 25, 26, 27 or 28, respectively, and having N-acylneuraminate-9-phosphate synthetase activity.

64. The cell of claim 60, wherein the phosphatase is a HAD-like phosphatase having N-acylneuraminate-9-phosphatase activity and:
comprising the sequence DXDGXXTDXXXXXXXXGXXXXXXXXXDXXXXXXXXXXXXXXX[ILV]X[ST]XXXXX XXXXRXXXL(Xa)K(Xb)GXDXXD(Xc)GXGXXR[DE] (SEQ ID NO:29), wherein X is any amino acid, a is 10 to 11, b is 21 and c is 32, or
comprising the sequence DXDXT[IL](Xa)TNGXXXXQXXK[IL](Xb)[KR]PXXX[IL][FWY](Xc)G[DN]XXXXD[ILV]X G (SEQ ID NO:31), wherein X is any amino acid, a is 110 to 160, b is 20 to 23 and c is 17.

65. The cell of claim 57, wherein the cell is modified for enhanced synthesis and/or supply of PEP.

66. The cell of claim 57, wherein the cell expresses at least one alpha-2,3-sialyltransferase, which has alpha-2,3-sialyltransferase activity and which
is the polypeptide of SEQ ID NO:80, or
is a polypeptide of 268 amino acid residues that is a functional homolog, variant, derivative or functional fragment of SEQ ID NO:80 having at least 80% overall sequence identity to the full-length of the polypeptide of SEQ ID NO:80 and having alpha-2,3-sialyltransferase activity.

67. The cell of claim 58, wherein the Neu(n)Ac synthase is an Neu5Ac synthase and wherein the Neu(n)Ac-containing compound is a sialylated compound comprising Neu5Ac.

68. The cell of claim 57, wherein the cell has a modification for reduced production of acetate.

69. The cell of claim 57, wherein the Neu(n)Ac-containing compound is selected from the group consisting of sialic acid, a disaccharide, an oligosaccharide, a glycolipid, and a glycoprotein.

70. The cell of claim 57, wherein the oligosaccharide is selected from the group consisting of a milk oligosaccharide, 0-antigen, an oligosaccharide repeat present in capsular polysaccharides, peptidoglycan, amino-sugars, and Lewis-type antigen oligosaccharide.

71. The cell of claim 57, wherein the cell is a bacterium, fungus, yeast, a plant cell, an animal cell, or a protozoan cell.

72. A method of producing a bioproduct by a cell, the method comprising the steps of:
(a) providing the cell of claim 57,
(b) cultivating the cell in a culture medium under conditions permissive to produce the bioproduct, and
(c) optionally separating the bioproduct from the cultivation.

73. The method according to claim 72, the method further comprising at least one of the following steps:
(d) adding to a culture medium in a reactor at least one precursor and/or acceptor feed wherein a total reactor volume ranges from 250 mL (milliliter) to 10.000 m³ (cubic meter); and
(e) adding at least one precursor and/or acceptor feed in a continuous manner to the culture medium over a course of at least 1 day by way of a feeding solution; and
the method resulting in a bioproduct, with a concentration of at least 50 g/L in a final volume of the culture medium.

74. The method according to claim 73, further comprising: at least one of the following steps:
(f) adding to the culture medium a lactose feed comprising at least 50 gram of lactose per liter of initial reactor volume wherein the reactor volume ranges from 250 mL to 10.000 m³ (cubic meter);
(g) adding a lactose feed in a continuous manner to the culture medium over the course of at least 1 day by way of a feeding solution; and
(h) adding a lactose feed in a continuous manner to the culture medium over the course of at least 1 day by way of a feeding solution and wherein the concentration of the lactose feeding solution is 50 g/L;
the method resulting in a bioproduct produced from the lactose with a concentration of at least 50 g/L.

75. The method according to claim 72, wherein separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography.

76. The method according to claim 72, wherein the method further comprises purifying the bioproduct.

77. The method according to claim 76, wherein the purification comprises at least one of the following: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying or lyophilization.


SEQUENCE LISTING (80 sequences)

SEQ ID NO: 1 - protein, 51 aa, Artificial sequence
  domain part 1
  VARIANT (1)..(1): Xaa can be Ile, Leu, Met or Val
  VARIANT (2)..(2): Xaa can be Met, Ser or Thr
  UNSURE (3)..(7): Xaa can be any naturally occurring amino acid
  UNSURE (8)..(8): Xaa can be any naturally occurring amino acid except Lys
  UNSURE (9)..(9): Xaa can be any naturally occurring amino acid except Gln
  UNSURE (10)..(21): Xaa can be any naturally occurring amino acid
  UNSURE (22)..(22): Xaa can be any naturally occurring amino acid except Ile
  UNSURE (23)..(25): Xaa can be any naturally occurring amino acid
  UNSURE (26)..(26): Xaa can be any naturally occurring amino acid except Thr
  UNSURE (27)..(27): Xaa can be any naturally occurring amino acid
  VARIANT (28)..(28): Xaa can be Ala, Cys or Ser
  VARIANT (29)..(29): Xaa can be Ser or Thr
  VARIANT (30)..(30): Xaa can be Ala or Pro
  VARIANT (31)..(31): Xaa can be Phe, Trp or Tyr
  UNSURE (32)..(34): Xaa can be any naturally occurring amino acid
  UNSURE (35)..(35): Xaa can be any naturally occurring amino acid except Gly
  UNSURE (36)..(36): Xaa can be any naturally occurring amino acid
  UNSURE (37)..(37): Xaa can be any naturally occurring amino acid except Leu
  UNSURE (38)..(38): Xaa can be any naturally occurring amino acid
  VARIANT (39)..(39): Xaa can be Ile or Leu
  UNSURE (40)..(40): Xaa can be any naturally occurring amino acid except Lys
  UNSURE (41)..(44): Xaa can be any naturally occurring amino acid
  UNSURE (45)..(45): Xaa can be any naturally occurring amino acid except Glu
  UNSURE (46)..(47): Xaa can be any naturally occurring amino acid
  UNSURE (49)..(50): Xaa can be any naturally occurring amino acid
XXXXXXXXXXXXXXXXXXXXXXXXXXXXXXXXXXXXXXXXXXXXXXXKXXS

SEQ ID NO: 2 - protein, 348 aa, Paeniclostridium sordellii UMC1
MNKNYMQIGNRKIGEDYAPLVIAEIGINHEGSLKTALEMVDAAYEAGAEVIKHQTHVVEDEMSKAAKKVIPGNTDVSIYDVMKRCALNEEDETKLKEYVESKGMIFISTPFSRSAANRLERMGVKAYKIGSGECNNYPLIDHIASFGKPIILSTGMNDIESIRKSVEIMEKHGVKYSLLHCTNLYPTPANLVRLGGMEELQKEFPNAIVGLSDHTVNNNACLAATALGASVLERHFTDSKDRPGPDIVCSMDPKELKELIEGSKEIQQMRGGKKVAAKEEQVTIDFAFATVVTTKDLKKGDVLSKDNIWVKRPGTGQIKAEHYESLIGKRINKDIENDEHLCWADIEE

SEQ ID NO: 3 - protein, 346 aa, Escherichia coli strain MOD1-EC5561
MEKVYIVAEIGCNHNGDFLIAKKMVSEAKMAGVDAVKFQTFKAELLISKYAPKAEYQKKVTDADESQLDMTKKLELPYDRYIELENYARSLGLDVFSTPFDLDSIDFLYSQKQKTWKIPSGELLNLPYIEKIAKLPIDDKRIIISTGMSTIDEIKLCLSVFDNNGVPKSNITILHCNTEYPTPFGDVNLSVISSYKKIFPEYNIGFSDHSPGFYAGIAAVPFGITFIEKHFTLDKTLPGPDHKASVTPDELRMLCEGIRCVELALGSPDKKVTDSEKKNKIVARKSIVAKTKIAKGDIFTVDNITTKRPGNGISPMYWYDILGKAAEFDFEEDELVVHTAYKPQEV

SEQ ID NO: 4 - protein, 346 aa, Providencia sp. wls1916
MNKIYIVAEIGCNHNGDFQLAKIMVDEAKAAGVDAVKFQTFKAELLISKIAPKAEYQIKVTGNTESQLEMTKKLELPYDQFIKLEKYAKELGLDVFSTPFDMDSIDFLATRNQKMWKIPSGELLNLPYLEKIAQLQIENKEIVLSTGMATIDEIKQALNILKGNGVHSNQITILHCNTEYPTPFEDVNLNAIKGLKNEFPEYRIGFSDHSSGYFAGIASVPYGITFIEKHFTLDKNFEGPDHKASVTPDELTLLCQGIRAVEKSLGSEEKKVTESERKNKIVARKSIVARVNIKKGDTYTVENITTKRPGNGISPMHWHEILGKTAESDFNEDQLIKHNGFDMQEV

SEQ ID NO: 5 - protein, 338 aa, Streptococcus ruminantium strain GUT187T
MVYIIAEIGCNHNGDVHLAKKMVDVAVQCGVDAVKFQTFKAEKLISKYAPKAEYQKETTGEKDSQLEMTRRLELSFDEYLELRDYCLSKHVDVFSTPFDEESLDFLISTNMPVYKIPSGEITNLPYLEKIGKQGKKVILSTGMAAIEEIHRAVGILRENGTTDISILHCTTEYPAPYRSLHLNVLTTLKKEFPNASIGYSDHSIGSEVPIAAVALGASIIEKHFTLDNAMEGPDHRASATPEVLSDLVKGVRIIEQALGRSEKIPDPVEEKNKIVARKSIVAKKDIKKGDIFSVDNITVKRPGDGISPMKWYDILGKVSEADFEVDQNIIHSDFANQL

SEQ ID NO: 6 - protein, 345 aa, Helicobacter mesocricetorum strain 87012_3
MQIQISHLKISEEVSPLVVPEIGINHNGSLELAKLMVDSAKRAGAKIIKHQTHIIEDEMSSEAKQVIPGNAKISIYEIMKKCALGYKDELALKEYAESKGLVYLSTPFSRAAANRLQDMGVSAFKIGSGECNNTPLLKHIASFKKPMILSTGMNNLASIKESVKILRDNEIPFVLLHTTNLYPTPPHLVRLGAMLELKKAFDCLVGLSDHTLDNLACLGATALGACVLERHFTDTMDREGPDIVCSMDEKALKELIIKSEQMAILRGLNASKMAASEEQVTIDFAFASVVSIQAITKGELLSKENIWVKRPGKGGIPASEFEKILGRRALRDISKDRQLSYEDFE

SEQ ID NO: 7 - protein, 349 aa, Campylobacter mucosalis strain ATCC 43264
MQNPKEFKIQNRLVGINHEPLIICEIGINHEGSIKTAFEMVDAAHRAGAEVIKHQTHIVEDEMSNEAKGVIPGNADVSIYEIMKRCALNEEDEIKLQKYVESKGMIFISTPFSRAAAHRLQKMNIPAYKIGSGECNNYPLVKLIASFGKPIILSTGMNDIQSIKKTVSIIESAGVPYALLHCTNVYPTKYSDVRLGAMSELMREFPNAVVGLSDHTTDNYACMGAVALGASILERHFTDSMHRVGPDIVCSMDPIAFKELKTASSALAKMRFGQKNTLIDDEKPTKAFAFASVVADADIKKGDVLSGENLWVKRPSGGDFGADEYESLFGKIAKNDIAKNSQIKKQDIY

SEQ ID NO: 8 - protein, 347 aa, Pseudoalteromonas luteoviolacea strain IPB1
MNPEFKIENRLVGIEHDPLVIAEIGINHEGSLKVAFEMVDAAIEAGAEIIKHQTHVVEDEMCGEAKKVIPGNADVSIYEIMERCALNEEDETKLKEYVESKGAIFISTPFSRAAAFRLERMGVSAYKIGSGECNNYPLVDLVASFGKPVILSTGMNDIPSIKKSVEIFEKYNTPYCLLHCTNVYPTPDHLIRLGGITELQEAFPNAVVGLSDHSVDNLACLSAVAVGASVLERHFTDCKSRPGPDIECSMDIKECADLIEQSKRVKAMRGGSKGAVEEEQVTIDFAYASVVTTQEIKQGELLTKDNLWVKRPGTGEFLAEDYEALLGKVAKVDIASDTMLASSHIEK

SEQ ID NO: 9 - protein, 348 aa, Ruminococcus albus strain AR67
MKSSFYIGDLKIGIGEEPLVIPEMGINHEGSLDVAMSIVDAAARAGAKVIKHQTHVVEDEMSHAAKKVIPGNTDVSIYEVMERCALSEDEEYELMRYVESKGMIFISTPFSRAAADRLHRFNVKAYKIGSGELNNYPLIKHIASFGKPMIVSTGMNNIESIRKAVNIMEDAGVPYALLHTTNLYPTVPNLVRLGAMQEMMETFPNIPIGLSDHTLTNTACISAITLGASIVERHFIDRKDRPGPDVICSMDEKECAELIIAARDVPQMLGGKKEAAKEEKVTIDFAFATVVTIKSVKAGEMFTKENLWVKRPGTGEILAEKYESILGKKSKVDIDNDTQLKWSMIDNI

SEQ ID NO: 10 - protein, 344 aa, Brevibacillus borstelensis cifa_chp40
MNELIIGDRKIGPEHPPLVIAEIGINHEGSFDKAIQMIDDAASVGCECVKFQTHVVDDEMIPNNVIPGNAKESIWEIMNRCALTKEEERELKKYTEAKGMIFLSTPFSRAAAVQLEELGVLAYKIGSGECNNYPLIEHIARYGKPVILSTGMNNLESIEKAVAILRKHNTPFALLHCTSIYPTPYDKVRLGALEELAQAFPDAVLGLSDHSLSNYPSLGAVALGARILERHFTSDKSWPGPDISISMDPTDLKELIEGSRAIFQALGGRKNVLKEEQPTIDFAYACVVAIRDIRAGEELSMENIWVKRPGTGEIKAEHFDSLLGKKALSPIKQNEQIRWAHVNG

SEQ ID NO: 11 - protein, 346 aa, Acidobacteria bacterium RIFCSPLOWO2_12_FULL_67_14b
MLPELSIGGRRVGPAHVPLVIAEIGINHEGSLDVARQIVDAAKRAGVEVVKHQTHVVDDEMSGAAKKVVPGNADVSIYQIMERCALSENDERRLKDYVESKGMIFMSTPFSRAAADRLERMNVPAYKIGSGECNNYPLLKHIASFGKPIILSTGMNTIESVGKAVAIFEQQGVPHALLHTTNLYPTPPHLVRLGAMVELGKAFPKAVIGLSDHTTDNLACLGAVALGASIVERHFIDTMSRVGPDIVCSMDEARCRELIQSAKILHAEIGGTKGPVKEEQVTMDFAFATVVTITPIKAGEALTKNNLWVKRPGTGSIPAERYDELLGKKARRDLPADTHLSLDDVE

SEQ ID NO: 12 - protein, 350 aa, Bacteroidetes bacterium GWA2_31_9b
MKTNPFFEIEGRKVGSEYDPLVIAELGINHNGSLQVAFEMVDAAHKAGVEIIKHQTHIVEDEMCKVAKNVIPGHTRESIYDIMQSCALSEEEEIELQRYVKSKGIIFISTPFSRAAANRLNKMKIPAYKIGSGECNNYPLIKHIATFGKPVILSTGMNSLETIKPAVEILRNNKIPFALLHCTNIYPTPPELIRLEAITQLKKEFPDAVVGLSDHSVTNYPCIASVALGASILERHFTDTMNRKGPDIICSMDQDTCKELIHATKIVRFARDGNKEPVDAEKPTIDFAFSCVVSIKPILAGEQFSYENIWVKRPGNGEIKAVHFESLIGKIASRAIENDTQLTWDDIKKH

SEQ ID NO: 13 - protein, 376 aa, Flavobacterium limicola
MQYSCPFESFGDCSRQAKLEDKIDNSQQKTMNPYIEIAGRKIGADYPPLVIAEIGINHEGSLAVAKEMVDAAKRAGVEVVKHQTHIVADEMSGAAKKVIPGNADVSIYEIMERCSLNEADELDLKKYVESKGMLFISTPFSRAAADRLENFDVPAYKIGSGECNNYPLLEHIASFGKPVLLSTGMNTIASIRKAIAIFDKHKVPVAVLHTTNLYPTPIRLVRFGAMMEMHHAFPDKVFGLSDHTLNNTACLGAVALGASILERHFTDHMQRTGPDIVCSMDEQTTKDLLLSSNEMWQMRGGVKEPALEEQVTIDFAFATVCAIKPIQKGDVLTKENIWVKRPGTGKILAEHFNDLIGKVATRTIANDEQLDWIDFA

SEQ ID NO: 14 - protein, 351 aa, uncultured Methylococcaceae bacterium strain Loc080925-8-8m_bin-121
MADFEIQGRRIGDDYPPVVIAEIGINHEGSLETAIEMADAAINAGAEIIKHQTHIIEDEMSDEAKLIIPGNADVSIYEIMKRCALSENDEYYLMKHVQKRGAIFISTPFSRAAADRLVRFNIPAYKIGSGECNNYPLVKHIARFGKPVILSTGMNSIESVRPSVEILRNARVPFALLHCTNVYPTPPELVRLGAMTKLKETFQDAVIGLSDHTTSNYACLGAVALGASILERHFTDRMDRPGPDIVCSMDPSALSTLIEGSKTIFKARGCDKKAVKAEAPTIAFAFASVVAIQDILPGQLLTEENIWVKRPGDGDFKALDFENLLGRVAVVPIRQGSQIKKSDINLTSNVR

SEQ ID NO: 15 - protein, 349 aa, Rhodobacteraceae bacterium HIMB11
MTHEATMKIGNREISPSHPPLVIAEIGINHGGDLEVAKNMVRLAALSGCECVKHQTHIVEDEMTDEAKQIFPPNADVSIWDVMAQCALSREDEAELKRYTEELGLIYISTPFSRAAADFLETLDVPAYKIGSGEADNLPLIRHIARKGKPVIMSTGMQTIETLQESVQILEDAGIEYALLECTNLYPSPPEIVSLQGVTDLKSAFPNAVVGFSDHSIGPEMALASVALGASILERHYTDTRYRKGPDIINSMDPAELRFLIDRSREIHTALMNPKQRTGPEEDVYRFARASVVADADLAAGHVITESDIWARRPGSGEIAGFEFDKVVGKTLTVAVTRNQQLKWSDFED

SEQ ID NO: 16 - protein, 348 aa, Arcobacter skirrowii strain L405
MHNKEIQIEKRKIGLNYPPLVIVEIGINHEGSLKTAFEMVDAAWRAGAEVIKHQTHVVEDEMSKAAKKVIPGNASVSIYEIMERCALNEEDEIKLKEYVESKGMIFISTPFSRAAADRLERMGVSSYKIGSGECNNYPLIEHIASFGKPMIVSTGMNDIESVRKTVNILEKYNVPYALLHTTNLYPTPVHLVRLGAMQELQKEFPNAIIGLSDHTTSNRACFAATALGACILERHFTDKMERSGPDIINSMDPIALQELIYGSAEIALMRGGKKEAAKEEQVTIDFAFATIVTIKPIKKGEQLTKENIWVKRPGTGPIKAEHYNELLGKTANKDIDFDEHIDWSDINE

SEQ ID NO: 17 - protein, 350 aa, Gelidibacter algens
MLQKQQPFIEIAGRKIGLDYPPLVIAEIGINHEGSLKTAKEMVDAAHRAGAEVVKHQTHIVSDEMSGAAKKVIPGNADISIYEIMERCALDETDELALKNYVESKGMIFISTPFSRAAAERLERMGVVAYKIGSGECNNYPLLEHIAQFGKPVILSTGMNTVESVKKAVAIFDAHHVPVALLHTTNLYPTPVHLVRFGAMIELHEAFPEKVFGLSDHTLNNNACLGAVALGASILERHFTDHMQRTGPDIVCSMDEQACKDLIHNSAEIAQMRGGSKNPAKEEQVTIDFAFATVCTIAPIKKGEYFTKDNIWVKRPGNGEFLAAHFNTILGKTATTDLDNDVQLRLKDVN

SEQ ID NO: 18 - protein, 345 aa, uncultured SAR86 cluster bacterium strain AG-909-A08
MNEFKLGNRMVGYHHPPLVIAEIGINHNGSLESAISLADDAIDAGIEVIKHQTHVVEDEMTSEAKSVIPGNSDKSIYQIMEECALNEEDEVSLKEHVESRGKIFISTPFSRAAAERLERMGVCGYKIGSGECNNYPLIEHIASFKKPIILSTGMNDLDSIRPAVEIIRKHQVPFALLHTTNIYPTPDRLVRLGAMNELQDTFSDAVIGLSDHTVSNHACFGAVALGASILERHFTDSMTREGPDIINSMDPSAAKELILGANTIFEQRGGSKGPVEEELPTIKFAFASIVSINEIKKGDVFSKENLWVKRPGTGEILAEEFESILGKKAKNDISSGQQLKREDIQ

SEQ ID NO: 19 - protein, 35 aa, Artificial sequence
  NANS domain
  VARIANT (1)..(1): Xaa can be Asp or Glu
  UNSURE (2)..(2): Xaa can be any naturally occurring amino acid
  UNSURE (4)..(4): Xaa can be any naturally occurring amino acid
  UNSURE (7)..(7): Xaa can be any naturally occurring amino acid
  UNSURE (9)..(15): Xaa can be any naturally occurring amino acid
  UNSURE (17)..(19): Xaa can be any naturally occurring amino acid
  VARIANT (20)..(20): Xaa can be Ala, Cys, Pro or Ser
  UNSURE (21)..(28): Xaa can be any naturally occurring amino acid
  VARIANT (29)..(29): Xaa can be Lys or Arg
  NON_CONS (29)..(30): The Xaa residue in position 29 and the Xaa residue in position 30 are separated by 28 to 32 intervening amino acid residues
  VARIANT (30)..(30): Xaa can be Lys or Arg
  NON_CONS (30)..(31): The Xaa residue in position 30 and the Xaa residue in position 31 are separated by 28 to 30 intervening amino acid residues
  VARIANT (31)..(31): Xaa can be Asp or Glu
  NON_CONS (31)..(32): The Xaa residue in position 31 and the Lys residue in position 32 are separated by 14 to 16 intervening amino acid residues
  UNSURE (33)..(34): Xaa can be any naturally occurring amino acid
XXGXNHXGXXXXXXXMXXXXXXXXXXXXXXXKXXS

SEQ ID NO: 20 - protein, 35 aa, Artificial sequence; identical to SEQ ID NO: 19
  narrow NANS domain
  VARIANT (1)..(1): Xaa can be Asp or Glu
  UNSURE (2)..(2): Xaa can be any naturally occurring amino acid
  UNSURE (4)..(4): Xaa can be any naturally occurring amino acid
  UNSURE (7)..(7): Xaa can be any naturally occurring amino acid
  UNSURE (9)..(15): Xaa can be any naturally occurring amino acid
  UNSURE (17)..(18): Xaa can be any naturally occurring amino acid
  UNSURE (19)..(19): Xaa can be any naturally occurring amino acid except Ile, Leu, and Met
  VARIANT (20)..(20): Xaa can be Ala or Ser
  UNSURE (21)..(28): Xaa can be any naturally occurring amino acid
  VARIANT (29)..(29): Xaa can be Lys or Arg
  NON_CONS (29)..(30): The Xaa residue in position 29 and the Xaa residue in position 30 are separated by 28 to 32 intervening amino acid residues
  VARIANT (30)..(30): Xaa can be Lys or Arg
  NON_CONS (30)..(31): The Xaa residue in position 30 and the Xaa residue in position 31 are separated by 28 to 30 intervening amino acid residues
  VARIANT (31)..(31): Xaa can be Asp or Glu
  NON_CONS (31)..(32): The Xaa residue in position 31 and the Lys residue in position 32 are separated by 14 to 16 intervening amino acid residues
  UNSURE (33)..(34): Xaa can be any naturally occurring amino acid

SEQ ID NO: 21 - protein, 348 aa, Syntrophorhabdus sp. PtaU1.Bin058
MESFSFGKSTVSKNGPCYIIAEIGNNHQGDLKIALKMIKTAAGMGVNAVKFQKRDNRTLYTKSMYNKPYENENSYGSTYGEHRDYLEFCWDKYVEMKKCAEDNDVEFMCTAFDFKSVDFLEQLGITSYKVASGDVTNTPLLEYIAQTKKPMFVSTGGATLEEIRVAYDTVTKYNDRVCLLHCVAGYPTEYEYLNLNTINILKKEFPDTLIGYSGHDYGILAPVVAYMLGAVVVEKHFTLNRAWKGTDHKFSLEPTGLYKMTRDLRRVDVSIGNGKKVVHAFEKDPISKMSKSLYASRKLQAGRVLTADDISIKSPGGGIPPYKMEEVLGKMLKINLPEEALFSFDYFE

SEQ ID NO: 22 - protein, 291 aa, Chloroflexi bacterium ADurb.Bin344
MGKTVKLGNRLVGEGQPVYVIAEIGINHNGDIGIAKRMIRSAYAAGVDAVKFQKRTPAVCVPLSEQSKMRETPWGYMSYLEYREHVEFWEDEYHEIDELCHELGIDWFASVWDVESIDFLMKFDPIAYKVPSASLTDHELLDKLLSTRKPIILSTGMSTMDQIRQSVSLFGNLDNLIITHATSAYPCKPEELNLRMIPKLIEEFDCPIGYSGHEVGLIPSVISVALGACLVERHFTLDRAMWGTDQAASVEPQGMEKLVKYIRVTEASLGDGIKKVYDDELPSLKKLRRVQ

SEQ ID NO: 23 - protein, 296 aa, Ignavibacteria bacterium UBA2353
MRKSVLIGDKAVGDKHSVYVIAEIGINHNGSVELAKKMIDSAAIAGCDAVKFQKRTPELCVPREQWDIERDTPWGRMTYLEYRHKVEFGFNEFDEIDQYCKQKNIQWFASCWDEPSVEFIEQFNPPCYKAASASLTDIPLLQTMQNTGKPLIISTGMSTMEEITEAIDSLDTSALLIAHSTSSYPCKIEELNLNMIRTLRSLYPDIPIGYSGHEVGLAPTWSAVTLGACFVERHITLDRAMWGTDQAASVEMVGLIRLVSNIRDIEKALGDGIKRVYDSELSSRKKLRRVSTELVA

SEQ ID NO: 24 - protein, 305 aa, Ignavibacteria bacterium UBA7675
MTRNIVSIGSRKIGDGKPVFIIAEIGINHNGSVDVAKKMIQSAAAAGCDAVKFQKRTPELCVPKDQWNIERDTPWGRMTYIDYRHKVEFSRVQYEELDYYCREQGILWMASCWDEEAVDFIENFDTPLYKAASASLTDIPLLKKMKATGKPLMISTGMSTMEDIRAAVDALGEDNTLIAHSTSTYPCKLEELNLRVIHTLKDEYRAIPIGYSGHETGLAPTLAAVAMGACFVERHITLDRAMWGTDQAASVEVGGLFRLVSNIRDIEKSMGDGVKRVFDSEISGMKKLRRVPGAQPVQEERTVLA

SEQ ID NO: 25 - protein, 348 aa, Nitrospirae bacterium strain NC_groundwater_1772_Pr3_B-0.1um_56_14
MKTITFGRSRVTEGGPCYIIAEIGNNHQGDLKTALKMIKTAAGMGVDAVKFQKRDNKSMFTKAMYYKPYDNENSYGETYGEHREFLEFGWDEYVELKKCADENDVEFMCTAFDFKSADFLEKLGIGSYKIASGDLTNTPLLEYIAKFGKPMFVSTGAASMDEIRMAYEAVLKHNNQLCLLHCTAAYPAEYEDLNLKFIDTMRKEFPAALIGFSGHDYGILAPVVAYMLGAVVVEKHFTLNRAWKGTDHRFSLEPTGLYKVIRDLRRVDKALGDGTKIVKKFERDARNKMGKSLYAARNLQAGTVITADDIAIKSPDGGMPPYMFNDIVGKTMKADLAEDKQFAPEVLA

SEQ ID NO: 26 - protein, 292 aa, Desulfobacterales bacterium strain NC_groundwater_1491_Pr4_B-0.1um_43_5
MDKVQKNLKLGNRSIIGPGNPVYIIAEIGINHNGSIDLAKEMIRSAHKVGVNAVKFQKRNPEECVPLEQRDRMRETPWGYISYMDYRYKVEFGEKEYRDIDNLCKNLGIEWFVSCWDKTSVDFMERFDPICYKIPSAHITNKELLLYLKKTRRPLILSTGMSTMSEIRQAVELVGTDTLALAHCTSSYPAKLEELNLRMILTLRKMYDCPIGYSGHEVGLATSVVAVSLGACFVERHFTLDRASWGSDQASSVEIAGLARLVKDIRSVEIALGDGEKVVYESEMEIRERLRA

SEQ ID NO: 27 - protein, 305 aa, uncultured delta proteobacterium strain AM-lipid-02-D9_bin-1227
MSGKPFWEDFIQTEPWPIVRKPYIIAEIGINHNGDIILAKQMIKSAYQCGCQAVKFQKRTPELCVPKHQRDLLRETPWGLITYMEYRKKIEFGREEYDEIDSFCRELGVDWFASAWDIPSLEFLRKYNSPYNKIASAMLTHRELVRQVAQEGKMTFISTGMSEYADIDYIVKLFRDIGCPFILMHSVSEYPLINYKVNLRQITALRTRYNCPVGNSGHESTMLPSLLAIMMGAVAIERHFTIDRAYWGTDQAASLETRGMATLMNYINQIPYVLGTGERVITEVERANAKKLRFFIEEPERNQSK

SEQ ID NO: 28 - protein, 359 aa, Desulfobaculum xiamenense
MRTVPFIRLPHGAAIGHEAPCFVIAEIGNNHQGDLDIARAMVRSAAEAGADAVKFQKRDNAALFTREGLDAPYTGPNSFGPTYGEHRRALELDFEQLEQLKRQAEALGMVFFASAWDMPSLDGLAAMGVELFKVASADLVNVPMLRRIAALGRPVLLSTGMSTLAEIDLAVNELDRSRTVLLHCNSSYPCPPEETAIPVMNRLRTRYGLPVGYSGHEGGIAPSLAAVAHGACVVERHFTLDRTMRGTDHTASLDPAEFAHLVLMIRETEAALRITDKRVFEAEARCAAKLRKSIVAARNLTAGTMLTESDITVRCPGTGISPAAWDEVIGRVLVRDIAADGQLAWDAMLAKQPLRAAAE

SEQ ID NO: 29 - protein, 73 aa, Artificial sequence
  domain 1 phosphatases
  UNSURE (2)..(2): Xaa can be any naturally occurring amino acid
  UNSURE (5)..(6): Xaa can be any naturally occurring amino acid
  UNSURE (9)..(16): Xaa can be any naturally occurring amino acid
  UNSURE (18)..(26): Xaa can be any naturally occurring amino acid
  UNSURE (28)..(42): Xaa can be any naturally occurring amino acid
  VARIANT (43)..(43): Xaa can be Ile, Leu or Val
  UNSURE (44)..(44): Xaa can be any naturally occurring amino acid
  VARIANT (45)..(45): Xaa can be Ser or Thr
  UNSURE (46)..(54): Xaa can be any naturally occurring amino acid
  UNSURE (56)..(58): Xaa can be any naturally occurring amino acid
  NON_CONS (59)..(60): The Leu residue in position 59 and the Lys residue in position 60 are separated by 10 to 11 intervening amino acid residues
  NON_CONS (60)..(61): The Lys residue in position 60 and the Gly residue in position 61 are separated by 21 intervening amino acid residues
  UNSURE (62)..(62): Xaa can be any naturally occurring amino acid
  UNSURE (64)..(65): Xaa can be any naturally occurring amino acid
  NON_CONS (66)..(67): The Asp residue in position 66 and the Gly residue in position 67 are separated by 32 intervening amino acid residues
  UNSURE (68)..(68): Xaa can be any naturally occurring amino acid
  UNSURE (70)..(71): Xaa can be any naturally occurring amino acid
  VARIANT (73)..(73): Xaa can be Asp or Glu
DXDGXXTDXXXXXXXXGXXXXXXXXXDXXXXXXXXXXXXXXXXXXXXXXXXXXXRXXXLKGXDXXDGXGXXRX

SEQ ID NO: 30 - protein, 241 aa, Candidatus Magnetomorum sp. HK-1
MPSLKAIFFDLDNTLVDCSTADIRTYEILASIARDKVRGINSPALINHFQKQLINVPFDPEKQIPVHPWRTILWTKALKSENIDDIDLAEKLNIAFHRERLAFYTFIYGSQKMLKKLLKLYTCVIITNGDSEIQRPKLKACKAQKYFSHIIVGGEEPYEKPHPSIFKKACEMAKCKPESAIIIGDSLETDIKGGLDAGLASTIWINPKNKPIPEKGPVPHYQAVSVLELPLIIQHIQQQQQ

SEQ ID NO: 31 - protein, 35 aa, Artificial sequence
  domain 2 phosphatases
  UNSURE (2)..(2): Xaa can be any naturally occurring amino acid
  UNSURE (4)..(4): Xaa can be any naturally occurring amino acid
  VARIANT (6)..(6): Xaa can be Ile or Leu
  NON_CONS (6)..(7): The Xaa residue in position 6 and the Thr residue in position 7 are separated by 110 to 160 intervening amino acid residues
  UNSURE (10)..(13): Xaa can be any naturally occurring amino acid
  UNSURE (15)..(16): Xaa can be any naturally occurring amino acid
  VARIANT (18)..(18): Xaa can be Ile or Leu
  NON_CONS (18)..(19): The Xaa residue in position 18 and the Xaa residue in position 19 are separated by 20 to 23 intervening amino acid residues
  VARIANT (19)..(19): Xaa can be Lys or Arg
  UNSURE (21)..(23): Xaa can be any naturally occurring amino acid
  VARIANT (24)..(24): Xaa can be Ile or Leu
  VARIANT (25)..(25): Xaa can be Phe, Trp or Tyr
  NON_CONS (25)..(26): The Xaa residue in position 25 and the Gly residue in position 26 are separated by 17 intervening amino acid residues
  VARIANT (27)..(27): Xaa can be Asp or Asn
  UNSURE (28)..(31): Xaa can be any naturally occurring amino acid
  VARIANT (33)..(33): Xaa can be Ile, Leu or Val
  UNSURE (34)..(34): Xaa can be any naturally occurring amino acid
DXDXTXTNGXXXXQXXKXXPXXXXXGXXXXXDXXG

SEQ ID NO: 32 - protein, 164 aa, Bacteroides thetaiotaomicron
MKEIKLILTDIDGVWTDGGMFYDQTGNEWKKFNTSDSAGIFWAHNKGIPVGILTGEKTEIVRRRAEKLKVDYLFQGVVDKLSAAEELCNELGINLEQVAYIGDDLNDAKLLKRVGIAGVPASAPFYIRRLSTIFLEKRGGEGVFREFVEKVLGINLEDFIAVIQ

SEQ ID NO: 33 - protein, 722 aa, Mus musculus (strain C57BL/6J)
MEKNGNNRKLRVCVATCNRADYSKLAPIMFGIKTEPAFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKCKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERISLILQMCVEAAAEAVKLNCRILGVGISTGGRVNPQEGVVLHSTKLIQEWNSVDLRTPLSDTLHLPVWVDNDGNCAAMAERKFGQGKGQENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNVKAQSILRTAGTALGLGVVNILHTMNPSLVILSGVLASHYIHIVKDVIRQQALSSVQDVDVVVSDLVDPALLGAASMVLDYTTRRIH

SEQ ID NO: 34 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 35 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 36 - protein, 609 aa, Escherichia coli K-12 MG1655
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 37 - protein, 445 aa, Escherichia coli K-12 MG1655
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 38 - protein, 456 aa, Escherichia coli K-12 MG1655
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 39 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 40 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 41 - protein, 308 aa, Pasteurella multocida subsp. multocida str. Pm70
MNLIICCTPLQVLIAEKIIAKFPHMPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE

SEQ ID NO: 42 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 43 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 44 - protein, 498 aa, Photobacterium sp. JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 45 - protein, 380 aa, Mus musculus
MRNCKMARVASVLGLVMLSVALLILSLISYVSLKKENIFTTPKYASPGAPRMYMFHAGFRSQFALKFLDQSFVPITNSLTHELQEKPSKWTFNRTAFLHQRQEILQHVDVIKNFSLTKSSVRIGQLMHYDYSSHKYVFSISNNFRSLLPDVSPIMNKRYNVCAVVGNSGILTGSQCGQEIDKSDFVFRCNFAPTEAFHKDVGRKTNLTTFNPSILEKYYNNLLTIQDRNNFFLSLKKLDGAILWIPAFFFHTSATVTRTLVDFFVEHRGQLKVQLAWPGNIMQHVNRYWKNKHLSPKRLSTGILMYTLASAICEEIHLYGFWPFGFDPNTREDLPYHYYDKKGTKFTTKWQESHQLPAEFQLLYRMHGEGLTKLTLSHCA

SEQ ID NO: 46 - protein, 417 aa, Escherichia coli K-12 MG1655 [sequence omitted]

SEQ ID NO: 47 - protein, 415 aa, Escherichia coli W
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 48 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 49 - protein, 504 aa, Bifidobacterium adolescentis [sequence omitted]

SEQ ID NO: 50 - protein, 299 aa, Helicobacter Pylori UA1234
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 51 - protein, 478 aa, Helicobacter Pylori UA1234
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 52 - protein, 264 aa, Escherichia coli K-12 MG1655
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 53 - protein, 391 aa, Escherichia coli K-12 MG1655
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 54 - protein, 456 aa, Escherichia coli K-12 MG1655
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 55 - protein, 478 aa, Escherichia coli K-12 MG1655
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 56 - protein, 373 aa, Escherichia coli K-12 MG1655 [sequence omitted]

SEQ ID NO: 57 - protein, 321 aa, Escherichia coli K-12 MG1655
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 58 - protein, 438 aa, Escherichia coli K-12 MG1655
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 59 - protein, 949 aa, Bacteroides fragilis NCTC 9343
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 60 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 61 - protein, 265 aa, Escherichia coli O55:H7
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 62 - protein, 275 aa, Neisseria meningitidis MC58
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 63 - protein, 338 aa, Escherichia coli K-12 MG1655
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 64 - protein, 221 aa, Campylobacter jejuni
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 65 - protein, 224 aa, Haemophilus influenzae
MTRIAIIPARAGSKGIKDKNLQLVGGVSLVGRAILAAQESGMFDQIVVTSDGENILKEATKYGAKAVARPESLAQSDTRTIDAILHCLETLNISQGTAALLQPTSPLRNALDIRNAMEIFLGGKYKSVVSACECEHHPYKSFTLEGTEVQPIHELTDFEAPRQKLPKSYRANGAIYTNDIQSLFEEKRFFIAPMRFYLMPTYRSIDIDSTLDLQLAESLISKEF

SEQ ID NO: 66 - protein, 496 aa, Escherichia coli K-12 MG1655
MSTTTQNIPWYRHLNRAQWRAFSAAWLGYLLDGFDFVLIALVLTEVQGEFGLTTVQAASLISAAFISRWFGGLMLGAMGDRYGRRLAMVTSIVLFSAGTLACGFAPGYITMFIARLVIGMGMAGEYGSSATYVIESWPKHLRNKASGFLISGFSVGAVVAAQVYSLVVPVWGWRALFFIGILPIIFALWLRKNIPEAEDWKEKHAGKAPVRTMVDILYRGEHRIANIVMTLAAATALWFCFAGNLQNAAIVAVLGLLCAAIFISFMVQSAGKRWPTGVMLMVVVLFAFLYSWPIQALLPTYLKTDLAYNPHTVANVLFFSGFGAAVGCCVGGFLGDWLGTRKAYVCSLLASQLLIIPVFAIGGANVWVLGLLLFFQQMLGQGIAGILPKLIGGYFDTDQRAAGLGFTYNVGALGGALAPIIGALIAQRLDLGTALASLSFSLTFVVILLIGLDMPSRVQRWLRPEALRTHDAIDGKPFSGAVPFGSAKNDLVKTKS

SEQ ID NO: 67 - protein, 352 aa, Aeromonas caviae
MDKIIKPFITINGRKIGPDYPPYIIAELSANHNGDINRAFAIMEEAKKAGADAIKLQTYTADTITFECDSEEFQIHGGLWDGKNLYQLYKEAQMPWEWHQPLFEKAKELGITIFSSPFDFTAVDLLEDLDAPAYKIASFELIDLPLIKRVAQTGKPMIMSTGMANAEEIAEAIATAQSNGCQELVVLHCVSGYPAPADQYNLRTIADMAERFGVLSGLSDHTIDNATAVAAVALGACLIEKHVTMDRNGGGADDSFSLEPHELAALCKDAKTAWSALGSVNYTRTEAEKGNVKFRRSLYVIRDIKAGDVLSNDNVRSIRPGFGLAPKYLEQVLGKTAIVDIKRGTPLSFEFI

SEQ ID NO: 68 - protein, 300 aa, Candidatus koribacter versatilis
MAANNGQQQHPAVRIGSKMVGKDQPCYVIAEIGINHNGDMDIAKRLISVAVGAGCDAVKFQKRTIDVVYTAAELAKPRENPFGATNGDLKRGLEFGLEEFKEIDRYCREVRMPWFASAWDEASVDFIAQFDVPCFKIASASLTDDNLLRHTRATGKPVMLSTGMSTVEQIDHAVDVLGKDNLILLQACSTYPAYYEELNLKAIHTLMDRYGVPVGYSGHETGLPASIAAAAMGACVLERHITLDRAMWGSDQAASLEPNGITQLVRYLRIVEKSMGDGVKRVLEREQPVIQKLRRVGGGQ

SEQ ID NO: 69 - protein, 338 aa, Legionella pneumophila
MTCFIIAEAGVNHNGDLQLAKELVYAAKESGADAVKFQTFKADTLVNKTVEKAEYQKNNAPESSTQYEMLKALELSEEDHYLLSELANSLGIEFMSTGFDEQSIDFLISLGVKRLKIPSGEITNVPYLQHCASKKLPLIISTGMCDLQEVRVAIDTVKPYYGNSLSDYLVLLHCTSNYPASYQDVNLKAMQTLADEFQLPVGYSDHTLGILVPTLAVGMGACVIEKHFTMDKSLPGPDHLASMDPEEMKNLVQSIRDAETVLGSGEKKPSDNELPIRALVRRSITLRRDLVKGAQISKEDLILLRPGTGIAPSEISNIVGSRLSMNLSAGTNFVMGTY

SEQ ID NO: 70 - protein, 337 aa, Methanocaldococcus jannaschii
MEKIKIGDRYVGKGEPTFIIAEGGLNHNGDIDIGKELVKEAKKCGADAIKFQSYHTEDFISKKSEYYELFKSLELSEEEFYELKEYAEKIGIMFISTPLDLKYVDILNKMNVPAFKIASGDLTFYPLLEKVAKTGKPVILSTGMSDIGEIWEAVKVLENNGCRDIILLHCISSYPTPYEDVNLNAIKTLKSIFNIPVGYSDHTLGILAPVVSVALGADVIEKHFTLDKNMEGPDHALSADPEEFKEMVNNIRLVEKMLGSGEKIPMPSERDVIVEARRSIVAKRNIKKGEYLSVDNISFKRPGRGIETKYLSIILNRKIKNDKEEDDIIYWDDLLGD

SEQ ID NO: 71 - protein, 350 aa, Moritella viscosa
MTEQYITIDGRKIGPNFSPYIIAELSANHNGDINRAFAIMEAAKKAGADAIKLQTYTQDTITMDCDSDEFQIKGGLWHGQSLYQLYTSAHMPWEWHQPLFAKAKELDITIFSSPFDFTAVDLLEELDAPAYKIASFEIVDLPLIKRVAQTGKPMIISTGMADQSEIEIAIQTAKDNGCDELVVLHCVSGYPAPAAQYNLRTIADIGQRFDVLAGLSDHTIDNATAVVSVAFGACVIEKHVTLDRNAGGADDSFSLEPDELARLCRDTYTAWQAMGNVNYERTPAEQGNVKFRRSLYAVKDIAAGELLTADNVRSIRPGFGLEPKYYDQVLGKKANVIISKGTALSFGLIS

SEQ ID NO: 72 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 73 - protein, 792 aa, Escherichia coli K-12 MG1655
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 74 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVKCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWKGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGRVGADETVVGHTAKAEDLDLDGLDTPIEDVKEALTAPAEQWANDVQDNAEYLTFLGPRVPAEVHSQFDALKARISAAHA

SEQ ID NO: 75 - protein, 540 aa, Escherichia coli K-12 MG1655
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 76 - protein, 213 aa, Escherichia coli K-12 MG1655
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 77 - protein, 565 aa, Escherichia coli K-12 MG1655
MEPKTKKQRSLYIPYAGPVLLEFPLLNKGSAFSMEERRNFNLLGLLPEVVETIEEQAERAWIQYQGFKTEIDKHIYLRNIQDTNETLFYRLVNNHLDEMMPVIYTPTVGAACERFSEIYRRSRGVFISYQNRHNMDDILQNVPNHNIKVIVVTDGERILGLGDQGIGGMGIPIGKLSLYTACGGISPAYTLPVVLDVGTNNQQLLNDPLYMGWRNPRITDDEYYEFVDEFIQAVKQRWPDVLLQFEDFAQKNAMPLLNRYRNEICSFNDDIQGTAAVTVGTLIAASRAAGGQLSEKKIVFLGAGSAGCGIAEMIISQTQREGLSEEAARQKVFMVDRFGLLTDKMPNLLPFQTKLVQKRENLSDWDTDSDVLSLLDVVRNVKPDILIGVSGQTGLFTEEIIREMHKHCPRPIVMPLSNPTSRVEATPQDIIAWTEGNALVATGSPFNPVVWKDKIYPIAQCNNAFIFPGIGLGVIASGASRITDEMLMSASETLAQYSPLVLNGEGMVLPELKDIQKVSRAIAFAVGKMAQQQGVAVKTSAEALQQAIDDNFWQAEYRDYRRTSI

SEQ ID NO: 78 - protein, 759 aa, Escherichia coli K-12 MG1655
MDDQLKQSALDFHEFPVPGKIQVSPTKPLATQRDLALAYSPGVAAPCLEIEKDPLKAYKYTARGNLVAVISNGTAVLGLGNIGALAGKPVMEGKGVLFKKFAGIDVFDIEVDELDPDKFIEVVAALEPTFGGINLEDIKAPECFYIEQKLRERMNIPVFHDDQHGTAIISTAAILNGLRVVEKNISDVRMVVSGAGAAAIACMNLLVALGLQKHNIVVCDSKGVIYQGREPNMAETKAAYAVVDDGKRTLDDVIEGADIFLGCSGPKVLTQEMVKKMARAPMILALANPEPEILPPLAKEVRPDAIICTGRSDYPNQVNNVLCFPFIFRGALDVGATAINEEMKLAAVRAIAELAHAEQSEVVASAYGDQDLSFGPEYIIPKPFDPRLIVKIAPAVAKAAMESGVATRPIADFDVYIDKLTEFVYKTNLFMKPIFSQARKAPKRVVLPEGEEARVLHATQELVTLGLAKPILIGRPNVIEMRIQKLGLQIKAGVDFEIVNNESDPRFKEYWTEYFQIMKRRGVTQEQAQRALISNPTVIGAIMVQRGEADAMICGTVGDYHEHFSVVKNVFGYRDGVHTAGAMNALLLPSGNTFIADTYVNDEPDAEELAEITLMAAETVRRFGIEPRVALLSHSNFGSSDCPSSSKMRQALELVRERAPELMIDGEMHGDAALVEAIRNDRMPDSSLKGSANILVMPNMEAARISYNLLRVSSSEGVTVGPVLMGVAKPVHVLTPIASVRRIVNMVALAVVEAQTQPL

SEQ ID NO: 79 - protein, 652 aa, Escherichia coli K-12 MG1655
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 80 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG